SEQUENCE LISTING (158 sequences)

SEQ ID NO: 1 - protein, 450 aa, Homo sapiens
  MISC_FEATURE: HUMAN CHRDL-1 PROTEIN
MGGMKYIFSLLFFLLLEGGKTEQVKHSETYCMFQDKKYRVGERWHPYLEPYGLVYCVNCICSENGNVLCSRVRCPNVHCLSPVHIPHLCCPRCPDSLPPVNNKVTSKSCEYNGTTYQHGELFVAEGLFQNRQPNQCTQCSCSEGNVYCGLKTCPKLTCAFPVSVPDSCCRVCRGDGELSWEHSDGDIFRQPANREARHSYHRSHYDPPPSRQAGGLSRFPGARSHRGALMDSQQASGTIVQIVINNKHKHGQVCVSNGKTYSHGESWHPNLRAFGIVECVLCTCNVTKQECKKIHCPNRYPCKYPQKIDGKCCKVCPGKKAKELPGQSFDNKGYFCGEETMPVYESVFMEDGETTRKIALETERPPQVEVHVWTIRKGILQHFHIEKISKRMFEELPHFKLVTRTTLSQWKIFTEGEAQISQMCSSRVCRTELEDLVKVLYLERSEKGHC

SEQ ID NO: 2 - DNA, 1353 bp, Homo sapiens
  misc_feature: HUMAN CHRDL-1 OPEN READING FRAME cDNA
atgggaggcatgaaatacatcttttcgttgttgttctttcttttgctagaaggaggcaaaacagagcaagtaaaacattcagagacatattgcatgtttcaagacaagaagtacagagtgggtgagagatggcatccttacctggaaccttatgggttggtttactgcgtgaactgcatctgctcagagaatgggaatgtgctttgcagccgagtcagatgtccaaatgttcattgcctttctcctgtgcatattcctcatctgtgctgccctcgctgcccagactccttacccccagtgaacaataaggtgaccagcaagtcttgcgagtacaatgggacaacttaccaacatggagagctgttcgtagctgaagggctctttcagaatcggcaacccaatcaatgcacccagtgcagctgttcggagggaaacgtgtattgtggtctcaagacttgccccaaattaacctgtgccttcccagtctctgttccagattcctgctgccgggtatgcagaggagatggagaactgtcatgggaacattctgatggtgatatcttccggcaacctgccaacagagaagcaagacattcttaccaccgctctcactatgatcctccaccaagccgacaggctggaggtctgtcccgctttcctggggccagaagtcaccggggagctcttatggattcccagcaagcatcaggaaccattgtgcaaattgtcatcaataacaaacacaagcatggacaagtgtgtgtttccaatggaaagacctattctcatggcgagtcctggcacccaaacctccgggcatttggcattgtggagtgtgtgctatgtacttgtaatgtcaccaagcaagagtgtaagaaaatccactgccccaatcgatacccctgcaagtatcctcaaaaaatagacggaaaatgctgcaaggtgtgtccaggtaaaaaagcaaaagaacttccaggccaaagctttgacaataaaggctacttctgcggggaagaaacgatgcctgtgtatgagtctgtattcatggaggatggggagacaaccagaaaaatagcactggagactgagagaccacctcaggtagaggtccacgtttggactattcgaaagggcattctccagcacttccatattgagaagatctccaagaggatgtttgaggagcttcctcacttcaagctggtgaccagaacaaccctgagccagtggaagatcttcaccgaaggagaagctcagatcagccagatgtgttcaagtcgtgtatgcagaacagagcttgaagatttagtcaaggttttgtacctggagagatctgaaaagggccactgttag

SEQ ID NO: 3 - protein, 425 aa, Mus musculus
  MISC_FEATURE: MOUSE CHRDL-1 PROTEIN SEQUENCE
EQVKHSDTYCVFQDKKYRVGEKWHPYLEPYGLVYCVNCICSENGNVLCSRVRCPSLHCLSPVHIPHLCCPRCPDSLPPVNNKVTSKSCEYNGTTYQHGELFIAEGLFQNRQPNQCSQCSCSEGNVYCGLKTCPKLTCAFPVSVPDSCCRVCRGDAELSWEHADGDIFRQPANREARHSYLRSPYDPPPNRQAGGLPRFPGSRSHRGAVIDSQQASGTIVQIVINNKHKHGQVCVSNGKTYSHGESWHPNLRAFGIVECVLCTCNVTKQECKKIHCPNRYPCKYPQKIDGKCCKVCPEEPPSQNFDSKGSFCGEETMPVYESVFMEDGETTRKVALETERPPQVEVHVWTIQKGILQHFHIEKISKRMFGELHHFKLVTRTTLNQWKLFTEGEAQLSQMCSSQVCRTELEDLVQVLYLGRPEKDHC

SEQ ID NO: 4 - DNA, 1275 bp, Mus musculus
  misc_feature: MOUSE CHRDL-1 cDNA SEQUENCE
gaacaagtaaaacactcagacacatattgcgtgtttcaagacaagaagtatagagtgggtgagaaatggcatccctacctggaaccgtatggactggtttactgtgtgaactgcatctgctctgagaatgggaatgtgctttgcagccgagtcagatgtccaagtcttcattgcctttcacccgtgcatattcctcatctctgttgcccccgctgcccagactccttaccaccagtgaacaataaggtgaccagcaagtcatgcgaatacaatggaaccacttaccaacatggagaactgttcatagctgaagggctctttcagaaccggcaacccaatcagtgcagtcagtgtagctgctcggaggggaatgtatactgtggtctcaagacttgccccaaactgacctgtgcattcccagtctctgttccagattcttgctgccgagtatgcagaggggatgcagaattatcgtgggaacatgcggatggtgatatcttccggcaacctgccaacagagaagcaagacattcttacctccgttccccctacgatcctccaccaaacagacaagctggaggtcttccccgctttcctgggagcagaagtcaccggggagctgttatagattcccagcaagcatccgggaccatcgtgcagattgtcatcaataacaagcacaaacatggacaagtgtgtgtttccaatggaaagacctactctcatggagagtcctggcacccaaatctacgagcatttggcattgtggaatgtgtactatgcacttgtaatgtcaccaagcaagaatgtaagaaaatccactgccccaatcgatacccctgcaagtatcctcaaaaaatagatggaaagtgctgcaaggtgtgcccagaagaacctccaagccaaaactttgacagcaaaggttccttttgtggagaagaaaccatgcctgtatatgagtctgtgttcatggaggatggagagacaaccagaaaagtagcactggagaccgagagaccacctcaagtagaggtccacgtttggactattcaaaagggcattctccagcacttccacattgagaagatttccaagaggatgtttggggagctccatcatttcaagctagttactcggaccaccttgaaccagtggaagctcttcactgaaggagaagctcagctcagccagatgtgctcaagtcaggtgtgcagaacagagctggaagatttagtccaggttttgtacctggggagacctgaaaaggaccactgt

SEQ ID NO: 5 - DNA, 33 bp, Mus musculus
  misc_feature: TC1E3.1 ANTIBODY CDR1
aaggccagtcaggatgtgagtactgctgtagcc

SEQ ID NO: 6 - DNA, 21 bp, Mus musculus
  misc_feature: TC1E3.1 ANTIBODY CDR2
tgggcatccacccggcacact

SEQ ID NO: 7 - DNA, 27 bp, ARTIFICIAL SEQUENCE
  HYBRID SEQUENCE
cagcaacattatagcactccgtggacg

SEQ ID NO: 8 - protein, 11 aa, Mus musculus
  MISC_FEATURE: TC1E3.1 ANTIBODY CDR1
KASQDVSTAVA

SEQ ID NO: 9 - protein, 7 aa, Mus musculus
  MISC_FEATURE: TC1E3.1 ANTIBODY CDR2
WASTRHT

SEQ ID NO: 10 - protein, 9 aa, Mus musculus
  MISC_FEATURE: TC1E3.1 ANTIBODY CDR3
QQHYSTPWT

SEQ ID NO: 11 - DNA, 33 bp, Mus musculus; identical to SEQ ID NO: 5
  misc_feature: TC2E1.1 ANTIBODY CDR1

SEQ ID NO: 12 - DNA, 21 bp, Mus musculus
  misc_feature: TC2E1.1 ANTIBODY CDR1
tgggcatccacccggcacact

SEQ ID NO: 13 - DNA, 27 bp, Mus musculus
  misc_feature: TC2E1.1 ANTIBODY CDR3
cagcaacattatagcactccgtggacg

SEQ ID NO: 14 - protein, 11 aa, Mus musculus
  MISC_FEATURE: TC2E1.1 ANTIBODY CDR1
KASQDVSTAVA

SEQ ID NO: 15 - protein, 7 aa, Mus musculus
  MISC_FEATURE: TC2E1.1 ANTIBODY CDR2
WASTRHT

SEQ ID NO: 16 - protein, 9 aa, Mus musculus
  MISC_FEATURE: TC2E1.1 ANTIBODY CDR3
QQHYSTPWT

SEQ ID NO: 17 - DNA, 15 bp, Mus musculus
  misc_feature: TC1E3.1 CDRS ANTIBODY CDR1
gagtatactatacac

SEQ ID NO: 18 - DNA, 51 bp, Mus musculus
  misc_feature: TC1E3.1 ANTIBODY CDR2
tggttttaccctggaagtggtagtataaagtacaatgagaaattcagggac

SEQ ID NO: 19 - DNA, 27 bp, Mus musculus
  misc_feature: TC1E3.1 ANTIBODY CDR3
ctcgaccgggactatgctatggactac

SEQ ID NO: 20 - protein, 5 aa, Mus musculus
  MISC_FEATURE: TC1E3.1 ANTIBODY CDR1
EYTIH

SEQ ID NO: 21 - protein, 17 aa, Mus musculus
  MISC_FEATURE: TC1E3.1 ANTIBODY CDR2
WFYPGSGSIKYNEKFRD

SEQ ID NO: 22 - protein, 9 aa, Mus musculus
  MISC_FEATURE: TC1E3.1 ANTIBODY CDR3
LDRDYAMDY

SEQ ID NO: 23 - DNA, 15 bp, Mus musculus
  misc_feature: TC2E1.1 ANTIBODY CDR1
gagtatactataaac

SEQ ID NO: 24 - DNA, 51 bp, Mus musculus; identical to SEQ ID NO: 18
  misc_feature: TC2E1.1 ANTIBODY CDR2

SEQ ID NO: 25 - DNA, 27 bp, Mus musculus
  misc_feature: TC2E1.1 ANTIBODY CDR3
ctcgaccgggactatgctatggactac

SEQ ID NO: 26 - protein, 5 aa, Mus musculus
  MISC_FEATURE: TC2E1.1 ANTIBODY CDR1
EYTIN

SEQ ID NO: 27 - protein, 17 aa, Mus musculus
  MISC_FEATURE: TC2E1.1 ANTIBODY CDR2
WFYPGSGSIKYNEKFRD

SEQ ID NO: 28 - protein, 9 aa, Mus musculus
  MISC_FEATURE: TC2E1.1 ANTIBODY CDR3
LDRDYAMDY

SEQ ID NO: 29 - protein, 108 aa, Mus musculus
  MISC_FEATURE: TC1E3.1 ANTIBODY LIGHT CHAIN VARIABLE DOMAIN
DIVMTQSHKFMSTSVGDRVSITCKASQDVSTAVAWYQQKPGQSPKLLIYWASTRHTGVPDRFTGSGSGTDYTLTISSVQAEDLALYYCQQHYSTPWTFGGGTKLEIKR

SEQ ID NO: 30 - DNA, 324 bp, Mus musculus
  misc_feature: TC1E3.1 ANTIBODY LIGHT CHAIN VARIABLE DOMAIN
gacattgtgatgacccagtctcacaaattcatgtccacatcagtaggagacagggtcagcatcacctgcaaggccagtcaggatgtgagtactgctgtagcctggtatcaacaaaaaccagggcaatctcctaaactactgatttactgggcatccacccggcacactggagtccctgatcgcttcacaggcagtggatctgggacagattatactctcaccatcagcagtgtgcaggctgaagacctggcactttattactgtcagcaacattatagcactccgtggacgttcggtggaggcaccaagctggaaatcaaacgg

SEQ ID NO: 31 - protein, 118 aa, Mus musculus
  MISC_FEATURE: TC1E3.1 ANTIBODY HEAVY CHAIN VARIABLE DOMAIN
QVQLQQSGAELVKPGASVKLSCKASGYTFTEYTIHWVKQRSGQGLEWIGWFYPGSGSIKYNEKFRDKATLTADKSSSTVYMELSRLTSEDSAVYFCARLDRDYAMDYWGQGTSVTVSS

SEQ ID NO: 32 - DNA, 354 bp, Mus musculus
  misc_feature: TC1E3.1 ANTIBODY HEAVY CHAIN VARIABLE DOMAIN
caggtccagctgcagcagtctggagctgagctggtgaaacccggggcatcagtgaagctgtcctgcaaggcttctggctacaccttcactgagtatactatacactgggtaaagcagaggtctggacagggtcttgagtggattgggtggttttaccctggaagtggtagtataaagtacaatgagaaattcagggacaaggccacattgactgcggacaaatcctccagcacagtctatatggagcttagtagattgacatctgaagactctgcggtctatttctgtgcaagactcgaccgggactatgctatggactactggggtcaaggaacctcagtcaccgtctcctca

SEQ ID NO: 33 - protein, 108 aa, Mus musculus; identical to SEQ ID NO: 29
  MISC_FEATURE: TC2E1.1 ANTIBODY LIGHT CHAIN VARIABLE DOMAIN

SEQ ID NO: 34 - protein, 324 aa, Mus musculus
  MISC_FEATURE: TC2E1.1 ANTIBODY LIGHT CHAIN VARIABLE DOMAIN
GACATTGTGATGACCCAGTCTCACAAATTCATGTCCACATCAGTAGGAGACAGGGTCAGCATCACCTGCAAGGCCAGTCAGGATGTGAGTACTGCTGTAGCCTGGTATCAACAAAAACCAGGGCAATCTCCTAAACTACTGATTTACTGGGCATCCACCCGGCACACTGGAGTCCCTGATCGCTTCACAGGCAGTGGATCTGGGACAGATTATACTCTCACCATCAGCAGTGTGCAGGCTGAAGACCTGGCACTTTATTACTGTCAGCAACATTATAGCACTCCGTGGACGTTCGGTGGAGGCACCAAGCTGGAAATCAAACGG

SEQ ID NO: 35 - protein, 118 aa, Mus musculus
  MISC_FEATURE: TC2E1.1 ANTIBODY HEAVY CHAIN VARIABLE DOMAIN
QVQLQQSGAELVKPGASVKLSCKASGYTFTEYTINWVKQRSGQGLEWIGWFYPGSGSIKYNEKFRDKATLTADKSSSTVYMELSRLTSEDSAVYFCARLDRDYAMDYWGQGTSVTVSS

SEQ ID NO: 36 - DNA, 354 bp, Mus musculus
  misc_feature: TC2E1.1 ANTIBODY HEAVY CHAIN VARIABLE DOMAIN
caggtccagctgcagcagtctggagctgagctggtgaaacccggggcatcagtgaagctgtcctgcaaggcttctggctacaccttcactgagtatactataaactgggtaaagcagaggtctggacagggtcttgagtggattgggtggttttaccctggaagtggtagtataaagtacaatgagaaattcagggacaaggccacattgactgcggacaaatcctccagcacagtctatatggagcttagtagattgacatctgaagactctgcggtctatttctgtgcaagactcgaccgggactatgctatggactactggggtcaaggaacctcagtcaccgtctcctca

SEQ ID NO: 37 - protein, 11 aa, Mus musculus
  MISC_FEATURE: 2G2.2 ANTIBODY LIGHT CHAIN VARIABLE REGION, CDR-L1
RASENIYYSLA

SEQ ID NO: 38 - protein, 7 aa, Mus musculus
  MISC_FEATURE: 2G2.2 ANTIBODY LIGHT CHAIN VARIABLE REGION, CDR-L2
NANSLED

SEQ ID NO: 39 - protein, 9 aa, Mus musculus
  MISC_FEATURE: 2G2.2 ANTIBODY LIGHT CHAIN VARIABLE REGION, CDR-L3
KQAYDVPLT

SEQ ID NO: 40 - protein, 5 aa, Mus musculus
  MISC_FEATURE: 2G2.2 ANTIBODY HEAVY CHAIN VARIABLE REGION, CDR-H1
SYWMH

SEQ ID NO: 41 - protein, 17 aa, Mus musculus
  MISC_FEATURE: 2G2.2 ANTIBODY HEAVY CHAIN VARIABLE REGION, CDR-H2
NINPSNGGTNYNEKFKS

SEQ ID NO: 42 - protein, 12 aa, Mus musculus
  MISC_FEATURE: 2G2.2 ANTIBODY HEAVY CHAIN VARIABLE REGION, CDR-H3
SKNNYVAYYFDY

SEQ ID NO: 43 - protein, 108 aa, Mus musculus
  MISC_FEATURE: 2G2.2 ANTIBODY LIGHT CHAIN VARIABLE DOMAIN
DIQMTQSPASLAASVGETVTITCRASENIYYSLAWYQQKQGKSPQLLIYNANSLEDGVPSRFSGSGSGTQYSMKINSMQPEDTATYFCKQAYDVPLTFGAGTKLELKR

SEQ ID NO: 44 - DNA, 324 bp, Mus musculus
  misc_feature: 2G2.2 ANTIBODY LIGHT CHAIN VARIABLE DOMAIN
gacatccagatgactcagtctccagcctccctggctgcatctgtgggagaaactgtcaccatcacatgtcgagcaagtgagaacatttactacagtttagcttggtatcagcagaagcaagggaaatctcctcagctcctgatctataatgcaaacagcttggaagatggtgtcccatcgaggttcagtggcagtggatctgggacacagtattctatgaagatcaacagcatgcagcctgaagataccgcaacttatttctgtaaacaggcttatgacgttcctctcacgttcggtgctgggaccaagctggagctgaaacgg

SEQ ID NO: 45 - protein, 120 aa, Mus musculus
QVQLQQRGTELVKPGASVKLSCKASGYTFTSYWMHWVKQRPGQGLERIGNINPSNGGTNYNEKFKSKATLTVDKSSSTAYMQLSSLTSEDSAVYYCARSKNNYVAYYFDYWGQGTTLTVS

SEQ ID NO: 46 - DNA, 363 bp, Homo sapiens
  misc_feature: 2G2.2 ANTIBODY HEAVY CHAIN VARIABLE DOMAIN
caggtccaactgcagcagcgtgggactgaactggtgaagcctggggcttcagtgaagctgtcctgcaaggcttctggctacaccttcaccagctactggatgcactgggtgaagcagaggcctggacaaggccttgagcggattggaaatattaatcctagcaatggtggtactaactacaatgagaagttcaagagtaaggccacactgactgtagacaaatcctccagcacagcctacatgcagctcagcagcctgacatctgaggactctgcggtctattattgtgcaagatcgaagaataactacgtggcgtactactttgactactggggccaaggcaccactctcacagtctcctca

SEQ ID NO: 47 - protein, 108 aa, Artificial Sequence
  huz-LC1E3.1_VK HYBRID SEQUENCE
DIQMTQSPSSLSASVGDRVTITCKASQDVSTAVAWYQQKPGKVPKLLIYWASTRHTGVPSRFSGSGSGTDFTLTISSLQPEDVATYYCQQHYSTPWTFGGGTKVEIKR

SEQ ID NO: 48 - protein, 108 aa, Artificial Sequence
  huz-bmLC1E3.1_VK HYBRID SEQUENCE
DIQMTQSPSSLSASVGDRVTITCKASQDVSTAVAWYQQKPGKVPKLLIYWASTRHTGVPSRFSGSGSGTDYTLTISSLQAEDVATYYCQQHYSTPWTFGQGTKVEIKR

SEQ ID NO: 49 - protein, 214 aa, Artificial Sequence
  huz-LC1E3.1_LC HYBRID SEQUENCE
DIQMTQSPSSLSASVGDRVTITCKASQDVSTAVAWYQQKPGKVPKLLIYWASTRHTGVPSRFSGSGSGTDFTLTISSLQPEDVATYYCQQHYSTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 214 aa, Artificial Sequence
  huz-bmLC1E3.1_LC HYBRID SEQUENCE
DIQMTQSPSSLSASVGDRVTITCKASQDVSTAVAWYQQKPGKVPKLLIYWASTRHTGVPSRFSGSGSGTDYTLTISSLQAEDVATYYCQQHYSTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 118 aa, Artificial Sequence
  huz-HC1E3.1_VH HYRID SEQUENCE
QVQLVQSGAEVKKPGASVKVSCKASGYTFTEYTIHWVRQAPGQGLEWMGWFYPGSGSIKYNEKFRDRVTMTRDTSTSTVYMELSSLRSEDTAVYYCARLDRDYAMDYWGQGTTVTVSS

SEQ ID NO: 52 - protein, 118 aa, Artificial Sequence
  huz-bmHC1E3.1_VH HYBRID SEQUENCE
QVQLVQSGAEVKKPGASVKVSCKASGYTFTEYTIHWVKQRPGQGLEWMGWFYPGSGSIKYNEKFRDKVTMTADKSTSTVYMELSSLRSEDTAVYYCARLDRDYAMDYWGQGTTVTVSS

SEQ ID NO: 53 - protein, 118 aa, Artificial Sequence
  huz-HC2E1.1_VH HYBRID SEQUENCE
QVQLVQSGAEVKKPGASVKVSCKASGYTFTEYTINWVRQAPGQGLEWMGWFYPGSGSIKYNEKFRDRVTMTRDTSTSTVYMELSSLRSEDTAVYYCARLDRDYAMDYWGQGTTVTVSS

SEQ ID NO: 54 - protein, 118 aa, Artificial Sequence
  huz-HC1E3.1_IgG2 HYBRID SEQUENCE
QVQLVQSGAEVKKPGASVKVSCKASGYTFTEYTINWVKQRPGQGLEWMGWFYPGSGSIKYNEKFRDKVTMTADKSTSTVYMELSSLRSEDTAVYYCARLDRDYAMDYWGQGTTVTVSS

SEQ ID NO: 55 - protein, 444 aa, Artificial Sequence
  huz-bmHC1E3.1_IgG2 HYBRID SEQUENCE
QVQLVQSGAEVKKPGASVKVSCKASGYTFTEYTIHWVKQRPGQGLEWMGWFYPGSGSIKYNEKFRDKVTMTADKSTSTVYMELSSLRSEDTAVYYCARLDRDYAMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 56 - protein, 444 aa, Artificial Sequence; identical to SEQ ID NO: 55
  huz-bmHC1E3.1_IgG2 HYBRID SEQUENCE

SEQ ID NO: 57 - protein, 444 aa, Artificial Sequence
  huz-HC2E1.1_IgG2 HYBRID SEQUENCE
QVQLVQSGAEVKKPGASVKVSCKASGYTFTEYTINWVRQAPGQGLEWMGWFYPGSGSIKYNEKFRDRVTMTRDTSTSTVYMELSSLRSEDTAVYYCARLDRDYAMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 58 - protein, 444 aa, Artificial Sequence
  huz-bmHC2E1.1_IgG2 HYBRID SEQUENCE
QVQLVQSGAEVKKPGASVKVSCKASGYTFTEYTINWVKQRPGQGLEWMGWFYPGSGSIKYNEKFRDKVTMTADKSTSTVYMELSSLRSEDTAVYYCARLDRDYAMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 59 - protein, 448 aa, Artificial Sequence
  huz-HC1E3.1_eflsIgG1 HYBRID SEQUENCE
QVQLVQSGAEVKKPGASVKVSCKASGYTFTEYTIHWVRQAPGQGLEWMGWFYPGSGSIKYNEKFRDRVTMTRDTSTSTVYMELSSLRSEDTAVYYCARLDRDYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 448 aa, Artificial Sequence
  huz-bmHC1E3.1_ eflsIgG1 HYBRID SEQUENCE
QVQLVQSGAEVKKPGASVKVSCKASGYTFTEYTIHWVKQRPGQGLEWMGWFYPGSGSIKYNEKFRDKVTMTADKSTSTVYMELSSLRSEDTAVYYCARLDRDYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 448 aa, Artificial Sequence
  huz-HC2E1.1_ eflsIgG1 HYBRID SEQUENCE
QVQLVQSGAEVKKPGASVKVSCKASGYTFTEYTINWVRQAPGQGLEWMGWFYPGSGSIKYNEKFRDRVTMTRDTSTSTVYMELSSLRSEDTAVYYCARLDRDYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 448 aa, Artificial Sequence
  huz-bmHC2E1.1_ eflsIgG1 HYBRID SEQUENCE
QVQLVQSGAEVKKPGASVKVSCKASGYTFTEYTINWVKQRPGQGLEWMGWFYPGSGSIKYNEKFRDKVTMTADKSTSTVYMELSSLRSEDTAVYYCARLDRDYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 108 aa, ARTIFICIAL SEQUENCE
  HYBRID SEQUENCE
DIQMTQSPSSLSASVGDRVTITCRASENIYYSLAWYQQKPGKAPKLLIYNANSLEDGVPSRFSGSGSGTDFTLTISSLQPEDFATYYCKQAYDVPLTFGQGTKLEIKR

SEQ ID NO: 64 - protein, 108 aa, Homo sapiens
  MISC_FEATURE: >huz-bmLC2G2.2_VK
DIQMTQSPSSLSASVGDRVTITCRASENIYYSLAWYQQKPGKAPQLLIYNANSLEDGVPSRFSGSGSGTDYTLTISSLQPEDFATYFCKQAYDVPLTFGGGTKVEIKR

SEQ ID NO: 65 - protein, 214 aa, ARTIFICIAL SEQUENCE
  HYBRID SEQUENCE
DIQMTQSPSSLSASVGDRVTITCRASENIYYSLAWYQQKPGKAPKLLIYNANSLEDGVPSRFSGSGSGTDFTLTISSLQPEDFATYYCKQAYDVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 214 aa, ARTIFICIAL SEQUENCE
  HYBRID SEQUENCE
DIQMTQSPSSLSASVGDRVTITCRASENIYYSLAWYQQKPGKAPQLLIYNANSLEDGVPSRFSGSGSGTDYTLTISSLQPEDFATYFCKQAYDVPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 121 aa, ARTIFICIAL SEQUENCE
  HYBRID SEQUENCE
QVQLVQSGAEVKKPGASVKVSCKASGYTFTSYWMHWVRQAPGQGLEWMGNINPSNGGTNYNEKFKSRVTMTRDTSTSTVYMELSSLRSEDTAVYYCARSKNNYVAYYFDYWGQGTLVTVSS

SEQ ID NO: 68 - protein, 121 aa, ARTIFICIAL SEQUENCE
  HYBRID SEQUENCE
QVQLVQSGAEVKKPGASVKVSCKASGYTFTSYWMHWVRQAPGQGLERMGNINPSNGGTNYNEKFKSRVTMTVDKSSSTAYMELSSLRSEDTAVYYCARSKNNYVAYYFDYWGQGTLVTVSS

SEQ ID NO: 69 - protein, 447 aa, ARTIFICIAL SEQUENCE
  HYBRID SEQUENCE
QVQLVQSGAEVKKPGASVKVSCKASGYTFTSYWMHWVRQAPGQGLEWMGNINPSNGGTNYNEKFKSRVTMTRDTSTSTVYMELSSLRSEDTAVYYCARSKNNYVAYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 70 - protein, 447 aa, ARTIFICIAL SEQUENCE
  HYBRID SEQUENCE
QVQLVQSGAEVKKPGASVKVSCKASGYTFTSYWMHWVRQAPGQGLERMGNINPSNGGTNYNEKFKSRVTMTVDKSSSTAYMELSSLRSEDTAVYYCARSKNNYVAYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 71 - protein, 451 aa, ARTIFICIAL SEQUENCE
  HYBRID SEQUENCE
QVQLVQSGAEVKKPGASVKVSCKASGYTFTSYWMHWVRQAPGQGLEWMGNINPSNGGTNYNEKFKSRVTMTRDTSTSTVYMELSSLRSEDTAVYYCARSKNNYVAYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 451 aa, ARTIFICIAL SEQUENCE
  HYBRID SEQUENCE
QVQLVQSGAEVKKPGASVKVSCKASGYTFTSYWMHWVRQAPGQGLERMGNINPSNGGTNYNEKFKSRVTMTVDKSSSTAYMELSSLRSEDTAVYYCARSKNNYVAYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - DNA, 33 bp, Mus musculus
  misc_feature: ANTIBODY 2G2.2 LC CDR DNA SEQUENCE
cgagcaagtgagaacatttactacagtttagct

SEQ ID NO: 74 - DNA, 21 bp, Mus musculus
  misc_feature: ANTIBODY 2G2.2 LC CDR DNA SEQUENCE
aatgcaaacagcttggaagat

SEQ ID NO: 75 - DNA, 27 bp, Mus musculus
  misc_feature: ANTIBODY 2G2.2 LC CDR DNA SEQUENCE
aaacaggcttatgacgttcctctcacg

SEQ ID NO: 76 - DNA, 15 bp, Mus musculus
  misc_feature: ANTIBODY 2G2.2 HC CDR DNA SEQUENCE
agctactggatgcac

SEQ ID NO: 77 - DNA, 51 bp, Mus musculus
  misc_feature: ANTIBODY 2G2.2 HC CDR DNA SEQUENCE
aatattaatcctagcaatggtggtactaactacaatgagaagttcaagagt

SEQ ID NO: 78 - DNA, 36 bp, Mus musculus
  misc_feature: ANTIBODY 2G2.2 HC CDR DNA SEQUENCE
tcgaagaataactacgtggcgtactactttgactac

SEQ ID NO: 79 - protein, 11 aa, Mus musculus
  MISC_FEATURE: ANTIBODY 1H6.2 LC VARIABLE REGION CDR SEQUENCE
RASENIYSYLA

SEQ ID NO: 80 - protein, 7 aa, Mus musculus
  misc_feature: ANTIBODY 1H6.2 LC VARIABLE REGION CDR SEQUENCE
NAKTLAE

SEQ ID NO: 81 - protein, 9 aa, Mus musculus
  MISC_FEATURE: ANTIBODY 1H6.2 LC VARIABLE REGION CDR SEQUENCE
QHHYDTPPT

SEQ ID NO: 82 - protein, 5 aa, Mus musculus
  MISC_FEATURE: ANTIBODY 1H6.2 HC VARIABLE REGION CDR SEQUENCE
TYAMH

SEQ ID NO: 83 - protein, 19 aa, Mus musculus
  MISC_FEATURE: ANTIBODY 1H6.2 HC VARIABLE REGION CDR SEQUENCE
RIRSKSSNYATYYADSVKD

SEQ ID NO: 84 - protein, 11 aa, Mus musculus
  MISC_FEATURE: ANTIBODY 1H6.2 HC VARIABLE REGION CDR SEQUENCE
DGGYYVGYFDV

SEQ ID NO: 85 - protein, 234 aa, Mus musculus
  MISC_FEATURE: ANTIBODY 1H6.2 LIGHT CHAIN AMINO ACID SEQUENCE
MSVPTQVLGLLLLWLTGARCDIQMTQSPATLSASVGETVTITCRASENIYSYLAWYQQKQGKSPHLLVYNAKTLAEGVPSRFSGIGSGTQFSLKINSLQPEDFGSYYCQHHYDTPPTFGGGTNLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 86 - protein, 465 aa, Mus musculus
  MISC_FEATURE: ANTIBODY 1H6.2 HEAVY CHAIN AMINO ACID SEQUENCE
MVLGLKWVFFVVFYQGVHCEVQLVESGGGLVQPKGSLKLSCVASGFTFKTYAMHWVRQAPGKGLEWVARIRSKSSNYATYYADSVKDRFTISRDDSQSMLYLQMNNLKTEDTAMYYCVRDGGYYVGYFDVWGTGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 87 - protein, 11 aa, Mus musculus
  MISC_FEATURE: ANTIBODY TC3.2.1 LC VARIABLE REGION CDR SEQUENCE
RASENIYYSLA

SEQ ID NO: 88 - protein, 7 aa, Mus musculus
  MISC_FEATURE: ANTIBODY TC3.2.1 LC VARIABLE REGION CDR SEQUENCE
NANSLED

SEQ ID NO: 89 - protein, 9 aa, Mus musculus
  MISC_FEATURE: ANTIBODY TC3.2.1 LC VARIABLE REGION CDR SEQUENCE
KQAYDLPPT

SEQ ID NO: 90 - protein, 5 aa, Mus musculus
  MISC_FEATURE: ANTIBODY TC3.2.1 LC VARIABLE REGION CDR SEQUENCE
SSYIS

SEQ ID NO: 91 - protein, 17 aa, Mus musculus
  MISC_FEATURE: ANTIBODY TC3.2.1 HC VARIABLE REGION CDR SEQUENCE
WIYAGTGASTFNQKFTG

SEQ ID NO: 92 - protein, 12 aa, Mus musculus
  MISC_FEATURE: ANTIBODY TC3.2.1 HC VARIABLE REGION CDR SEQUENCE
KRAYYEGLYFAV

SEQ ID NO: 93 - protein, 234 aa, Mus musculus
  MISC_FEATURE: ANTIBODY TC3.2.1 LIGHT CHAIN AMINO ACID SEQUENCE
MSVPTQLLGLLLLWLTDAGCDIQMTQSPASLAASVGETVTITCRASENIYYSLAWYQQKQGKSPQLLIYNANSLEDGVPSRFSGSGSGTQYSMKINSMQPEDSATYFCKQAYDLPPTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 94 - protein, 470 aa, Mus musculus
  MISC_FEATURE: ANTIBODY TC3.2.1 HEAVY CHAIN AMINO ACID SEQUENCE
MEWNWVVLFLLSLTAGVYAQGQMQQSGPELVKPGASVKLSCKTSGFTFSSSYISWLKQKPGQSLEWIAWIYAGTGASTFNQKFTGKAQLTVDTTSSTAYMQFSSLTTEDSAIYYCARKRAYYEGLYFAVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 95 - protein, 11 aa, Mus musculus
  MISC_FEATURE: ANTIBODY 3B9.1 LC VARIABLE REGION CDR SEQUENCE
RASENIYYSLA

SEQ ID NO: 96 - protein, 7 aa, Mus musculus
  MISC_FEATURE: ANTIBODY 3B9.1 LC VARIABLE REGION CDR SEQUENCE
HTNTLED

SEQ ID NO: 97 - protein, 9 aa, Mus musculus
  MISC_FEATURE: KQAYDVPPT
KQAYDVPPT

SEQ ID NO: 98 - protein, 5 aa, Mus musculus
  MISC_FEATURE: ANTIBODY 3B9.1 HC VARIABLE REGION CDR SEQUENCE
SYWMH

SEQ ID NO: 99 - protein, 17 aa, Mus musculus
  MISC_FEATURE: ANTIBODY 3B9.1 HC VARIABLE REGION CDR SEQUENCE
NINPSSGVANYNEKFKN

SEQ ID NO: 100 - protein, 12 aa, Mus musculus
  MISC_FEATURE: ANTIBODY 3B9.1 HC VARIABLE REGION CDR SEQUENCE
SKSNYVAYYFDY

SEQ ID NO: 101 - protein, 234 aa, Mus musculus
  MISC_FEATURE: ANTIBODY 3B9.1 LIGHT CHAIN AMINO ACID SEQUENCE
MSVPTQLLGLLLLWLTDAGCDIQMTQSPASLAASVGETVTITCRASENIYYSLAWYQQKQGKSPQLLIYHTNTLEDGVPSRFSGSGSGTQYSMQINTMQPEDTATYFCKQAYDVPPTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 102 - protein, 464 aa, Mus musculus
  MISC_FEATURE: ANTIBODY 3B9.1 HEAVY CHAIN AMINO ACID SEQUENCE
MGWSCIILFLVAAATGVHSQVQLQQPGTELVKPGASVKLSCKASGYTFTSYWMHWVKQRPGQGLERIGNINPSSGVANYNEKFKNKATLTVDKSSSTAYMQLSSLTSEDSAVYYCARSKSNYVAYYFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 103 - protein, 11 aa, Mus musculus
  MISC_FEATURE: ANTIBODY 3C11.2 LC VARIABLE REGION CDR SEQUENCE
KASQDINSFLS

SEQ ID NO: 104 - protein, 7 aa, Mus musculus
  MISC_FEATURE: ANTIBODY 3C11.2 LC VARIABLE REGION CDR SEQUENCE
RANRLVD

SEQ ID NO: 105 - protein, 9 aa, Mus musculus
  MISC_FEATURE: ANTIBODY 3C11.2 LC VARIABLE REGION CDR SEQUENCE
LQYDEFPLT

SEQ ID NO: 106 - protein, 5 aa, Mus musculus
  MISC_FEATURE: ANTIBODY 3C11.2 HC VARIABLE REGION CDR SEQUENCE
SYWMH

SEQ ID NO: 107 - protein, 17 aa, Mus musculus
  MISC_FEATURE: ANTIBODY 3C11.2 HC VARIABLE REGION CDR SEQUENCE
YIDPSSRYTKYNLNFKD

SEQ ID NO: 108 - protein, 10 aa, Mus musculus
  MISC_FEATURE: ANTIBODY 3C11.2 HC VARIABLE REGION CDR SEQUENCE
YSNYVGAMDY

SEQ ID NO: 109 - protein, 236 aa, Mus musculus
  MISC_FEATURE: ANTIBODY 3C11.2 LIGHT CHAIN AMINO ACID SEQUENCE
MDMRTPAQFLGILLLWFPGIKCDIKMTQSPSSMYASLGERVTITCKASQDINSFLSWFQQKPGKSPKTLIYRANRLVDGVPSRFSGSGSGQDYSLTISSLEYEDMGIYYCLQYDEFPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 110 - protein, 474 aa, Mus musculus
  MISC_FEATURE: ANTIBODY 3C11.2 HEAVY CHAIN AMINO ACID SEQUENCE
MERHWIFLSLLSVIAGVHSQVQLQQSGAELAKPGASVRLSCKASGYSFSSYWMHWIKQRPGRGLEWIGYIDPSSRYTKYNLNFKDKATLTADKSSSTAYILLTSLTYEDSAVYYCATYSNYVGAMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRTPGK

SEQ ID NO: 111 - protein, 11 aa, Mus musculus
  MISC_FEATURE: ANTIBODY 1A11.2 LC VARIABLE REGION CDR SEQUENCE
KASQDINSYLS

SEQ ID NO: 112 - protein, 7 aa, Mus musculus
  MISC_FEATURE: ANTIBODY 1A11.2 LC VARIABLE REGION CDR SEQUENCE
RANRLVD

SEQ ID NO: 113 - protein, 9 aa, Mus musculus
  MISC_FEATURE: ANTIBODY 1A11.2 LC VARIABLE REGION CDR SEQUENCE
LQYDESPLT

SEQ ID NO: 114 - protein, 5 aa, Mus musculus
  MISC_FEATURE: ANTIBODY 1A11.2 HC VARIABLE REGION CDR SEQUENCE
SYWMH

SEQ ID NO: 115 - protein, 17 aa, Mus musculus
  MISC_FEATURE: ANTIBODY 1A11.2 HC VARIABLE REGION CDR SEQUENCE
YIDPSSGYTKYNQKFKD

SEQ ID NO: 116 - protein, 10 aa, Mus musculus
  MISC_FEATURE: ANTIBODY 1A11.2 HC VARIABLE REGION CDR SEQUENCE
YSNYVGAMDY

SEQ ID NO: 117 - protein, 236 aa, Mus musculus
  MISC_FEATURE: ANTIBODY 1A11.2 LIGHT CHAIN AMINO ACID SEQUENCE
MDMRTPAQFLGILLLWFPGIKCDIKMTQSPSSMYASLGERVTITCKASQDINSYLSWFQQKPGKSPKTLIYRANRLVDGVPSRFSGSGSGQDYSLTISSLEYEDMGIYYCLQYDESPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 118 - protein, 462 aa, Mus musculus
  MISC_FEATURE: ANTIBODY 1A11.2 HEAVY CHAIN AMINO ACID SEQUENCE
MERHWIFLSLLSVIAGVHSQVQLQQSGAEVAKPGASVKLSCKASGYTFTSYWMHWVNQRPGQGLEWIGYIDPSSGYTKYNQKFKDKATLTVDKSSSTAYMQLSSLTYEDSAVYYCATYSNYVGAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 119 - protein, 12 aa, Mus musculus
  MISC_FEATURE: ANTIBODY 1G12.1 LC VARIABLE REGION CDR SEQUENCE
SVTSNITSSNLH

SEQ ID NO: 120 - protein, 7 aa, Mus musculus
  MISC_FEATURE: ANTIBODY 1G12.1 LC VARIABLE REGION CDR SEQUENCE
GTSNLAS

SEQ ID NO: 121 - protein, 9 aa, Mus musculus
  MISC_FEATURE: ANTIBODY 1G12.1 LC VARIABLE REGION CDR SEQUENCE
QQWSSYPLT

SEQ ID NO: 122 - protein, 5 aa, Mus musculus
  MISC_FEATURE: ANTIBODY 1G12.1 HC VARIABLE REGION CDR SEQUENCE
GYYMN

SEQ ID NO: 123 - protein, 17 aa, Mus musculus
  MISC_FEATURE: ANTIBODY 1G12.1 HC VARIABLE REGION CDR SEQUENCE
EINPRTGGTTYNQKFKA

SEQ ID NO: 124 - protein, 12 aa, Mus musculus
  MISC_FEATURE: ANTIBODY 1G12.1 HC VARIABLE REGION CDR SEQUENCE
DYGSSPYWYFDV

SEQ ID NO: 125 - protein, 237 aa, Mus musculus
  MISC_FEATURE: ANTIBODY 1G12.1 LIGHT CHAIN AMINO ACID SEQUENCE
MDFHVQIFSVMLISVTVILSSGEIVLTQSPALMAASPGEKVTITCSVTSNITSSNLHWYQQKSQTSPKPWIYGTSNLASGVPVRFSGSGSGTSYSLIISSMEAEDAATYYCQQWSSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 126 - protein, 464 aa, Mus musculus
  MISC_FEATURE: ANTIBODY 1G12.1 HEAVY CHAIN AMINO ACID SEQUENCE
MGWNWIFILILTVTTGVHSEVQLQQSGPELVKPGASVKISCKASGYTFTGYYMNWVKQSPEKSLEWIGEINPRTGGTTYNQKFKAKATLTVDKSSSTAYMQLKSLTSEDSAVFYCARDYGSSPYWYFDVWGTGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 127 - protein, 11 aa, Mus musculus
  MISC_FEATURE: ANTIBODY 3B2.1 LC VARIABLE REGION CDR SEQUENCE
RTSENIYYSLA

SEQ ID NO: 128 - protein, 7 aa, Mus musculus
  MISC_FEATURE: ANTIBODY 3B2.1 LC VARIABLE REGION CDR SEQUENCE
NANSLED

SEQ ID NO: 129 - protein, 9 aa, Mus musculus
  MISC_FEATURE: ANTIBODY 3B2.1 LC VARIABLE REGION CDR SEQUENCE
KQAYDVPFT

SEQ ID NO: 130 - protein, 5 aa, Mus musculus
  MISC_FEATURE: ANTIBODY 3B2.1 HC VARIABLE REGION CDR SEQUENCE
GYFMN

SEQ ID NO: 131 - protein, 17 aa, Mus musculus
  MISC_FEATURE: ANTIBODY 3B2.1 HC VARIABLE REGION CDR SEQUENCE
RINPYNGDTFYNQKFKG

SEQ ID NO: 132 - protein, 11 aa, Mus musculus
  MISC_FEATURE: ANTIBODY 3B2.1 HC VARIABLE REGION CDR SEQUENCE
GAGALRNYFDH

SEQ ID NO: 133 - protein, 235 aa, Mus musculus
  MISC_FEATURE: ANTIBODY 3B2.1 LIGHT CHAIN AMINO ACID SEQUENCE
MSVPTQLLGLLLLWLTDAGCDIQMTQSPASLAASVGETVTITCRTSENIYYSLAWYQQKQGKSPQLLIYNANSLEDGVPSRFSGSGSGTQYSMKINSMQPEDTATYFCKQAYDVPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECC

SEQ ID NO: 134 - protein, 463 aa, Mus musculus
  MISC_FEATURE: ANTIBODY 3B2.1 HEAVY CHAIN AMINO ACID SEQUENCE
MGWSWIFLFLLSVTAGVFSEVQLQQSGPELVKPGDSVKISCKASGYSFTGYFMNWVMQSHGKSLEWIGRINPYNGDTFYNQKFKGKATLTVDKSSSTVHMELRSLTSEDSAVYYCAKGAGALRNYFDHWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 135 - protein, 11 aa, Mus musculus
  MISC_FEATURE: ANTIBODY 3G4.1 LC VARIABLE REGION CDR SEQUENCE
RASENIYSYLA

SEQ ID NO: 136 - protein, 7 aa, Mus musculus
  MISC_FEATURE: ANTIBODY 3G4.1 LC VARIABLE REGION CDR SEQUENCE
HAKTLAE

SEQ ID NO: 137 - protein, 9 aa, Mus musculus
  MISC_FEATURE: ANTIBODY 3G4.1 LC VARIABLE REGION CDR SEQUENCE
QHHYGTPPT

SEQ ID NO: 138 - protein, 5 aa, Mus musculus
  MISC_FEATURE: ANTIBODY 3G4.1 HC VARIABLE REGION CDR SEQUENCE
TYAMH

SEQ ID NO: 139 - protein, 19 aa, Mus musculus
  MISC_FEATURE: ANTIBODY 3G4.1 HC VARIABLE REGION CDR SEQUENCE
RIRSKSSNYATYYADSVKD

SEQ ID NO: 140 - protein, 11 aa, Mus musculus
  MISC_FEATURE: ANTIBODY 3G4.1 HC VARIABLE REGION CDR SEQUENCE
DGGYYEAWFAY

SEQ ID NO: 141 - protein, 234 aa, Mus musculus
  MISC_FEATURE: ANTIBODY 3G4.1 LIGHT CHAIN AMINO ACID SEQUENCE
MSVPTQVLGLLLLWLTGARCDIQMTQSPASLSASVGETVTITCRASENIYSYLAWYQQKQGKSPQLLVYHAKTLAEGVPSRFSGSGSGTQFSLKINSLQPEDFGSYYCQHHYGTPPTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 142 - protein, 467 aa, Mus musculus
  MISC_FEATURE: ANTIBODY 3G4.1 HEAVY CHAIN AMINO ACID SEQUENCE
MVLGLKWVFFVVFYQGVHCEVQLVESGGGLVQPKGSLKLSCAASGFTFNTYAMHWVRQAPGKGLEWVARIRSKSSNYATYYADSVKDRFTISRDDSQSMLYLQMNNLKTEDTAMYYCVRDGGYYEAWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGKGK

SEQ ID NO: 143 - protein, 11 aa, Mus musculus
  MISC_FEATURE: ANTIBODY 3H6.2 LC VARIABLE REGION CDR SEQUENCE
KASENVGTFVS

SEQ ID NO: 144 - protein, 7 aa, Mus musculus
  MISC_FEATURE: ANTIBODY 3H6.2 LC VARIABLE REGION CDR SEQUENCE
GASNRYT

SEQ ID NO: 145 - protein, 9 aa, Mus musculus
  MISC_FEATURE: ANTIBODY 3H6.2 LC VARIABLE REGION CDR SEQUENCE
GQSYSFPLT

SEQ ID NO: 146 - protein, 5 aa, Mus musculus
  MISC_FEATURE: ANTIBODY 3H6.2 HC VARIABLE REGION CDR SEQUENCE
TYWMH

SEQ ID NO: 147 - protein, 17 aa, Mus musculus
  MISC_FEATURE: ANTIBODY 3H6.2 HC VARIABLE REGION CDR SEQUENCE
NIDPSDSETHYNQKFKD

SEQ ID NO: 148 - protein, 12 aa, Mus musculus
  MISC_FEATURE: ANTIBODY 3H6.2 HC VARIABLE REGION CDR SEQUENCE
YYGSSYVGSMDY

SEQ ID NO: 149 - protein, 234 aa, Mus musculus
  MISC_FEATURE: ANTIBODY 3H6.2 LIGHT CHAIN AMINO ACID SEQUENCE
MESQTLVFISILLWLYGADGNIVMTQSPKSMSMSVGERVTLSCKASENVGTFVSWYQQKPEQSPKLLIYGASNRYTGVPDRFTGSGSATDFTLTISSVQAEDLADYHCGQSYSFPLTFGAGTKLEVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 150 - protein, 464 aa, Mus musculus
  MISC_FEATURE: ANTIBODY 3H6.2 HEAVY CHAIN AMINO ACID SEQUENCE
MGRSCIILFLVATATGVHSQVQLQQPGAELLRPGSSVKLSCKASGYTFTTYWMHWVKQRPIQGLEWIGNIDPSDSETHYNQKFKDKATLTVDKSSSTAYMQLSSLTSEDSAVYYCASYYGSSYVGSMDYWGQGTSVTVSSSKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 151 - protein, 16 aa, Homo sapiens
  MISC_FEATURE: ANTIBODY 16E6.1 LC VARIABLE REGION CDR SEQUENCE
KSSQSLLHSDGKTYLY

SEQ ID NO: 152 - protein, 7 aa, Homo sapiens
  MISC_FEATURE: ANTIBODY 16E6.1 LC VARIABLE REGION CDR SEQUENCE
EVSNRFS

SEQ ID NO: 153 - protein, 9 aa, Homo sapiens
  MISC_FEATURE: ANTIBODY 16E6.1 LC VARIABLE REGION CDR SEQUENCE
MQSIQLPLT

SEQ ID NO: 154 - protein, 5 aa, Homo sapiens
  MISC_FEATURE: ANTIBODY 16E6.1 HC VARIABLE REGION CDR SEQUENCE
SYNIH

SEQ ID NO: 155 - protein, 17 aa, Homo sapiens
  MISC_FEATURE: ANTIBODY 16E6.1 HC VARIABLE REGION CDR SEQUENCE
VIWYDGSNKYYVDSVKG

SEQ ID NO: 156 - protein, 10 aa, Homo sapiens
  MISC_FEATURE: ANTIBODY 16E6.1 HC VARIABLE REGION CDR SEQUENCE
DRNIDDAFDI

SEQ ID NO: 157 - protein, 113 aa, Homo sapiens
  MISC_FEATURE: ANTIBODY 16E6.1 LIGHT CHAIN VARIABLE DOMAIN AMINO ACID SEQUENCE
DIVMTQTPLSLSVTPGQPASISCKSSQSLLHSDGKTYLYWYLQKPGQPPQLLIYEVSNRFSGVPDRFSGSGSGTDFTLKISRVAAEDVGVYYCMQSIQLPLTFGGGTKVEIKR

SEQ ID NO: 158 - protein, 119 aa, Homo sapiens
  MISC_FEATURE: ANTIBODY 16E6.1 HEAVY CHAIN VARIABLE DOMAIN AMINO ACID SEQUENCE
QVQLVESGGGVVQPGRSLRLSCAASGFTFSSYNIHWVRQAPGKGLEWVAVIWYDGSNKYYVDSVKGRFTISRDNSKNTLYLQMNSLRAEDTAVYYCARDRNIDDAFDIWGQGTIVTVSS

(19) United States
(12) Patent Application Publication
(10) Pub. No.: US 2014/0271629 A1
(43) Pub. Date: Sep. 18, 2014

(54) CHRDL-1 ANTIGEN BINDING PROTEINS AND METHODS OF TREATMENT

(71) Applicant: Amgen Inc., Thousand Oaks, CA (US)

(72) Inventors: Kevin Christopher CORBIT, Thousand Oaks, CA (US); Christopher J.R. Paszty, Ventura, CA (US)

(21) Appl. No.: 14/207,397

(22) Filed: Mar. 12, 2014

Related U.S. Application Data
(60) Provisional application No. 61/784,988, filed on Mar. 14, 2013.
(60) Provisional application No. 61/861,721, filed on Aug. 2, 2013.

(51) Int. Cl. C07K 16/18

(52) U.S. Cl. CPC C07K 16/18
USPC 424/133.1; 424/139.1; 530/387.9; 530/387.3; 536/23.53; 435/320.1; 435/69.6; 435/331

(57) ABSTRACT

The present invention provides compositions and methods relating to or derived from antigen binding proteins to CHRDL-1. Human, humanized, and chimeric antibodies, as well as fragments and derivatives thereof are further contemplated. Other embodiments include nucleic acids encoding such antigen binding proteins, and fragments and derivatives thereof, as well as polypeptides, cells, methods of making and using antigen binding proteins, fragments and derivatives thereof.

### PRIORITY

This application claims benefit to U.S. Provisional Application No. 61/861,721 filed on Aug. 2, 2013, and U.S. Provisional Application No. 61/784,988, filed on Mar. 14, 2013, the contents of which are each hereby incorporated by reference in their entirety.

### SEQUENCE LISTING

The instant application contains a Sequence Listing which has been submitted in ASCII format via EFS-Web and is hereby incorporated by reference in its entirety. Said ASCII copy, created on Mar. 12, 2014, is named “A-1817-US-NP_SeqList03122014_ST25.txt” and is 72 bytes in size.

### FIELD OF THE INVENTION

The present disclosure relates to methods of treatment, as well as sequences encoding antigen binding proteins that bind to Chordin-like-1, as well as pharmaceutical compositions and methods of use thereof.

### BACKGROUND

The invention relates generally to brown fat, also known as brown adipose tissue (BAT).

In contrast to the energy storage provided by white adipose tissue (WAT), brown adipose tissue (BAT) expends energy by burning fats and sugars to produce heat instead of ATP. Nedergaard, J. & B. Cannon, Cell Metab. 11(4): 268-272 (2010). This unique action is due to the abundance of mitochondria within BAT and mediated by Uncoupling Protein 1 (UCP1). Feldmann, et al., Cell Metab. 9(2): 203-209 (2009). Recent evidence in mice and humans suggests that BAT has effects on sugar and lipid clearance. Specifically, active BAT normalizes triglyceride and glucose homeostasis in the setting of severe insulin resistance. Skarulis et al., J. Clin. Endocrinol. Metab. 95(1): 256-262 (2010). On the other hand, loss of BAT has been shown to induce metabolic syndrome. Lowell et al., Nature 366(6457): 740-742 (1993). A preponderance of BAT was also recently shown to be associated with extreme insulin sensitivity and resistant to high calorie weight gain. Padidela et al., Horm. Res. Paediatr., 77(4): 261-268 (2012). While these studies showed a role in metabolic homeostasis for BAT, interest was tempered by the thought that BAT was relegated to infants and lower mammals. In fact, BAT was considered vestigial in adult humans until multiple groups reported its identification. See Nedergaard et al., Am. J. Physiol. Endocrinol. Metab. 293(2): E444-452 (2007).

Two genetically distinct forms of BAT are present in mammals. The first is anatomically concentrated in the interscapular region and derives from a common skeletal muscle precursor. See Seale et al., Nature 454(7207): 961-967 (2008). The second appears within certain white adipose tissue (WAT) depots and has been termed “Brite” or “Beige” fat. See Klingenspor et al., Obes. Facts, 5(6): 890-896 (2012). Several inducers of both interscapular BAT and “Brite” cells have been identified, including Bone Morphogenetic Protein [Qian et al., PNAS 110(9): E798-807 (2013)], Fibroblast Growth Factor 21 [Wu et al., Sci. Transl. Med. 3(113): 113-126 (2011) and Kim et al., Nat. Med. 19(1): 83-92 (2013)], cold [Cypess et al., PNAS, 109(25): 10001-10005 (2012).], high-dose sympathomimetics [Carey et al., Diabetologia 56(1): 147-155 (2013)], insulin sensitizers [Digby et al., Diabetes, 47(1): 138-141 (1998)], and caloric restriction [Rothwell et al., Obes. Res. 5(6): 650-656 (1997) and Valle et al., Rejuvenation Res. 11(3): 597-604 (2008)].

Known inhibitors of BAT include aging [Rogers et al., Aging Cell, 11(6): 1074-1083 (2012)], the Fsp27 gene product [Nishino et al., J. Clin. Invest., 118(8): 2808-2821 (2008)], and TGF-B/SMAD3 signaling [Yadav et al., Cell Metab., 14(1): 67-79 (2011)].

Various genetic and/or gene expression profiling studies have been carried out in an effort to try and identify novel BAT regulators. For example, Seale et al. [Cell Metab, 6(1): 38-54 (2007)] identified a variety of BAT and adipose-specific genes, as well as Walden et al. [Am. J. Physiol. Endocrinol. Metab. 302(1): E19-31 (2012)]. Some identified efforts are set forth in TABLE 1 below (“NC” means no change).

TABLE 1

| | | | # Genes | CHRDL-1 | |
|---|---|---|---|---|---|
| Public Id | Species | Bioset name | changed | fold change | P Value |
| GSE7032 | Mouse | brown adipocytes culture 7 d _vs_{—} | 1434 | NC | N/A |
| | | preadipocyte culture 4 d | | | |
| GSE7032 | Mouse | brown adipocytes culture 7 d _vs_{—} | 2712 | NC | N/A |
| | | white adipocytes culture 7 d | | | |
| GSE7032 | Mouse | brown preadipocytes culture | 2145 | NC | N/A |
| | | 4 d _vs_ white preadipocytes | | | |
| | | culture 4 d | | | |
| GSE7032 | Mouse | white adipocytes culture 7 d _vs_{—} | 1894 | NC | N/A |
| | | preadipocyte culture 4 d | | | |
| GSE8505 | Mouse | Adipocytes of Subcutaneous | 1234 | NC | N/A |
| | | adipose tissue _vs_ of | | | |
| | | Intraabdominal adipose tissue | | | |
| GSE8505 | Mouse | Intraabdominal adipose tissue - | 4599 | NC | N/A |
| | | adipocytes _vs_ Stromo-vascular | | | |
| | | fraction | | | |
| GSE8505 | Mouse | Stromo-vascular fraction of | 1932 | NC | N/A |
| | | Subcutaneous adipose tissue _vs_{—} | | | |
| | | of Intraabdominal adipose tissue | | | |
| GSE8505 | Mouse | Subcutaneous adipose tissue - | 4538 | NC | N/A |
| | | adipocytes _vs_ Stromo-vascular | | | |
| | | fraction | | | |
| GSE4656 | Mouse | Brown fat from glycerol kinase | 2980 | −1.88 | 0.0029 |
| | | knockout mice _vs_ wildtype | | | |
| GSE9131 | Mouse | Epididymal fat pads from | 5234 | −3.36 | 0.0118 |
| | | lipodystrophic Pparg-Idi −/+ | | | |
| | | mice _vs_ wild-type | | | |
| GSE9130 | Mouse | Epididymal fat pads from aP2- | 3853 | −8.1 | 0.0014 |
| | | nSrebp1c transgenic mice _vs_{—} | | | |
| | | wild-type | | | |
| GSE5041 | Mouse | Differentiated brown fat cells | 4077 | −1.65 | 0.0269 |
| | | from PGC-1alpha KO mice | | | |
| | | dibutyryl cAMP treated 4 hr _vs_{—} | | | |
| | | untreated | | | |
| GSE5041 | Mouse | Differentiated brown fat cells | 3001 | −1.72 | 0.024 |
| | | from PGC-1alpha knockout _vs_{—} | | | |
| | | wildtype | | | |
| GSE5041 | Mouse | Differentiated brown fat cells | 3101 | NC | N/A |
| | | from wildtype mice dibutyryl | | | |
| | | cAMP treated 4 hr _vs_ untreated | | | |
| GSE5042 | Mouse | Differentiated brown fat cells | 869 | NC | N/A |
| | | from PGC-1 alpha knockout mice - | | | |
| | | PGC-1beta-siRNA _vs_ control- | | | |
| | | siRNA | | | |
| GSE5042 | Mouse | Differentiated brown fat cells | 2094 | −1.61 | 0.0207 |
| | | from PGC-1alpha knockout | | | |
| | | mice _vs_ wildtype | | | |
| GSE2674 | Mouse | White adipose tissue + beta3- | 1550 | NC | N/A |
| | | adrenergic receptor agonist | | | |
| | | 3 h _vs_ saline | | | |
| GSE7026 | Mouse | White adipose tissue from Alox5 | 1792 | NC | N/A |
| | | knockout mouse _vs_ wild type | | | |
| GSE7029 | Mouse | White adipose tissue from mice | 905 | NC | N/A |
| | | overexpressing ZFP90 _vs_{—} | | | |
| | | wildtype | | | |
| E-MARS-9 | Mouse | Brown adipose tissue from HSL | 1869 | NC | N/A |
| | | knock-out mouse _vs_ wild type | | | |
| E-MARS-9 | Mouse | White adipose tissue from HSL | 1573 | NC | N/A |
| | | knock-out mouse _vs_ wild type | | | |
| E-MARS-8 | Mouse | Brown adipose tissue from ATGL | 6873 | NC | N/A |
| | | knock-out mice _vs_ wild type | | | |
| E-MARS-8 | Mouse | White adipose tissue from ATGL | 1074 | NC | N/A |
| | | knock-out mice _vs_ wild type | | | |
| GSE8044 | Mouse | Mus musculus brown adipose | 10,283 | −18.5 | 0.0032 |
| | | tissue _vs_ white adipose tissue | | | |
| E-MEXP-872 | Human | Adipose tissue-derived human | 4058 | 32 | 6.10E−05 |
| | | stem cells _vs_ hematopoietic | | | |
| | | SCs | | | |
| GSE2510 | Human | Abdominal subcutaneous | 1698 | NC | N/A |
| | | preadipocytes from non-diabetic | | | |
| | | obese Pima Indian donors _vs_{—} | | | |
| | | lean | | | |
| GSE13585 | Mouse | Brown adipose tissue from KRAP | 958 | NC | N/A |
| | | KO mouse _vs_ wildtype | | | |
| GSE11396 | Mouse | Subcutaneous fat of mouse with | 1465 | NC | N/A |
| | | GHR knockout _vs_ wildtype | | | |
| GSE13432 | Mouse | White adipose tissue of mouse | 7740 | −2.95 | 0.0015 |
| | | housed at 4 degrees 1 wk _vs_{—} | | | |
| | | room temperature | | | |
| GSE13432 | Mouse | White adipose tissue of mouse | 6384 | −1.86 | 0.0007 |
| | | housed at 4 degrees 5 wk _vs_{—} | | | |
| | | room temperature | | | |
| GSE4692 | Mouse | Adipose tissue from mouse on | 240 | NC | N/A |
| | | high fat diet 4 wk with high | | | |
| | | weight gain _vs_ low weight gain | | | |
| GSE7027 | Mouse | Mouse white adipose tissue + | 450 | NC | N/A |
| | | rosiglitazone _vs_ vehicle control | | | |
| GSE10785 | Mouse | Adipose tissue from 10 wk old B6 | 8327 | −2.98 | 0.0001 |
| | | mice - obese _vs_ lean | | | |
| GSE10785 | Mouse | Adipose tissue from 10 wk old | 6915 | −1.71 | 0.0195 |
| | | BTBR mice - obese _vs_ lean | | | |
| GSE10785 | Mouse | Adipose tissue from 4 wk old B6 | 7320 | −2.58 | 0.0016 |
| | | mice - obese _vs_ lean | | | |
| GSE10785 | Mouse | Adipose tissue from 4 wk old | 6905 | −2.49 | 1.30E−07 |
| | | BTBR mice - obese _vs_ lean | | | |
| GSE10785 | Mouse | Adipose tissue from lean B6 mice - | 4126 | NC | N/A |
| | | 10 wk old _vs_ 4 wk | | | |
| GSE10785 | Mouse | Adipose tissue from lean BTBR | 3886 | 1.45 | 0.0006 |
| | | mice - 10 wk old _vs_ 4 wk | | | |
| GSE10785 | Mouse | Adipose tissue from obese B6 | 6687 | NC | N/A |
| | | mice - 10 wk old _vs_ 4 wk | | | |
| GSE10785 | Mouse | Adipose tissue from obese BTBR | 8442 | 2.12 | 0.0065 |
| | | mice - 10 wk old _vs_ 4 wk | | | |
| GSE10246 | Mouse | Brown adipose _vs_ white | 5855 | −3.38 | 0.0282 |
| | | adipose | | | |
| GSE15524 | Human | Adipose tissue from lean | 520 | NC | N/A |
| | | individual - subcutaneous _vs_{—} | | | |
| | | omental | | | |
| GSE15524 | Human | Adipose tissue from obese | 365 | NC | N/A |
| | | individual - subcutaneous _vs_{—} | | | |
| | | omental | | | |
| GSE15524 | Human | Omental adipose tissue - obese | 1210 | NC | N/A |
| | | individual _vs_ lean | | | |
| GSE15524 | Human | Subcutaneous adipose tissue - | 122 | NC | N/A |
| | | obese individual _vs_ lean | | | |
| GSE16106 | Mouse | Brown adipose tissue from | 2149 | NC | N/A |
| | | C57BL mice - PKGI null vs | | | |
| | | wildtype | | | |
| GSE19954 | Mouse | Epididymal fat tissue from mice | 1897 | NC | N/A |
| | | on high fat diet _vs_ control diet | | | |
| GSE19811 | Mouse | Subcutaneous abdominal adipose | 2508 | −1.29 | 0.0329 |
| | | tissue of HIV-infected patients on | | | |
| | | antiretrovirals _vs_ of controls | | | |
| GSE22506 | Mouse | Gonadal white adipose of GHSR | 557 | NC | N/A |
| | | KO mice + intraperitoneal | | | |
| | | injection of UAG _vs_ of saline | | | |
| | | control | | | |
| GSE20950 | Human | Adipose tissue from obese insulin | 358 | NC | N/A |
| | | resistant men - omental _vs_{—} | | | |
| | | subcutaneous | | | |
| GSE20950 | Human | Adipose tissue from obese insulin | 3237 | NC | N/A |
| | | resistant women - omental _vs_{—} | | | |
| | | subcutaneous | | | |
| GSE20950 | Human | Adipose tissue from obese insulin | 905 | NC | N/A |
| | | sensitive men - omental _vs_{—} | | | |
| | | subcutaneous | | | |
| GSE20950 | Human | Adipose tissue from obese insulin | 6738 | −1.3 | 0.0032 |
| | | sensitive women - omental _vs_{—} | | | |
| | | subcutaneous | | | |
| GSE22429 | Human | Adipose-derived stem cells | 1436 | NC | N/A |
| | | differentiated 14 d _vs_{—} | | | |
| | | undifferentiated | | | |
| GSE5090 | Human | Omental adipose tissue from | 320 | 1.34 | 0.0028 |
| | | polycystic ovary syndrome | | | |
| | | patients _vs_ control individuals | | | |
| GSE10478 | Mouse | Fat from Diabetic NOD | 1085 | NC | N/A |
| | | mice _vs_ control | | | |
| GSE17923 | Mouse | White adipose tissue of 6 mo old | 1806 | NC | N/A |
| | | female C57B16 mice - KSR2 | | | |
| | | KO _vs_ wildtype | | | |
| GSE22693 | Mouse | Gonadal white adipose tissue of | 1109 | −2.77 | 0.0069 |
| | | 3 mo old male mice - Fsp27 | | | |
| | | KO _vs_ WT | | | |
| GSE15773 | Human | Omental adipose tissue from | 1057 | NC | N/A |
| | | insulin resistant obese | | | |
| | | patients _vs_ insulin sensitive | | | |
| GSE15773 | Human | Subcutaneous adipose tissue from | 1167 | NC | N/A |
| | | insulin resistant obese | | | |
| | | patients _vs_ insulin sensitive | | | |
| GSE23337 | Mouse | Adipose tissue of ob/ob mice on | 2694 | −1.75 | 0.0211 |
| | | 28 d diet of conjugated linoleic | | | |
| | | acid - high level _vs_ low level | | | |
| GSE24637 | Mouse | Adipose tissue of TALLYHO × | 1094 | NC | N/A |
| | | C57BL6 F2 Type 2 diabetic mice | | | |
| | | at 24 wk - high triglycerides _vs_{—} | | | |
| | | low | | | |
| GSE20571 | Human | Abdominal subcutaneous adipose | 667 | NC | N/A |
| | | tissues - gastrointestinal cancer | | | |
| | | patients with cachexia _vs_{—} | | | |
| | | without | | | |
| GSE26339 | Human | Isolated adipocytes of cardiac | 2121 | NC | N/A |
| | | surgery patients - | | | |
| | | subcutaneous _vs_ pericardial | | | |
| GSE26339 | Human | Pericardial adipose tissue of | 4827 | NC | N/A |
| | | cardiac surgery patients - isolated | | | |
| | | adipocytes _vs_ whole tissue | | | |
| GSE26339 | Human | Subcutaneous adipose tissue of | 7753 | −1.43 | 0.023 |
| | | cardiac surgery patients - isolated | | | |
| | | adipocytes _vs_ whole tissue | | | |
| GSE26339 | Human | Whole adipose tissue of cardiac | 3124 | NC | N/A |
| | | surgery patients - | | | |
| | | subcutaneous _vs_ pericardial | | | |
| GSE21535 | Cow | Subcutaneous white adipose | 1841 | −1.28 | 0.0481 |
| | | tissues from 14 d postpartum | | | |
| | | cows _vs_ 30 d prepartum cows | | | |
| GSE15822 | Mouse | Brown fat from 12 wk old mice | 3399 | NC | N/A |
| | | after 6 wk on high fat diet _vs_{—} | | | |
| | | standard breeding diet | | | |
| GSE15822 | Mouse | Gonadal fat from 12 wk old mice | 2660 | NC | N/A |
| | | after 6 wk on high fat diet _vs_{—} | | | |
| | | standard breeding diet | | | |
| GSE15822 | Mouse | Inguinal fat from 12 wk old mice | 3759 | NC | N/A |
| | | after 6 wk on high fat diet _vs_{—} | | | |
| | | standard breeding diet | | | |
| GSE15822 | Mouse | Mesenteric fat from 12 wk old mice | 1745 | NC | N/A |
| | | after 6 wk on high fat diet _vs_{—} | | | |
| | | standard breeding diet | | | |
| GSE15822 | Mouse | Perirenal fat from 12 wk old mice | 4266 | NC | N/A |
| | | after 6 wk on high fat diet _vs_{—} | | | |
| | | standard breeding diet | | | |
| GSE15822 | Mouse | Subcutaneous fat from 12 wk old mice | 3475 | NC | N/A |
| | | after 6 wk on high fat diet _vs_{—} | | | |
| | | standard breeding diet | | | |
| GSE27309 | Mouse | Brown adipose tissue of 1 yr old | 375 | −1.21 | 0.0218 |
| | | mice - SIRT3 null _vs_ wildtype | | | |
| GSE23736 | Mouse | White adipose tissue of mice on a | 6826 | 4.2 | 9.30E−05 |
| | | high fat diet 6 wk - RORa | | | |
| | | null _vs_ wildtype littermates | | | |
| GSE28598 | Mouse | White adipose tissue of Smad3 | 2074 | 1.27 | 0.0346 |
| | | KO mice on high fat (55%) | | | |
| | | diet _vs_ regular diet | | | |
| GSE28598 | Mouse | White adipose tissue of Smad3 | 1434 | NC | N/A |
| | | wildtype mice on high fat (55%) | | | |
| | | diet _vs_ regular diet | | | |
| GSE28598 | Mouse | White adipose tissue of mice | 3442 | 1.86 | 0.0286 |
| | | obese from high fat diet + 8 wk | | | |
| | | anti-TGFB1 antibody _vs_ IgG | | | |
| | | control | | | |
| GSE28598 | Mouse | White adipose tissue of mice on | 1451 | NC | N/A |
| | | high fat (55%) diet - Smad3 | | | |
| | | KO _vs_ wildtype | | | |
| GSE28598 | Mouse | White adipose tissue of mice on | 2994 | −1.76 | 0.0282 |
| | | regular diet - Smad3 KO vs | | | |
| | | wildtype | | | |
| GSE29207 | Human | Adipose tissue-derived stem cells | 2595 | 1.22 | 0.0454 |
| | | in adipogenic medium 4 d - | | | |
| | | expressing mir-30a _vs_ negative | | | |
| | | control | | | |
| GSE29207 | Human | Adipose tissue-derived stem cells | 2175 | 1.31 | 0.0158 |
| | | in adipogenic medium 4 d - | | | |
| | | expressing mir-30d _vs_ negative | | | |
| | | control | | | |
| GSE28440 | Mouse | Epididymal adipose tissue from | 3214 | NC | N/A |
| | | mice fed high fat diet 13 wk _vs_{—} | | | |
| | | normal diet | | | |
| GSE28440 | Mouse | Inguinal subcutaneous adipose | 481 | NC | N/A |
| | | tissue from mice fed high fat diet | | | |
| | | 13 wk _vs_ normal diet | | | |
| GSE28440 | Mouse | Interscapular brown adipose | 668 | NC | N/A |
| | | tissue from mice fed high fat diet | | | |
| | | 13 wk _vs_ normal diet | | | |
| GSE28440 | Mouse | Mice fed high fat diet 13 wk - | 2773 | −2.19 | 0.0091 |
| | | perivascular adipose tissue _vs_{—} | | | |
| | | inguinal subcutaneous adipose | | | |
| | | tissue | | | |
| GSE28440 | Mouse | Mice fed normal diet 13 wk - | 4320 | −5.66 | 0.0006 |
| | | perivascular adipose tissue _vs_{—} | | | |
| | | epididymal adipose tissue | | | |
| GSE25205 | Mouse | Epididymal white adipose tissue | 286 | NC | N/A |
| | | of mice fed 16 wk high-fat diet - | | | |
| | | ASC null _vs_ wildtype | | | |
| GSE25205 | Mouse | Epididymal white adipose tissue | 4311 | 2.02 | 0.0295 |
| | | of mice fed 16 wk high-fat diet - | | | |
| | | Casp1 null _vs_ wildtype | | | |
| GSE23506 | Human | Subcutaneous adipose tissue from | 3031 | 1.81 | 0.0203 |
| | | obese young adults - African | | | |
| | | American _vs_ Hispanic | | | |
| GSE27666 | Human | Adipose tissue of Finnish males | 1501 | NC | N/A |
| | | with APOB region variant | | | |
| | | rs7575840 - rare T _vs_ common | | | |
| | | G allele | | | |
| GSE30929 | Human | Liposarcoma subtypes - | 3169 | −4.56 | 3.00E−08 |
| | | dedifferentiated _vs_ well- | | | |
| | | differentiated | | | |
| GSE32316 | Mouse | Brown adipose of ob/ob mice 4 d | 1403 | −1.23 | 0.042 |
| | | after IP injection with 1 mg/kg | | | |
| | | agonistic anti-FGFR1 _vs_ IgG | | | |
| | | controls | | | |
| GSE24425 | Human | Mediastinal adipose tissue coronary | 1994 | −1.37 | 0.006 |
| | | artery disease patient _vs_{—} | | | |
| | | subcutaneous adipose tissue | | | |
| GSE30116 | Mouse | Adipose progenitors from | 741 | NC | N/A |
| | | AdipoTrak mice treated 2 mo with | | | |
| | | rosiglitazone (in chow) _vs_{—} | | | |
| | | untreated | | | |
| GSE29411 | Human | Adipose tissue at time of bariatric | 872 | NC | N/A |
| | | surgery - Omental _vs_{—} | | | |
| | | subcutaneous adipose | | | |
| | | tissue_GPL7020 | | | |
| GSE29411 | Human | Subcutaneous adipose tissue - | 672 | NC | N/A |
| | | 4 mo post bariatric surgery _vs_ at | | | |
| | | time of surgery_GPL7020 | | | |
| GSE35431 | Mouse | Gonadal adipose tissue of 12 wk | 3089 | NC | N/A |
| | | old mice - caveolin-1 null _vs_{—} | | | |
| | | wildtype | | | |
| GSE35378 | Mouse | Adipose tissue - adipose-specific | 112 | NC | N/A |
| | | Glut4 KO _vs_ genetic control | | | |
| GSE35411 | Human | Subcutaneous adipose tissue after | 2279 | NC | N/A |
| | | maintained weight loss (6 mo) _vs_{—} | | | |
| | | before weight loss diet | | | |
| GSE22651 | Human | Adipose tissue _vs_ BM-MSCs | 3707 | 5.1 | 0.0104 |
| | | CD105+CD34−SC31− | | | |
| GSE13070 | Human | Adipose tissue TZD treated for | 1677 | NC | N/A |
| | | 3 mo - TZD non-responders _vs_{—} | | | |
| | | responders | | | |
| GSE13070 | Human | Adipose tissue TZD treated for | 3999 | 1.34 | 0.0105 |
| | | 3 mo - insulin resistant | | | |
| | | subjects _vs_ sensitive | | | |
| GSE13070 | Human | Adipose tissue TZD treated for | 2161 | NC | N/A |
| | | 3 mo _vs_ before TZD | | | |
| GSE13070 | Human | Adipose tissue before TZD - | 6910 | 1.73 | 0.0019 |
| | | insulin resistant subjects _vs_{—} | | | |
| | | sensitive | | | |
| GSE32095 | Mouse | Epididymal adipose tissue from | 9871 | −2.92 | 0.007 |
| | | GPR120 KO mice - high fat | | | |
| | | diet _vs_ normal diet control | | | |
| GSE32095 | Mouse | Epididymal adipose tissue from | 4751 | −5.53 | 0.0034 |
| | | mice on high fat diet - GPR120 | | | |
| | | KO _vs_ wildtype | | | |
| GSE32095 | Mouse | Epididymal adipose tissue from | 2432 | 1.72 | 0.0432 |
| | | mice on normal diet - GPR120 | | | |
| | | KO _vs_ wildtype | | | |
| GSE32095 | Mouse | Epididymal adipose tissue from | 9742 | 5.95 | 0.0009 |
| | | wildtype mice - high fat diet _vs_{—} | | | |
| | | normal diet control | | | |
| GSE28005 | Human | Adipose tissue before high fat | 4140 | NC | N/A |
| | | diet - overweight _vs_ lean | | | |
| GSE28005 | Human | Adipose tissue from lean patients - | 1153 | NC | N/A |
| | | high fat diet 56 d _vs_ before | | | |
| | | diet | | | |
| GSE28005 | Human | Adipose tissue from overweight | 521 | NC | N/A |
| | | patients - high fat diet 56 d _vs_{—} | | | |
| | | before diet | | | |
| GSE31646 | Mouse | Epididymal fat - Kinin B1 | 539 | NC | N/A |
| | | receptor knockout _vs_ wildtype | | | |
| | | controls | | | |
| GSE26637 | Mouse | Adipose + 3 hr hyperinsulinemic | 4440 | 1.32 | 0.0173 |
| | | euglycemic clamp - obese insulin | | | |
| | | resistant mice _vs_ lean sensitive | | | |
| GSE26637 | Mouse | Adipose from fasted lean insulin | 3202 | NC | N/A |
| | | sensitive mice - 3 hr | | | |
| | | hyperinsulinemic euglycemic | | | |
| | | clamp _vs_ before | | | |
| GSE26637 | Mouse | Adipose from fasted mice - obese | 1937 | NC | N/A |
| | | insulin resistant _vs_ lean insulin | | | |
| | | sensitive | | | |
| GSE26637 | Mouse | Adipose from fasted obese insulin | 1033 | NC | N/A |
| | | resistant mice - 3 hr | | | |
| | | hyperinsulinemic euglycemic | | | |
| | | clamp _vs_ before | | | |
| GSE35011 | Mouse | SV cells of white adipose tissue + | 2265 | NC | N/A |
| | | control vector - treated with | | | |
| | | rosiglitazone _vs_ untreated | | | |
| GSE35011 | Mouse | SV cells of white adipose tissue | 2163 | −1.24 | 0.0271 |
| | | treated with rosiglitazone - | | | |
| | | expressing PRDM16 _vs_ control | | | |
| | | vector | | | |
| GSE31692 | Mouse | Epidydimal white adipose tissue | 3742 | NC | N/A |
| | | of mice on control diet - FGF1 | | | |
| | | KO _vs_ wildtype | | | |
| GSE31692 | Mouse | Epidydimal white adipose tissue | 4872 | NC | N/A |
| | | of wildtype mice - high fat | | | |
| | | diet _vs_ control diet | | | |
| GSE39006 | Cow | Adipose from 26 mo old Holstein | 1312 | −2.79 | 0.0093 |
| | | steer - intramuscular _vs_{—} | | | |
| | | subcutaneous | | | |
| GSE39006 | Cow | Adipose from 26 mo old Japanese | 2258 | −2.73 | 0.0227 |
| | | Black steer - intramuscular _vs_{—} | | | |
| | | subcutaneous | | | |
| GSE39313 | Mouse | Epididymal white adipose of | 4985 | 1.28 | 0.0016 |
| | | C57BL/6J mice fasted 24 hr _vs_{—} | | | |
| | | fed ad libitum | | | |
| GSE39313 | Mouse | Epididymal white adipose of | 851 | NC | N/A |
| | | C57BL/6J mice fed ad libitum - | | | |
| | | FGF21-transgenic _vs_ wildtype | | | |
| | | controls | | | |
| GSE30247 | Mouse | White adipose tissue from mice | 5843 | −1.73 | 0.0279 |
| | | on high fat diet 8wk - conditional | | | |
| | | SIRT1 KO _vs_ wildtype | | | |
| GSE30247 | Mouse | White adipose tissue from mice | 5421 | −1.39 | 0.0203 |
| | | on low fat diet 8 wk - conditional | | | |
| | | SIRT1 KO _vs_ wildtype | | | |
| GSE30247 | Mouse | White adipose tissue wildtype | 8812 | −1.71 | 0.0029 |
| | | SIRT1 - high fat diet 8 wk _vs_{—} | | | |
| | | low fat diet | | | |
| GSE39562 | Mouse | Adipose cells lines treated with | 3591 | NC | N/A |
| | | 10 mM forskolin 4hr - from | | | |
| | | interscapular depots _vs_ inguinal | | | |
| | | depots | | | |

It will be appreciated that the expression of at least more than a hundred, and even thousands of genes, were changed in each of these individual studies, making the identification of biologically relevant targets not possible from the simple gene expression level outputs of microarray experiments or standard gene expression profiling (e.g., Q-PCR) experiments. In addition, determining what genes are simply markers of adipose tissue/cells versus functional biological effectors cannot be done from these types of data.

Additional work on BAT can be found in Bartell et al., Nat. Med. 17(2): 200-205 (2011), Cypess et al., N. Engl. J. Med. 360(15): 1509-1517 (2009), Saito et al., Diabetes 58(7): 1526-1531 (2009) and van Marken Lichtenbelt et al., N. Engl. J. Med. 360(15): 1500-1508 (2009).

Chordin-like-1 (also referred to herein as “Chordin-like 1”, “CHRDL-1”, “Chrdl-1”, “CHRDL1”, “Chrdl1”, “Neuralin”, “Neuralin-1”, “Neurogenesin-1”, or “Ventroptin”, is a secreted Bone Morphogenetic Protein (BMP) inhibitor. See e.g., Nakayama et al., Dev. Biol. 232(2): 372-387 (2001), Sakuta et al., Science 293(5527): 111-115 (2001), Larman et al., J. Am. Soc. Nephrol. 20(5): 1020-1031 (2009), and Webb et al., Am. J. Hum. Genet. 90(2): 247-259 (2012). Chordin-like-1 is abbreviated herein as “CHRDL-1”.

It will be appreciated that human Chordin-like-1 is herein referred to by the prefix “h” or “hu” affixed to any of the chordin-like-1 aliases listed above (e.g., “hCHRDL-1” or “huCHRDL-1”), and murine Chordin-like-1 is herein referred to by the prefix “m” or “mu” affixed to any of the chordin-like-1 aliases listed above (e.g., “mCHRDL-1”).

CHRDL-1 is highly enriched in white adipose tissue. The only previously suggested role for CHRDL-1 in adipose tissue was from a microarray study comparing a parental mouse mesenchymal stem cell line (C3H10T1/2) to a sub-clone (A33) with a high capacity for adipogenic differentiation. See Bowers et al., PNAS USA 103(35): 13022-13027 (2006). The authors of this study hypothesized that CHRDL-1 was involved in neuronal innervations of adipose tissue. However, a second study comparing the exact same two cell lines failed to identify CHRDL-1 by microarray [Bowers et al., Cell Cycle 7(9): 1191-1196 (2008)].

Bone Morphogenetic Protein 4 (BMP4) is known to promote the formation of fat cells (adipocytes) from precursor fibroblasts called pre-adipocytes in a process called adipogenesis. However, pre-adipocytes cultured from patients with hypertrophic obesity, a condition characterized by the presence of physically large adipocytes, do not undergo adipogenesis in response to BMP4. It was reported that these refractive hypertrophic pre-adipocytes increase their expression of BMP4 (Gustafson et al. 2012). A survey of six known BMP inhibitors by quantitative polymerase chain reaction (qPCR) demonstrated in increase in both NOGGIN and CHRDL-1 in hypertrophic pre-adipocytes as compared to pre-adipocytes cultured from lean individuals [Ulf Smith, Lundberg Laboratory, Univ. of Gothenburg, Sweden, 2012 American Diabetes Assoc. mtg. (June 8-12, Philadelphia)]. The functional relevance, if any, of these increases is unknown and may simply reflect a cellular response to increased levels of BMP4.

Splice variants of human and mouse CHRDL-1 have been identified. See e.g., http://www.ensembl.org/Mus_musculus/Gene/Summary?g=ENSMUSG00000031283;r=X:143285674-143394262 (Mouse: 4 total transcripts). 7 Human transcripts can be found at http://www.ensembl.org/Homo_sapiens/Gene/Summary?g=ENSG00000101938;r=X:109917084-110039286. The contents of these splice variants are hereby incorporated by reference in their entirety.

### SUMMARY OF THE INVENTION

In accordance with the present invention, the metabolic role of CHRDL-1 was elucidated by generating knock-out mice. When fed a diet of normal chow, no gross differences between wild-type (WT) and CHRDL-1 knockout (KO) mice was identified (e.g., body weight, glucose tolerance). To identify whether CHRDL-1 plays a role in pathological metabolism WT and KO, mice were fed a high-fat diet (HFD), an art-known model of human obesity and glucose intolerance in rodents. Under these conditions, KO mice gained ˜23% less weight than WT animals and maintained glucose tolerance. Histologically, KO mice retained and had more BAT following HFD. These results, along with the additional experimental protocols and data described herein demonstrate that CHRDL-1 is a physiological inhibitor of BAT. It is thus desirable to obtain inhibitors of CHRDL-1 and/or CHRDL-1 activity to treat a variety of adipose, glucose, cardiac, and related diseases and disorders. Experimental research provides support for inhibiting an activity of CHRDL-1 as a means to treat diabetes (including type 2 diabetes), diabetes-related disorders including but not limited to diabetic retinopathy and diabetic nephropathy, as well as obesity, dyslipidemia, and other metabolic conditions or disorders in humans. Additionally, inhibiting CHRDL-1 can be beneficial in treating kidney disease, including, but not limited to, acute kidney injury, chronic kidney disease, and polycystic kidney disease, as well as various types of heart disease, including coronary heart disease, as well as related diseases and conditions such as hypercholesterolemia and hypertriglyceridemia. Improvement of human cognition is also within the scope of the present invention (Webb T R et al., X-linked megalocornea caused by mutations in CHRDL1 identifies an essential role for ventroptin in anterior segment development. 2012. Am. J. Hum. Genet. 90:247-259). Inhibitors of CHRDL-1 and/or CHRDL-1 activity can be used for the prevention of neurodegeneration and/or to treat brain-related disorders and/or diseases (e.g., dementia; Alzheimer's disease; Parkinson's disease).

The present disclosure thus provides methods of treating or preventing a number of diseases related to undesirable CHRDL-1 levels and/or activity. The invention further provides antigen binding proteins, such as antibodies, that inhibit or modulate CHRDL-1 activity.

As used herein, the term “inhibiting an activity of CHRDL-1” refers to the ability of an antigen binding protein (such as an antibody) of the invention to inhibit or modulate one or more of the activities of CHRDL-1.

In one embodiment, the invention relates to methods of treating diabetes in a patient comprising administering to said patient an effective amount of an antigen binding protein capable of inhibiting an activity of CHRDL-1. In another embodiment, the invention relates to a method of treating a diabetes-related condition or disorder in a patient comprising administering to said patient an effective amount of an antigen binding protein capable of inhibiting an activity of CHRDL-1. The diabetes-related condition or disorder can be at least one of diabetic retinopathy or diabetic nephropathy.

In another embodiment, the invention relates to a method of treating obesity in a patient comprising administering to said patient an effective amount of an antigen binding protein capable of inhibiting an activity of CHRDL-1. In another embodiment, the invention relates to a method of modulating blood glucose in a patient comprising administering to said patient an effective amount of an antigen binding protein capable of inhibiting an activity of CHRDL-1.

The method further relates to a method of inducing and/or preserving brown fat formation and/or activity in a patient comprising administering to said patient an effective amount of an antigen binding protein capable of inhibiting an activity of CHRDL-1, as well as to a method of treating insulin resistance in a patient comprising administering to said patient an effective amount of an antigen binding protein capable of inhibiting an activity of CHRDL-1.

Additionally, another embodiment relates to a method of treating inflammatory disease in a patient comprising administering to said patient an effective amount of an antigen binding protein capable of inhibiting an activity of CHRDL-1.

In another embodiment, the invention relates to a method of treating dyslipidemia in a patient comprising administering to said patient an effective amount of an antigen binding protein capable of inhibiting an activity of CHRDL-1.

In still another embodiment, the invention relates to a method of treating a disease or disorder characterized by undesired levels of triglycerides in a patient comprising administering to said patient an effective amount of an antigen binding protein capable of inhibiting an activity of CHRDL-1.

In another embodiment, the invention relates to these methods using an antigen binding protein, where the antigen binding protein is an antibody comprising SEQ ID NO: 8, SEQ ID NO: 9, and SEQ ID NO: 10. In another embodiment, the invention relates to these methods wherein the antigen binding protein is an antibody comprising SEQ ID NO: 20, SEQ ID NO: 21, and SEQ ID NO: 22.

In another embodiment, the invention relates to these methods wherein said antigen binding protein is an antibody comprising SEQ ID NO: 26, SEQ ID NO: 27 and SEQ ID NO: 28.

In another embodiment, the invention relates to these methods wherein the isolated antigen binding protein comprises SEQ ID NO: 8, SEQ ID NO: 9, and SEQ ID NO: 10.

In another embodiment, the invention relates to these methods wherein said antigen binding protein is an antibody comprising SEQ ID NO: 37, SEQ ID NO: 38 and SEQ ID NO: 39.

In another embodiment, the invention relates to these methods wherein said antigen binding protein is an antibody comprising SEQ ID NO: 40, SEQ ID NO: 41 and SEQ ID NO: 42.

In yet another embodiment, the invention relates to an isolated antigen binding protein comprising SEQ ID NO: 20, SEQ ID NO: 21, and SEQ ID NO: 22. In yet another embodiment, the invention relates to an isolated antigen binding protein comprising SEQ ID NO: 26, SEQ ID NO: 27 and SEQ ID NO: 28.

In yet another embodiment, the invention relates to an isolated antigen binding protein comprising SEQ ID NO: 8, SEQ ID NO: 9, and SEQ ID NO: 10.

In yet another embodiment, the invention relates to an isolated antigen binding protein comprising SEQ ID NO: 37, SEQ ID NO: 38, and SEQ ID NO: 39. In yet another embodiment, the invention relates to an isolated antigen binding protein comprising SEQ ID NO: 40, SEQ ID NO: 41 and SEQ ID NO: 42.

The invention further relates to an antibody comprising SEQ ID NO: 49, an antibody comprising SEQ ID NO: 50, an antibody comprising SEQ ID NO: 55, an antibody comprising SEQ ID NO: 56, an antibody comprising SEQ ID NO: 57, an antibody comprising SEQ ID NO: 58, an antibody comprising SEQ ID NO: 59, an antibody comprising SEQ ID NO: 60, an antibody comprising SEQ ID NO: 61, an antibody comprising SEQ ID NO: 62, an antibody comprising SEQ ID NO: 65, an antibody comprising SEQ ID NO: 66, an antibody comprising SEQ ID NO: 69, an antibody comprising SEQ ID NO: 70, an antibody comprising SEQ ID NO: 71, and/or an antibody comprising SEQ ID NO: 72.

It will be appreciated that the antigen binding protein can be an antibody, such as a humanized antibody.

In another embodiment, the invention relates to an antibody that is cross-blocked by, or is capable of cross-blocking, an antigen binding protein as described herein.

In still another embodiment, the invention relates to a pharmaceutical composition comprising an antibody described herein.

In another embodiment, the invention relates to an isolated antigen binding protein having at least 85% sequence identity with any of SEQ ID NO: 8, 9, 10, 20, 21, 22, 26, 37, 38, 39, 40, 41, and 42.

The invention also relates to an isolated antigen binding protein having at least 90% sequence identity with any of SEQ ID NO: 8, 9, 10, 20, 21, 22, 26, 37, 38, 39, 40, 41, and 42.

In another embodiment, the invention relates to an isolated antigen binding protein having at least 95% sequence identity with any of SEQ ID NO: 8, 9, 10, 20, 21, 22, 26, 37, 38, 39, 40, 41, and 42.

In another embodiment, the invention relates to an isolated antigen binding protein comprising a variable region light chain having at least 85% sequence identity with any of light chain SEQ ID NOs: 29, 43, 47, 48, 63, and 64.

In still another embodiment, the invention relates to an isolated antigen binding protein comprising a variable region light chain having at least 90% sequence identity with any of light chain SEQ ID NOs: 29, 43, 47, 48, 63, and 64.

In yet another embodiment, the invention relates to an isolated antigen binding protein comprising a variable region light chain having at least 95% sequence identity with any of light chain SEQ ID NOs: 29, 43, 47, 48, 63, and 64.

In another embodiment, the invention relates to an isolated antigen binding protein comprising a variable region heavy chain having at least 85% sequence identity with any of light chain SEQ ID NOs: 31, 35, 45, 51, 52, 53, 54, 67, and 68.

In another embodiment, the invention relates to an isolated antigen binding protein comprising a variable region heavy chain having at least 90% sequence identity with any of light chain SEQ ID NOs: 31, 35, 45, 51, 52, 53, 54, 67, and 68.

In yet another embodiment, the invention relates to an isolated antigen binding protein comprising a variable region heavy chain having at least 95% sequence identity with any of light chain SEQ ID NOs: 31, 35, 45, 51, 52, 53, 54, 67, and 68.

In still another embodiment, the invention relates to an isolated antigen binding protein comprising a variable region light chain having at least 85% sequence identity with any of light chain SEQ ID NOs: 85, 93, 101, 109, 117, 125, 133, 141, and 149.

In another embodiment, the invention relates to an isolated antigen binding protein comprising a variable region light chain having at least 90% sequence identity with any of light chain SEQ ID NOs: 85, 93, 101, 109, 117, 125, 133, 141, and 149.

In another embodiment, the invention relates to an isolated antigen binding protein comprising a variable region light chain having at least 95% sequence identity with any of light chain SEQ ID NOs: 85, 93, 101, 109, 117, 125, 133, 141, and 149.

In another embodiment, the invention relates to an isolated antigen binding protein comprising a variable region heavy chain having at least 85% sequence identity with any of light chain SEQ ID NOs: 86, 94, 102, 110, 118, 126, 134, 142, and 150.

In another embodiment, the invention relates to an isolated antigen binding protein comprising a variable region heavy chain having at least 90% sequence identity with any of light chain SEQ ID NOs: 86, 94, 102, 110, 118, 126, 134, 142, and 150.

In another embodiment, the invention relates to an isolated antigen binding protein comprising a variable region heavy chain having at least 95% sequence identity with any of light chain SEQ ID NOs: 86, 94, 102, 110, 118, 126, 134, 142, and 150.

In another embodiment, the invention relates to an isolated antigen binding protein comprising SEQ ID NO: 79, SEQ ID NO: 80 and SEQ ID NO: 81. In another embodiment, the invention relates to an isolated antigen binding protein comprising SEQ ID NO: 82, SEQ ID NO: 83 and SEQ ID NO: 84. In another embodiment, the invention relates to an isolated antigen binding protein comprising SEQ ID NO: 87, SEQ ID NO: 88 and SEQ ID NO: 89. In another embodiment, the invention relates to an isolated antigen binding protein comprising SEQ ID NO: 90, SEQ ID NO: 91 and SEQ ID NO: 92. In another embodiment, the invention relates to an isolated antigen binding protein comprising SEQ ID NO: 95, SEQ ID NO: 96 and SEQ ID NO: 97. In another embodiment, the invention relates to an isolated antigen binding protein comprising SEQ ID NO: 98, SEQ ID NO: 99 and SEQ ID NO: 100. In another embodiment, the invention relates to an isolated antigen binding protein comprising SEQ ID NO: 103, SEQ ID NO: 104 and SEQ ID NO: 105. In another embodiment, the invention relates to an isolated antigen binding protein comprising SEQ ID NO: 106, SEQ ID NO: 107, and SEQ ID NO: 108. In another embodiment, the invention relates to an isolated antigen binding protein comprising SEQ ID NO: 111, SEQ ID NO: 112, and SEQ ID NO: 113. In another embodiment, the invention relates to an isolated antigen binding protein comprising SEQ ID NO: 114, SEQ ID NO: 115, and SEQ ID NO: 116. In another embodiment, the invention relates to an isolated antigen binding protein comprising SEQ ID NO: 119, SEQ ID NO: 120 and SEQ ID NO: 121. In another embodiment, the invention relates to an isolated antigen binding protein comprising SEQ ID NO: 122, SEQ ID NO: 123, and SEQ ID NO: 124. In another embodiment, the invention relates to an isolated antigen binding protein comprising SEQ ID NO: 127, SEQ ID NO: 128 and SEQ ID NO: 129. In another embodiment, the invention relates to an isolated antigen binding protein comprising SEQ ID NO: 130, SEQ ID NO: 131 and SEQ ID NO: 132. In another embodiment, the invention relates to an isolated antigen binding protein comprising SEQ ID NO: 135, SEQ ID NO: 136 and SEQ ID NO: 137. In another embodiment, the invention relates to an isolated antigen binding protein comprising SEQ ID NO: 138, SEQ ID NO: 139 and SEQ ID NO: 140. In another embodiment, the invention relates to an isolated antigen binding protein comprising SEQ ID NO: 143, SEQ ID NO: 144 and SEQ ID NO: 145. In another embodiment, the invention relates to an isolated antigen binding protein comprising SEQ ID NO: 146, SEQ ID NO: 147 and SEQ ID NO: 148.

It will be further appreciated that the invention relates to an isolated antigen binding protein that comprises at least one of the CDRs described herein, wherein the CDR comprises at least one of SEQ ID NO: 79, SEQ ID NO: 80, SEQ ID NO: 81, SEQ ID NO: 82, SEQ ID NO: 83, SEQ ID NO: 84, SEQ ID NO: 87, SEQ ID NO: 88, SEQ ID NO: 89, SEQ ID NO: 90, SEQ ID NO: 91, SEQ ID NO: 92, SEQ ID NO: 95, SEQ ID NO: 96, SEQ ID NO: 97, SEQ ID NO: 98, SEQ ID NO: 99, SEQ ID NO: 100, SEQ ID NO: 103, SEQ ID NO: 104, SEQ ID NO: 105, SEQ ID NO: 106, SEQ ID NO: 107, SEQ ID NO: 108, SEQ ID NO: 111, SEQ ID NO: 112, SEQ ID NO: 113, SEQ ID NO: 114, SEQ ID NO: 115, SEQ ID NO: 116, SEQ ID NO: 119, SEQ ID NO: 120, SEQ ID NO: 121, SEQ ID NO: 122, SEQ ID NO: 123, SEQ ID NO: 124, SEQ ID NO: 127, SEQ ID NO: 128, SEQ ID NO: 129, SEQ ID NO: 130, SEQ ID NO: 131, SEQ ID NO: 132, SEQ ID NO: 135, SEQ ID NO: 136, SEQ ID NO: 137, SEQ ID NO: 138, SEQ ID NO: 139, SEQ ID NO: 140, SEQ ID NO: 143, SEQ ID NO: 144, SEQ ID NO: 145, SEQ ID NO: 146, SEQ ID NO: 147, and SEQ ID NO: 148.

In yet another embodiment, the invention relates to a method of converting white adipose tissue to brown adipose tissue comprising administering to a patient an effective amount of a selective binding agent to CHRDL-1.

In another embodiment, the invention relates to a method of stimulating and/or promoting brown adipose tissue production (and/or preventing conversion of brown adipose tissue to white adipose tissue) comprising administering to a patient an effective amount of a selective binding agent to CHRDL-1.

The antigen binding proteins described herein can be a human antibody, a humanized antibody, chimeric antibody, a monoclonal antibody, a polyclonal antibody, a recombinant antibody, an antigen-binding antibody fragment, a single chain antibody, a diabody, a triabody, a tetrabody, a Fab fragment, an F(fab′)₂ fragment, a domain antibody, an IgD antibody, an IgE antibody, an IgM antibody, an IgG1 antibody, an IgG2 antibody, an IgG3 antibody, an IgG4 antibody, or an IgG4 antibody having at least one mutation in the hinge region.

The invention further relates to a nucleic acid comprising any of SEQ ID NOS: 5, 6, 7, 11, 12, 13, 17, 18, 19, 23, 24, 25, 30, 32, 34, 36, 44, 46, 73, 74, 75, 76, 77, and 78, expression vectors comprising one or more of these nucleic acids, as well as isolated cells comprising these expression vectors.

In yet another embodiment, the invention relates to a method of producing an antigen binding protein comprising incubating the cells comprising these expression vectors under conditions that allow it to express the antigen binding protein.

In still another embodiment, the invention relates to methods of preventing or treating a condition in a subject in need of such treatment comprising administering a therapeutically effective amount of the antigen binding proteins, such as antibodies, to the subject, wherein the condition is treatable by lowering one or more of blood glucose, insulin, or serum lipid levels. The condition can be, e.g., diabetes (such as type 2 diabetes), obesity, dyslipidemia, NASH, cardiovascular disease, or metabolic syndrome.

Other embodiments include methods of treating or preventing a disease, disorder, or condition in a subject in need of such treatment comprising administering a therapeutically effective amount of an antigen binding protein, such as an antibody, or pharmaceutical composition thereof to a subject, wherein the condition is treatable by lowering blood glucose, insulin or serum lipid levels. In certain embodiments, the disease, disorder or condition is, e.g., type 2 diabetes, obesity, dyslipidemia, NASH, cardiovascular disease or metabolic syndrome.

These and other aspects are described in greater detail herein. Each of the aspects provided can encompass various embodiments provided herein. It is therefore anticipated that each of the embodiments involving one element or combinations of elements can be included in each aspect described, and all such combinations of the above aspects and embodiments are expressly considered. Other features, objects, and advantages of the disclosed antigen binding proteins and associated methods and compositions are apparent in the detailed description that follows.

### BRIEF DESCRIPTION OF THE FIGURES

FIGS. 1A & 1B set forth the CDR sequences (amino acid and nucleic acid) of exemplary anti-CHRDL-1 antibodies TC1E3.1 (a.k.a., “1E3.1”, or “E3.1”) and TC2E1.1 (a.k.a., “2E1.1” or “E1.1”). Antibody TC1E3.1 is a mouse IgG1 HC and kappa LC antibody. Antibody TC2E1.1 is a mouse IgG1 HC and kappa LC antibody. FIG. 1A shows exemplary light chain CDR sequences, and FIG. 1B shows exemplary heavy chain CDR sequences. The term “LC” refers to the light chain, and “HC” refers to the heavy chain.

FIG. 2 sets forth the variable domain sequences (amino acid and nucleic acid) of anti-CHRDL-1 antibodies TC1E3.1 and TC2E1.1.

FIG. 3 shows the phenotype of CHRDL-1 knockout mice. Eight wild-type (WT) or knockout (KO) male mice were placed on a high fat diet (HFD) and body weights taken weekly followed by an intra-peritoneal glucose tolerance test (IP-GTT) at the end of the eight weeks. The collective percent weight gain and the IP-GTT are shown.

FIG. 4 shows brown adipose tissue (BAT) in CHRDL-1 knockout mice. BAT from wild-type (WT) or knockout (KO) was excised and fixed with 10% formalin. Tissue sections were cut and then stained with hematoxylin and eosin (H&E) or subjected to immunohistochemistry (IHC) using Uncoupled Protein 1 (Ucp1) antibodies. Cells positive for Ucp1 are stained brown.

FIG. 5 shows a cholesterol profile of CHRDL-1 knockout mice. Eight wild-type (WT) and knockout (KO) male mice were fasted for 12 hours and subsequently serum was drawn from all mice. Serum high density lipoprotein cholesterol (HDL-C) and low density lipoprotein cholesterol (LDL-C) were measured. An unparied T-test was used for statistical analysis.

FIG. 6 shows a cell-based assay to identify CHRDL-1 neutralizing antibodies. Mouse CHRDL-1 (left) and human CHRDL-1 (right) were used at 2.5 ng/ml. Monoclonal antibodies were used at 20 ng/ml. Relative Light Units from expression of the BRE-luciferase reporter gene are a relative measure of BMP signaling.

FIG. 7 shows humanized antibody sequences for the 1E3.1 and 2E1.1 antibodies, e.g., the CDRs within variable region and full length human IgG framework (including the constant region) with appropriate back-mutations. Constant region sequences are set forth in bold+italics, and the CDRs are underlined.

FIG. 8 shows the amino acid and nucleic acid sequences of the CDRs and variable regions of antibody 2G2.2 which is a mouse IgG1 HC and kappa LC antibody.

FIG. 9 shows an in vivo assay to identify antigen binding proteins to CHRDL-1 (e.g. 2G2.2) that improve metabolic-related parameters (e.g. weight, glucose tolerance). Percent body weight gain and glucose tolerance test (GTT) are shown for mice on high fat diet (HFD) treated concurrently with monoclonal antibodies (mAbs). Groups of mice were treated with vehicle (N=10), 2G2.2 (N=8), or 1E3.1 (N=10) mAbs once weekly for ten weeks while on HFD. The upper graph represents the percent body weight gain while on HFD while the lower graph represents GTTs following ten weeks on HFD.

FIG. 10 shows serum parameters in mice on high fat diet (HFD) treated concurrently with monoclonal antibodies (mAbs). Serum from mice treated with vehicle (N=8), 2G2.2 (N=8), or 1E3.1 (N=9) mAbs once weekly for ten weeks while on HFD was collected and analyzed for abundance of several serum proteins.

FIG. 11 shows lipid serum parameters in mice on high fat diet (HFD) treated concurrently with monoclonal antibodies (mAbs). Serum from mice treated with vehicle (N=9), 2G2.2 (N=8), or 1E3.1 (N=9) mAbs once weekly for ten weeks while on HFD was collected and analyzed for total cholesterol, low-density lipoproteins (LDL-C), high density lipoproteins (HDL-C), or triglycerides.

FIG. 12 shows humanized antibody sequences for the 2G2.2 antibody, e.g., the CDRs within variable region and full length human IgG framework (including the constant region) with appropriate back-mutations. Constant region sequences are set forth in bold+italics, and the CDRs are underlined. The humanized antibody having light chain huz-LC2G2.2_LC (SEQ ID NO: 65) and heavy chain huz-bmHC2G2.2_eflsIgG1 (SEQ ID NO: 72) is referred to herein as “huz-LC2G2.2_LC/huz-bmHC2G2.2_eflsIgG1” or “huz-2G2.2-A.”

FIG. 13 shows the nucleic acid sequences of the CDRs of antibody 2G2.2 which is a mouse IgG1 HC and kappa LC antibody.

FIG. 14 shows that CHRDL-1 inhibits brown fat formation as shown by microarray analysis of brown and white adipose tissue from wild-type and CHRLD-1 knock-out mice. The three columns on the left present data listing the most highly changed genes in wild-type brown adipose when compared to wild-type white adipose. The three columns on the right present data listing the most highly changed genes in CHRDL-1 knock-out white adipose when compared to wild-type white adipose.

FIG. 15 shows the effects of CHRDL-1 monoclonal antibodies on weight gain and glucose tolerance in mice with established obesity. Mice were fed a high fat diet for six weeks and then treated with monoclonal antibodies. Groups of mice were treated with vehicle (N=10), 2G2.2 (N=8), or 2E1.1 (N=10) monoclonal antibodies once weekly for six weeks while on high fat diet. The top graph represents the percent body weight gain while on high fat diet. The bottom graph represents glucose tolerance tests while on high fat diet.

FIG. 16 shows that humanized 2G2 reduces weight gain in mice with established obesity. Mice were fed a high fat diet for six weeks and then treated with monoclonal antibodies. Groups of mice were treated with vehicle (N=11), 2G2.2 (mu2G2.2, N=12), humanized 2G2 (Huz-2G2.2-A, N=12) or 1H6.2 (N=11) mAbs once weekly for seven weeks while on HFD. The graph represents the total body weight gain in grams while on the high fat diet.

FIG. 17 shows the amino acid sequences of the CDRs, light chain and heavy chain of mouse antibody 1H6.2. Constant region sequences are set forth in bold+italics, and the CDRs are underlined.

FIG. 18 shows the amino acid sequences of the CDRs, light chain and heavy chain of mouse antibody TC3.2.1. Constant region sequences are set forth in bold+italics, and the CDRs are underlined.

FIG. 19 shows the amino acid sequences of the CDRs, light chain and heavy chain of mouse antibody 3B9.1. Constant region sequences are set forth in bold+italics, and the CDRs are underlined.

FIG. 20 shows the amino acid sequences of the CDRs, light chain and heavy chain of mouse antibody 3C11.2. Constant region sequences are set forth in bold+italics, and the CDRs are underlined.

FIG. 21 shows the amino acid sequences of the CDRs, light chain and heavy chain of mouse antibody 1A11.2. Constant region sequences are set forth in bold+italics, and the CDRs are underlined.

FIG. 22 shows the amino acid sequences of the CDRs, light chain and heavy chain of mouse antibody 1G12.1. Constant region sequences are set forth in bold+italics, and the CDRs are underlined.

FIG. 23 shows the amino acid sequences of the CDRs, light chain and heavy chain of mouse antibody 3B2.1. Constant region sequences are set forth in bold+italics, and the CDRs are underlined.

FIG. 24 shows the amino acid sequences of the CDRs, light chain and heavy chain of mouse antibody 3G4.1. Constant region sequences are set forth in bold+italics, and the CDRs are underlined.

FIG. 25 shows the amino acid sequences of the CDRs, light chain and heavy chain of mouse antibody 3H6.2. Constant region sequences are set forth in bold+italics, and the CDRs are underlined.

FIG. 26 shows the amino acid sequences of the CDRs and variable regions of antibody 16E6.1 which is a Xenomouse® fully human antibody with a kappa LC and either a IgG2 or a IgG4 HC. The CDRs are underlined.

FIG. 27 shows brown adipose tissue (BAT) in 2G2.2 treated mice. BAT from 2G2.2 and vehicle treated mice on high fat diet was excised and fixed with 10% formalin. Tissue sections were cut and then stained with hematoxylin and eosin (H&E).

### DETAILED DESCRIPTION

The section headings used herein are for organizational purposes only and are not to be construed as limiting the subject matter described.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present application are generally performed according to conventional methods well known in the art and as described in various general and more specific references that are cited and discussed throughout the present specification unless otherwise indicated. See e.g., Sambrook et al., Molecular Cloning: A Laboratory Manual, 3^{rd} ed., Cold Spring Harbor Laboratory Press, Cold Spring Harbor, N.Y. (2001) and subsequent editions, Ausubel et al., Current Protocols in Molecular Biology, Greene Publishing Associates (1992), and Harlow & Lane, Antibodies: A Laboratory Manual, Cold Spring Harbor Laboratory Press, Cold Spring Harbor, N.Y. (1988), which are incorporated herein by reference. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients.

It should be understood that the instant disclosure is not limited to the particular methodology, protocols, and reagents, etc., described herein and as such can vary. The terminology used herein is for the purpose of describing particular embodiments only, and is not intended to limit the scope of the present disclosure.

Other than in the operating examples, or where otherwise indicated, all numbers expressing quantities of ingredients or reaction conditions used herein should be understood as modified in all instances by the term “about.” The term “about” when used in connection with percentages can mean ±5%, e.g., 1%, 2%, 3%, or 4%.

### DEFINITIONS

As used herein, the terms “a” and “an” mean “one or more” unless specifically stated otherwise.

As used herein, an “antigen binding protein” is a protein comprising a portion that binds to an antigen or target and, optionally, a scaffold or framework portion that allows the antigen binding portion to adopt a conformation that promotes binding of the antigen binding protein to the antigen. Examples of antigen binding proteins antibodies such as a human antibody, a humanized antibody; a chimeric antibody; a recombinant antibody; a single chain antibody; a diabody; a triabody; a tetrabody; a Fab fragment; a F(ab′)₂ fragment; an IgD antibody; an IgE antibody; an IgM antibody; an IgG1 antibody; an IgG2 antibody; an IgG3 antibody; or an IgG4 antibody, and fragments thereof. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See e.g., Korndorfer et al., (2003) Proteins: Structure, Function, and Bioinformatics, 53(1):121-129; Roque et al., (2004) Biotechnol. Prog. 20:639-654. In addition, peptide antibody mimetics (“PAMs”) can be used, as well as scaffolds based on antibody mimetics utilizing fibronectin components as a scaffold.

An antigen binding protein can have, for example, the structure of a naturally occurring immunoglobulin. An “immunoglobulin” is a tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology 2^{nd} Ed., Ch. 7 (Paul, W., ed., Raven Press, N.Y. (1989)), incorporated by reference herein in its entirety. The variable regions of each light/heavy chain pair form the antibody binding site such that an intact immunoglobulin has two binding sites.

Naturally occurring immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3, and FR4. The assignment of amino acids to each domain can be done in accordance with the definitions of Kabat et al., (1991) “Sequences of Proteins of Immunological Interest”, 5^{th} Ed., U.S. Dept. of Health & Human Services, PHS, NIH, NIH Publication no. 91-3242. Although presented herein using the Kabat nomenclature system, as desired, the CDRs disclosed herein can also be redefined according an alternative nomenclature scheme, such as that of Chothia (see Chothia & Lesk, (1987) J. Mol. Biol. 196:901-917; Chothia et al., (1989) Nature 342:878-883 or Honegger & Pluckthun, (2001) J. Mol. Biol. 309:657-670).

In the context of the instant disclosure an antigen binding protein is said to “specifically bind” or “selectively bind” its target antigen when the dissociation constant (K_{D}) is ≦10⁻⁸ M. The antibody specifically binds antigen with “high affinity” when the K_{D} is ≦5×10⁻⁹ M, and with “very high affinity” when the K_{D} is ≦5×10⁻¹⁰ M. In one embodiment, the antibodies will bind to CHRDL-1 with a K_{D} of between about 10⁻⁷ M and 10⁻¹² M, and in yet another embodiment the antibodies will bind with a K_{D}≦5×10⁻⁹.

An “antibody” refers to an intact immunoglobulin or to an antigen binding portion thereof that competes with the intact antibody for specific binding, unless otherwise specified. Antigen binding portions can be produced by recombinant DNA techniques or by enzymatic or chemical cleavage of intact antibodies. Antigen binding portions include, inter alia, Fab, Fab′, F(ab′)₂, Fv, domain antibodies (dAbs), fragments including complementarity determining regions (CDRs), single-chain antibodies (scFv), chimeric antibodies, diabodies, triabodies, tetrabodies, and polypeptides that contain at least a portion of an immunoglobulin that is sufficient to confer specific antigen binding to the polypeptide.

A Fab fragment is a monovalent fragment having the V_{L}, V_{H}, C_{L} and C_{H}1 domains; a F(ab′)₂ fragment is a bivalent fragment having two Fab fragments linked by a disulfide bridge at the hinge region; a Fd fragment has the V_{H} and C_{H}1 domains; an Fv fragment has the V_{L} and V_{H} domains of a single arm of an antibody; and a dAb fragment has a V_{H} domain, a V_{L} domain, or an antigen-binding fragment of a V_{H} or V_{L} domain (U.S. Pat. Nos. 6,846,634, and 6,696,245; and U.S. application publications numbers 05/0202512, 04/0202995, 04/0038291, 04/0009507, 03/0039958, Ward et al., Nature 341:544-546 (1989)).

A single-chain antibody (scFv) is an antibody in which a V_{L} and a V_{H} region are joined via a linker (e.g., a synthetic sequence of amino acid residues) to form a continuous protein chain wherein the linker is long enough to allow the protein chain to fold back on itself and form a monovalent antigen binding site (See e.g., Bird et al., (1988) Science 242:423-26 and Huston et al., (1988) Proc. Natl. Acad. Sci. USA 85:5879-83). Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (See e.g., Holliger et al., (1993) Proc. Natl. Acad. Sci. USA 90:6444-48, and Poljak et al., (1994) Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

Complementarity determining regions (CDRs) and framework regions (FR) of a given antibody can be identified using the system described by Kabat et al., (1991) “Sequences of Proteins of Immunological Interest”, 5^{th} Ed., U.S. Dept. of Health and Human Services, PHS, NIH, NIH Publication no. 91-3242. Although presented using the Kabat nomenclature system, as desired, the CDRs disclosed herein can also be redefined according an alternative nomenclature scheme, such as that of Chothia (see Chothia & Lesk, (1987) J. Mol. Biol. 196:901-917; Chothia et al., (1989) Nature 342:878-883 or Honegger & Pluckthun, (2001) J. Mol. Biol. 309:657-670). One or more CDRs can be incorporated into a molecule either covalently or noncovalently to make it an antigen binding protein. An antigen binding protein can incorporate the CDR(s) as part of a larger polypeptide chain, can covalently link the CDR(s) to another polypeptide chain, or can incorporate the CDR(s) noncovalently. The CDRs permit the antigen binding protein to specifically bind to a particular antigen of interest.

An antigen binding protein can but need not have one or more binding sites. If there is more than one binding site, the binding sites can be identical to one another or can be different. For example, a naturally occurring human immunoglobulin typically has two identical binding sites, while a “bispecific” or “bifunctional” antibody has two different binding sites. Antigen binding proteins of this bispecific form (e.g., those comprising various heavy and light chain CDRs provided herein) comprise aspects of the instant disclosure.

The term “human antibody” includes all antibodies that have one or more variable and constant regions derived from human immunoglobulin sequences. In one embodiment, all of the variable and constant domains are derived from human immunoglobulin sequences (a fully human antibody). These antibodies can be prepared in a variety of ways, examples of which are described below, including through the immunization with an antigen of interest of a mouse that is genetically modified to express antibodies derived from human heavy and/or light chain-encoding genes, such as a mouse derived from a Xenomouse®, UltiMab®, HuMAb-Mouse®, Velocimouse®, Velocimmune®, KyMouse™, or AlivaMab™ system, or derived from human heavy chain transgenic mouse, transgenic rat human antibody repertoire, transgenic rabbit human antibody repertoire or cow human antibody repertoire or HuTarg™ technology. Phage-based approaches can also be employed.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject.

In one embodiment, one or more of the CDRs are derived from a murine antibody that binds to CHRDL-1. In another embodiment, all of the CDRs are derived from a murine antibody that binds to CHRDL-1. In another embodiment, the CDRs from more than one murine antibody that binds to CHRDL-1 can be used. It will be further appreciated that the framework regions can be derived from one or more of the same antibodies that bind to CHRDL-1, such as a human antibody, or from a humanized antibody, or the like. Thus, in some embodiments, certain amino acids in the framework and constant domains of the heavy and/or light chains of the antibody are mutated to produce the humanized antibody. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies can be found in U.S. Pat. Nos. 6,054,297, 5,886,152, and 5,877,293.

The term “chimeric antibody” refers to an antibody that contains one or more regions from one antibody and one or more regions from one or more other antibodies. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody or antibodies from another species or belonging to another antibody class or subclass. Also included are fragments of such antibodies that exhibit the desired biological activity (e.g., the ability to specifically bind to CHRDL-1).

The term “light chain” includes a full-length light chain and fragments thereof having sufficient variable region sequence to confer binding specificity. A full-length light chain includes a variable region domain, V_{L}, and a constant region domain, C_{L}. The variable region domain of the light chain is at the amino-terminus of the polypeptide. Light chains include kappa (“κ”) chains and lambda (“λ”) chains.

The term “heavy chain” includes a full-length heavy chain and fragments thereof having sufficient variable region sequence to confer binding specificity. A full-length heavy chain includes a variable region domain, V_{H}, and three constant region domains, C_{H}1, C_{H}2, and C_{H}3. The V_{H} domain is at the amino-terminus of the polypeptide, and the C_{H} domains are at the carboxyl-terminus, with the C_{H}3 being closest to the carboxy-terminus of the polypeptide. Heavy chains can be of any isotype, including IgG (including IgG1, IgG2, IgG3 and IgG4 subtypes), IgA (including IgA1 and IgA2 subtypes), IgM and IgE.

The term “immunologically functional fragment” (or simply “fragment”) of an antigen binding protein, e.g., an antibody or immunoglobulin chain (heavy or light chain), as used herein, is an antigen binding protein comprising a portion (regardless of how that portion is obtained or synthesized) of an antibody that lacks at least some of the amino acids present in a full-length chain but which is capable of specifically binding to an antigen. Such fragments are biologically active in that they bind specifically to the target antigen and can compete with other antigen binding proteins, including intact antibodies, for specific binding to a given epitope. In one aspect, such a fragment will retain at least one CDR present in the full-length light or heavy chain, and in some embodiments will comprise a single heavy chain and/or light chain or portion thereof. These biologically active fragments can be produced by recombinant DNA techniques, or can be produced by enzymatic or chemical cleavage of antigen binding proteins, including intact antibodies. Immunologically functional immunoglobulin fragments include, but are not limited to, Fab, Fab′, F(ab′)₂, Fv, domain antibodies and single-chain antibodies, and can be derived from any mammalian source, including but not limited to human, mouse, rat, camelid or rabbit. It is contemplated further that a functional portion of the antigen binding proteins disclosed herein, for example, one or more CDRs, could be covalently bound to a second protein or to a small molecule to create a therapeutic agent directed to a particular target in the body, possessing bifunctional therapeutic properties, or having a prolonged serum half-life.

An “Fc” region contains two heavy chain fragments comprising the C_{H}2 and C_{H}3 domains of an antibody. The two heavy chain fragments are held together by two or more disulfide bonds and by hydrophobic interactions of the C_{H}3 domains.

An “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form an F(ab′)₂ molecule.

An “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody can target the same or different antigens.

A “hemibody” is an immunologically-functional immunoglobulin construct comprising a complete heavy chain, a complete light chain and a second heavy chain Fc region paired with the Fc region of the complete heavy chain. A linker can, but need not, be employed to join the heavy chain Fc region and the second heavy chain Fc region. In particular embodiments a hemibody is a monovalent form of an antigen binding protein disclosed herein. In other embodiments, pairs of charged residues can be employed to associate one Fc region with the second Fc region.

A “bivalent antigen binding protein” or “bivalent antibody” comprises two antigen binding sites. In some instances, the two binding sites have the same antigen specificities. Bivalent antigen binding proteins and bivalent antibodies can be bispecific, as described herein, and form aspects of the instant disclosure.

A “multispecific antigen binding protein” or “multispecific antibody” is one that targets more than one antigen or epitope, and forms another aspect of the instant disclosure.

A “bispecific,” “dual-specific”, or “bifunctional” antigen binding protein or antibody is a hybrid antigen binding protein or antibody, respectively, having two different antigen binding sites. Bispecific antigen binding proteins and antibodies are a species of multispecific antigen binding protein or multispecific antibody and can be produced by a variety of methods including, but not limited to, fusion of hybridomas or linking of Fab′ fragments. See e.g., Songsivilai & Lachmann, (1990) Clin. Exp. Immunol. 79:315-321; Kostelny et al., (1992) J. Immunol. 148:1547-1553. The two binding sites of a bispecific antigen binding protein or antibody will bind to two different epitopes, which can reside on the same or different protein targets.

The terms “inhibits CHRDL-1 activity,” and “modulates CHRDL-1 activity,” herein mean that the antigen binding proteins inhibit, or modulate, a biological effect induced by CHRDL-1. This may include CHRDL-1 signaling effects.

Antibodies according to the invention may have a binding affinity for human CHRDL-1 of less than or equal to 1×10⁻⁷M, less than or equal to 1×10⁻⁸M, less than or equal to 1×10⁻⁹M, less than or equal to 1×10⁻¹⁰M, less than or equal to 1×10⁻¹¹M, or less than or equal to 1×10⁻¹²M.

The affinity of a binding agent such as an antibody or binding partner, as well as the extent to which a binding agent (such as an antibody) inhibits binding, can be determined by one of ordinary skill in the art using conventional techniques, for example those described by Scatchard et al. (Ann. N.Y. Acad. Sci. 51:660-672 (1949)) or by KinExA® (Sapidyne Instruments, Inc., Boise, Id.) or by surface plasmon resonance (SPR; BIAcore®, Biosensor, Piscataway, N.J.). For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (See e.g., Wolff et al., Cancer Res. 53:2560-65 (1993)).

The term “polynucleotide” or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

The term “oligonucleotide” means a polynucleotide comprising 200 or fewer nucleotides. In some embodiments, oligonucleotides are 10 to 60 bases in length. In other embodiments, oligonucleotides are 12, 13, 14, 15, 16, 17, 18, 19, or 20-40 nucleotides in length. Oligonucleotides can be single stranded or double stranded, e.g., for use in the construction of a mutant gene. Oligonucleotides can be sense or antisense oligonucleotides. An oligonucleotide can include a label, including a radiolabel, a fluorescent label, a hapten or an antigenic label, for detection assays. Oligonucleotides can be used, for example, as PCR primers, cloning primers or hybridization probes.

An “isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it is understood that “a nucleic acid molecule comprising” a particular nucleotide sequence does not encompass intact chromosomes. Isolated nucleic acid molecules “comprising” specified nucleic acid sequences can include, in addition to the specified sequences, coding sequences for up to ten or even up to twenty other proteins or portions thereof, or can include operably linked regulatory sequences that control expression of the coding region of the recited nucleic acid sequences, and/or can include vector sequences.

Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

The term “control sequence” refers to a polynucleotide sequence that can affect the expression and processing of coding sequences to which it is ligated. The nature of such control sequences can depend upon the host organism. In particular embodiments, control sequences for prokaryotes can include a promoter, a ribosomal binding site, and a transcription termination sequence. For example, control sequences for eukaryotes can include promoters comprising one or a plurality of recognition sites for transcription factors, transcription enhancer sequences, and transcription termination sequence. “Control sequences” can include leader sequences and/or fusion partner sequences.

The term “vector” means any molecule or entity (e.g., nucleic acid, plasmid, bacteriophage or virus) used to transfer protein coding information into a host cell.

The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct can include, but is not limited to, sequences that affect or control transcription, translation, and, if introns are present, affect RNA splicing of a coding region operably linked thereto.

As used herein, “operably linked” means that the components to which the term is applied are in a relationship that allows them to carry out their inherent functions under suitable conditions. For example, a control sequence in a vector that is “operably linked” to a protein coding sequence is ligated thereto so that expression of the protein coding sequence is achieved under conditions compatible with the transcriptional activity of the control sequences.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

The term “transduction” means the transfer of genes from one bacterium to another, usually by bacteriophage. “Transduction” also refers to the acquisition and transfer of eukaryotic cellular sequences by replication-defective retroviruses.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See e.g., Graham et al., (1973) Virology 52:456; Sambrook et al., (2001), supra; Davis et al., (1986) Basic Methods in Molecular Biology, Elsevier; Chu et al., (1981) Gene 13:197. Such techniques can be used to introduce one or more exogenous DNA moieties into suitable host cells.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA can recombine with that of the cell by physically integrating into a chromosome of the cell, or can be maintained transiently as an episomal element without being replicated, or can replicate independently as a plasmid. A cell is considered to have been “stably transformed” when the transforming DNA is replicated with the division of the cell.

The terms “polypeptide” or “protein” are used interchangeably herein to refer to a polymer of amino acid residues. The terms also apply to amino acid polymers in which one or more amino acid residues is an analog or mimetic of a corresponding naturally occurring amino acid, as well as to naturally occurring amino acid polymers. The terms can also encompass amino acid polymers that have been modified, e.g., by the addition of carbohydrate residues to form glycoproteins, or phosphorylated. Polypeptides and proteins can be produced by a naturally-occurring and non-recombinant cell, or polypeptides and proteins can be produced by a genetically-engineered or recombinant cell. Polypeptides and proteins can comprise molecules having the amino acid sequence of a native protein, or molecules having deletions from, additions to, and/or substitutions of one or more amino acids of the native sequence. The terms “polypeptide” and “protein” encompass antigen binding proteins that specifically or selectively bind to CHRDL-1 or sequences that have deletions from, additions to, and/or substitutions of one or more amino acids of an antigen binding protein that specifically or selectively binds to CHRDL-1. The term “polypeptide fragment” refers to a polypeptide that has an amino-terminal deletion, a carboxyl-terminal deletion, and/or an internal deletion as compared with the full-length protein. Such fragments can also contain modified amino acids as compared with the full-length protein. In certain embodiments, fragments are about five to 500 amino acids long. For example, fragments can be at least 5, 6, 8, 10, 14, 20, 50, 70, 100, 110, 150, 200, 250, 300, 350, 400, or 450 amino acids long. Useful polypeptide fragments include immunologically functional fragments of antibodies, including binding domains. In the case of an antigen binding protein that binds to CHRDL-1, useful fragments include but are not limited to a CDR region, a variable domain of a heavy or light chain, a portion of an antibody chain or just its variable region including two CDRs, and the like.

The term “isolated protein” referred means that a subject protein (1) is free of at least some other proteins with which it would normally be found, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is operably associated (by covalent or noncovalent interaction) with a polypeptide with which it is not associated in nature, or (6) does not occur in nature. Typically, an “isolated protein” constitutes at least about 5%, at least about 10%, at least about 25%, or at least about 50% of a given sample. Genomic DNA, cDNA, mRNA or other RNA, of synthetic origin, or any combination thereof can encode such an isolated protein. Preferably, the isolated protein is substantially free from proteins or polypeptides or other contaminants that are found in its natural environment that would interfere with its therapeutic, diagnostic, prophylactic, research or other use.

A “variant” of a polypeptide (e.g., an antigen binding protein, or an antibody) comprises an amino acid sequence wherein one or more amino acid residues are inserted into, deleted from and/or substituted into the amino acid sequence relative to another polypeptide sequence. Variants include fusion proteins.

A “derivative” of a polypeptide is a polypeptide (e.g., an antigen binding protein, or an antibody) that has been chemically modified in some manner distinct from insertion, deletion, or substitution variants, e.g., by conjugation to another chemical moiety.

The term “naturally occurring” as used throughout the specification in connection with biological materials such as polypeptides, nucleic acids, host cells, and the like, refers to materials which are found in nature.

“Antigen binding region” means a protein, or a portion of a protein, that specifically binds a specified antigen, e.g., CHRDL-1. For example, the portion of an antigen binding protein that contains the amino acid residues that interact with an antigen and confer on the antigen binding protein its specificity and affinity for the antigen is referred to as “antigen binding region.” An antigen binding region typically includes one or more “complementary binding regions” (“CDRs”). Certain antigen binding regions also include one or more “framework” regions. A “CDR” is an amino acid sequence that contributes to antigen binding specificity and affinity. “Framework” regions can aid in maintaining the proper conformation of the CDRs to promote binding between the antigen binding region and an antigen.

In certain aspects, recombinant antigen binding proteins that bind to CHRDL-1 are provided. In this context, a “recombinant protein” is a protein made using recombinant techniques, i.e., through the expression of a recombinant nucleic acid as described herein. Methods and techniques for the production of recombinant proteins are well known in the art.

The term “compete” when used in the context of antigen binding proteins (e.g., neutralizing antigen binding proteins, neutralizing antibodies, antagonistic antigen binding proteins, agonistic antibodies, and binding proteins, are those that bind to a CHRDL-1 and compete for the same epitope or binding site on a target as the antigen binding proteins recited herein. This can be determined by various means in the art, including assays in which the antigen binding protein (e.g., antibody or immunologically functional fragment thereof) under study prevents or inhibits the specific binding of a reference molecule (e.g., a reference ligand, or reference antigen binding protein, such as a reference antibody) to a common antigen (e.g., CHRDL-1, or a fragment thereof, or a complex comprising CHRDL-1 and its receptor or receptors or binding partner/s).

Numerous types of competitive binding assays can be used to determine if a test molecule competes with a reference molecule for binding. Examples of assays that can be employed include solid phase direct or indirect radioimmunoassay (RIA), solid phase direct or indirect enzyme immunoassay (EIA), sandwich competition assay (See e.g., Stahli et al., (1983) Methods in Enzymology 9:242-253); solid phase direct biotin-avidin EIA (See e.g., Kirkland et al., (1986) J. Immunol. 137:3614-3619) solid phase direct labeled assay, solid phase direct labeled sandwich assay (See e.g., Harlow and Lane, (1988) supra); solid phase direct label RIA using 1-125 label (See e.g., Morel et al., (1988) Molec. Immunol. 25:7-15); solid phase direct biotin-avidin EIA (See e.g., Cheung, et al., (1990) Virology 176:546-552); and direct labeled RIA (Moldenhauer et al., (1990) Scand. J. Immunol. 32:77-82). Typically, such an assay involves the use of a purified antigen bound to a solid surface or cells bearing either of an unlabeled test antigen binding protein or a labeled reference antigen binding protein. Competitive inhibition is measured by determining the amount of label bound to the solid surface or cells in the presence of the test antigen binding protein. Usually the test antigen binding protein is present in excess. Antigen binding proteins identified by competition assay (competing antigen binding proteins) include antigen binding proteins binding to the same epitope as the reference antigen binding proteins and antigen binding proteins binding to an adjacent epitope sufficiently proximal to the epitope bound by the reference antigen binding protein for steric hindrance to occur. Additional details regarding methods for determining competitive binding are provided in the examples herein. Usually, when a competing antigen binding protein is present in excess, it will inhibit specific binding of a reference antigen binding protein to a common antigen by at least 70% or 75%. In some instance, binding is inhibited by at least 80%, 85%, 90%, 95%, or 97% or more.

The term “antigen” refers to a molecule or a portion of a molecule capable of being bound by a selective binding agent, such as an antigen binding protein (including, e.g., an antibody or immunological functional fragment thereof), and may also be capable of being used in an animal to produce antibodies capable of binding to that antigen. An antigen can possess one or more epitopes that are capable of interacting with different antigen binding proteins, e.g., antibodies.

The term “epitope” means the amino acids of a target molecule that are contacted by an antigen binding protein (for example, an antibody) when the antigen binding protein is bound to the target molecule. The term includes any subset of the complete list of amino acids of the target molecule that are contacted when an antigen binding protein, such as an antibody, is bound to the target molecule. An epitope can be contiguous or non-contiguous (e.g., (i) in a single-chain polypeptide, amino acid residues that are not contiguous to one another in the polypeptide sequence but that within in context of the target molecule are bound by the antigen binding protein, or (ii) in a multimeric receptor comprising two or more individual components, amino acid residues that are present on one or more of the individual components, but which are still bound by the antigen binding protein). In certain embodiments, epitopes can be mimetic in that they comprise a three dimensional structure that is similar to an antigenic epitope used to generate the antigen binding protein, yet comprise none or only some of the amino acid residues found in that epitope used to generate the antigen binding protein. Most often, epitopes reside on proteins, but in some instances can reside on other kinds of molecules, such as nucleic acids. Epitope determinants can include chemically active surface groupings of molecules such as amino acids, sugar side chains, phosphoryl or sulfonyl groups, and can have specific three dimensional structural characteristics, and/or specific charge characteristics. Generally, antigen binding proteins specific for a particular target molecule will preferentially recognize an epitope on the target molecule in a complex mixture of proteins and/or macromolecules.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), (1988) New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., (1987) Sequence Analysis in Molecular Biology, New York: Academic Press; Sequence Analysis Primer, (Gribskov, M. and Devereux, J., eds.), 1991, New York: M. Stockton Press; and Carillo et al., (1988) J. Applied Math. 48:1073.

In calculating percent identity, the sequences being compared are aligned in a way that gives the largest match between the sequences. The computer program used to determine percent identity is the GCG program package, which includes GAP (Devereux et al., (1984) Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM250 or BLOSUM62 are used in conjunction with the algorithm. In certain embodiments, a standard comparison matrix (see, Dayhoff et al., (1978) Atlas of Protein Sequence and Structure 5:345-352 for the PAM250 comparison matrix; Henikoff et al., (1992) Proc. Natl. Acad. Sci. U.S.A. 89:10915-10919 for the BLOSUM62 comparison matrix) is also used by the algorithm.

Recommended parameters for determining percent identity for polypeptides or nucleotide sequences using the GAP program are the following:
Algorithm: Needleman et al., 1970, J. Mol. Biol. 48:443-453;
Comparison matrix: BLOSUM 62 from Henikoff et al., 1992, supra;
Gap Penalty: 12 (but with no penalty for end gaps)
Gap Length Penalty: 4
Threshold of Similarity: 0

Certain alignment schemes for aligning two amino acid sequences can result in matching of only a short region of the two sequences, and this small aligned region can have very high sequence identity even though there is no significant relationship between the two full-length sequences. Accordingly, the selected alignment method (e.g., the GAP program) can be adjusted if so desired to result in an alignment that spans at least 50 contiguous amino acids of the target polypeptide.

As used herein, “substantially pure” means that the described species of molecule is the predominant species present, that is, on a molar basis it is more abundant than any other individual species in the same mixture. In certain embodiments, a substantially pure molecule is a composition wherein the object species comprises at least 50% (on a molar basis) of all macromolecular species present. In other embodiments, a substantially pure composition will comprise at least 80%, 85%, 90%, 95%, or 99% of all macromolecular species present in the composition. In other embodiments, the object species is purified to essential homogeneity wherein contaminating species cannot be detected in the composition by conventional detection methods and thus the composition consists of a single detectable macromolecular species.

The terms “treat” and “treating” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods presented herein can be employed to treat diabetes, (such as Type 2 diabetes), obesity and/or dyslipidemia, either prophylactically or as an acute treatment, to decrease plasma glucose levels, to decrease circulating triglyceride levels, to decrease circulating cholesterol levels and/or ameliorate a symptom associated with diabetes, obesity and dyslipidemia.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with diabetes, obesity and dyslipidemia. In some embodiments, the effective amount is a therapeutically effective amount or a prophylactically effective amount. A “therapeutically effective amount” is an amount sufficient to remedy a disease state (e.g., diabetes, obesity or dyslipidemia) or symptoms, particularly a state or symptoms associated with the disease state, or otherwise prevent, hinder, retard or reverse the progression of the disease state or any other undesirable symptom associated with the disease in any way whatsoever. A “prophylactically effective amount” is an amount of a pharmaceutical composition that, when administered to a subject, will have the intended prophylactic effect, e.g., preventing or delaying the onset (or reoccurrence) of diabetes, obesity or dyslipidemia, or reducing the likelihood of the onset (or reoccurrence) of diabetes, obesity or dyslipidemia or associated symptoms. The full therapeutic or prophylactic effect does not necessarily occur by administration of one dose, and may occur only after administration of a series of doses. Thus, a therapeutically or prophylactically effective amount can be administered in one or more administrations.

“Amino acid” takes its normal meaning in the art. The twenty naturally-occurring amino acids and their abbreviations follow conventional usage. See, Immunology—A Synthesis, 2^{nd} Edition, (E. S. Golub and D. R. Green, eds.), Sinauer Associates: Sunderland, Mass. (1991), incorporated herein by reference for any purpose. Stereoisomers (e.g., D-amino acids) of the twenty conventional amino acids, unnatural or non-naturally occurring or encoded amino acids such as α-,α-disubstituted amino acids, N-alkyl amino acids, and other unconventional amino acids can also be suitable components for polypeptides and are included in the phrase “amino acid.” Examples of non-natural and non-naturally encoded amino acids (which can be substituted for any naturally-occurring amino acid found in any sequence disclosed herein, as desired) include: 4-hydroxyproline, γ-carboxyglutamate, ε-N,N,N-trimethyllysine, E-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention. A non-limiting lists of examples of non-naturally occurring/encoded amino acids that can be inserted into an antigen binding protein sequence or substituted for a wild-type residue in an antigen binding sequence include β-amino acids, homoamino acids, cyclic amino acids and amino acids with derivatized side chains. Examples include (in the L-form or D-form; abbreviated as in parentheses): citrulline (Cit), homocitrulline (hCit), Nα-methylcitrulline (NMeCit), Nα-methylhomocitrulline (Nα-MeHoCit), ornithine (Orn), Nα-Methylornithine (Nα-MeOrn or NMeOrn), sarcosine (Sar), homolysine (hLys or hK), homoarginine (hArg or hR), homoglutamine (hQ), Nα-methylarginine (NMeR), Nα-methylleucine (Nα-MeL or NMeL), N-methylhomolysine (NMeHoK), Nα-methylglutamine (NMeQ), norleucine (Nle), norvaline (Nva), 1,2,3,4-tetrahydroisoquinoline (Tic), Octahydroindole-2-carboxylic acid (Oic), 3-(1-naphthyl)alanine (1-Nal), 3-(2-naphthyl)alanine (2-Nal), 1,2,3,4-tetrahydroisoquinoline (Tic), 2-indanylglycine (IgI), para-iodophenylalanine (pI-Phe), para-aminophenylalanine (4AmP or 4-Amino-Phe), 4-guanidino phenylalanine (Guf), glycyllysine (abbreviated “K(Nε-glycyl)” or “K(glycyl)” or “K(gly)”), nitrophenylalanine (nitrophe), aminophenylalanine (aminophe or Amino-Phe), benzylphenylalanine (benzylphe), γ-carboxyglutamic acid (γ-carboxyglu), hydroxyproline (hydroxypro), p-carboxyl-phenylalanine (Cpa), α-aminoadipic acid (Aad), Nα-methyl valine (NMeVal), N-α-methyl leucine (NMeLeu), Nα-methylnorleucine (NMeNle), cyclopentylglycine (Cpg), cyclohexylglycine (Chg), acetylarginine (acetylarg), α, β-diaminopropionoic acid (Dpr), α,γ-diaminobutyric acid (Dab), diaminopropionic acid (Dap), cyclohexylalanine (Cha), 4-methyl-phenylalanine (MePhe), β,β-diphenyl-alanine (BiPhA), aminobutyric acid (Abu), 4-phenyl-phenylalanine (or biphenylalanine; 4Bip), α-amino-isobutyric acid (Aib), beta-alanine, beta-aminopropionic acid, piperidinic acid, aminocaprioic acid, aminoheptanoic acid, aminopimelic acid, desmosine, diaminopimelic acid, N-ethylglycine, N-ethylaspargine, hydroxylysine, allo-hydroxylysine, isodesmosine, allo-isoleucine, N-methylglycine, N-methylisoleucine, N-methylvaline, 4-hydroxyproline (Hyp), γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-methylarginine, 4-Amino-O-Phthalic Acid (4APA), and other similar amino acids, and derivatized forms of any of those specifically listed.

### Uses

The antigen binding proteins provided herein offer therapeutic benefit for the range of conditions which benefit from anti-CHRDL-1 therapy. These include, but are not limited to, including diabetes (including type 2 diabetes), as well as diabetes-related disorders including but not limited to diabetic retinopathy and diabetic nephropathy, and insulin resistance. Additional therapeutic benefit is for treatment of obesity, dyslipidemia, NASH, cardiovascular disease (e.g., coronary heart disease, CHF), as well as related diseases and conditions such as hypercholesterolemia and hypertriglyceridemia. Additionally, inhibiting CHRDL-1 can be beneficial in treating kidney disease. It will be appreciated that any disease or condition in which it is desirable to inhibit or otherwise modulate CHRDL-1, will also be treatable by the antigen binding proteins described herein.

Certain antigen binding proteins described herein are antibodies or are derived from antibodies. In certain embodiments, the polypeptide structure of the antigen binding proteins is based on antibodies, including, but not limited to, monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), hemibodies and fragments thereof. The various structures are further described herein below.

The antigen binding proteins that specifically bind to CHRDL-1 disclosed herein have a variety of utilities. Some of the antigen binding proteins, for instance, are useful in specific binding assays, in the affinity purification of CHRDL-1, and in screening assays to identify other antagonists or modulators of CHRDL-1 activity.

The antigen binding proteins described herein can be used in a variety of treatment applications. For example, certain antigen binding proteins are useful for treating conditions associated with CHRDL-1 activity processes in a patient, such as reducing, alleviating, or treating diabetes, obesity, dyslipidemia, NASH, cardiovascular disease, and metabolic syndrome. Other uses for the antigen binding proteins include, for example, diagnosis of diseases or conditions associated with CHRDL-1, and screening assays to determine the presence or absence of these molecules.

### CHRDL-1 Sequences

The amino acid and nucleotide sequences encoding murine and human CHRDL-1 are provided herein.

Human CHRDL-1 Protein
(SEQ ID NO: 1)
MGGMKYIFSLLFFLLLEGGKTEQVKHSETYCMFQDKKYRVGERWHPYLEP
YGLVYCVNCICSENGNVLCSRVRCPNVHCLSPVHIPHLCCPRCPDSLPPV
NNKVTSKSCEYNGTTYQHGELFVAEGLFQNRQPNQCTQCSCSEGNVYCGL
KTCPKLTCAFPVSVPDSCCRVCRGDGELSWEHSDGDIFRQPANREARHSY
HRSHYDPPPSRQAGGLSRFPGARSHRGALMDSQQASGTIVQIVINNKHKH
GQVCVSNGKTYSHGESWHPNLRAFGIVECVLCTCNVTKQECKKIHCPNRY
PCKYPQKIDGKCCKVCPGKKAKELPGQSFDNKGYFCGEETMPVYESVFME
DGETTRKIALETERPPQVEVHVWTIRKGILQHFHIEKISKRMFEELPHFK
LVTRTTLSQWKIFTEGEAQISQMCSSRVCRTELEDLVKVLYLERSEKGHC
Human CHRDL-1 Open Reading Frame cDNA
(SEQ ID NO: 2)
atgggaggcatgaaatacatatttcgttgttgttattatttgctagaagg
aggcaaaacagagcaagtaaaacattcagagacatattgcatgtttcaag
acaagaagtacagagtgggtgagagatggcatccttacctggaaccttat
gggttggtttactgcgtgaactgcatctgctcagagaatgggaatgtgct
ttgcagccgagtcagatgtccaaatgttcattgcctttctcctgtgcata
ttcctcatctgtgctgccctcgctgcccagactccttacccccagtgaac
aataaggtgaccagcaagtcttgcgagtacaatgggacaacttaccaaca
tggagagagttcgtagagaagggctattcagaatcggcaacccaatcaat
gcacccagtgcagagttcggagggaaacgtgtattgtggtctcaagactt
gccccaaattaacctgtgccttcccagtctctgttccagattcctgctgc
cgggtatgcagaggagatggagaactgtcatgggaacattctgatggtga
tatcttccggcaacctgccaacagagaagcaagacattcttaccaccgct
ctcactatgatcctccaccaagccgacaggctggaggtctgtcccgcttt
cctggggccagaagtcaccggggagacttatggattcccagcaagcatca
ggaaccattgtgcaaattgtcatcaataacaaacacaagcatggacaagt
gtgtgtttccaatggaaagacctattctcatggcgagtcctggcacccaa
acctccgggcatttggcattgtggagtgtgtgctatgtacttgtaatgtc
accaagcaagagtgtaagaaaatccactgccccaatcgatacccctgcaa
gtatcctcaaaaaatagacggaaaatgctgcaaggtgtgtccaggtaaaa
aagcaaaagaacttccaggccaaagattgacaataaaggctacttctgcg
gggaagaaacgatgcctgtgtatgagtctgtattcatggaggatggggag
acaaccagaaaaatagcactggagactgagagaccacctcaggtagaggt
ccacgtttggactattcgaaagggcattaccagcacttccatattgagaa
gatctccaagaggatgtttgaggagcttcctcacttcaagaggtgaccag
aacaaccctgagccagtggaagatcttcaccgaaggagaagctcagatca
gccagatgtgttcaagtcgtgtatgcagaacagagcttgaagatttagtc
aaggttttgtacctggagagatctgaaaagggccactgttag
Mouse CHRDL-1 Protein Sequence
(SEQ ID NO: 3)

| | |
|---|---|
| 1 | EQVKHSDTYCVFQDKKYRVGEKWHPYLEPYGLVYCVNCICSENGNV |
| | LCSR |
| 51 | VRCPSLHCLSPVHIPHLCCPRCPDSLPPVNNKVTSKSCEYNGTTYQ |
| | HGEL |
| 101 | FIAEGLFQNRQPNQCSQCSCSEGNVYCGLKTCPKLTCAFPVSVPDS |
| | CCRV |
| 151 | CRGDAELSWEHADGDIFRQPANREARHSYLRSPYDPPPNRQAGGLP |
| | RFPG |
| 201 | SRSHRGAVIDSQQASGTIVQIVINNKHKHGQVCVSNGKTYSHGESW |
| | HPNL |
| 251 | RAFGIVECVLCTCNVTKQECKKIHCPNRYPCKYPQKIDGKCCKVCP |
| | EEPP |
| 301 | SQNFDSKGSFCGEETMPVYESVFMEDGETTRKVALETERPPQVEVH |
| | VWTI |
| 351 | QKGILQHFHIEKISKRMFGELHHFKLVTRTTLNQWKLFTEGEAQLS |
| | QMCS |
| 401 | SQVCRTELEDLVQVLYLGRPEKDHC |

Mouse CHRDL-1 cDNA Sequence
(SEQ ID NO: 4)

| | |
|---|---|
| 1 | gaacaagtaaaacactcagacacatattgcgtgtttcaagacaaga |
| | agta |
| 51 | tagagtgggtgagaaatggcatccctacctggaaccgtatggactg |
| | gttt |
| 101 | actgtgtgaactgcatctgctctgagaatgggaatgtgctttgcag |
| | ccga |
| 151 | gtcagatgtccaagtcttcattgcctttcacccgtgcatattcctc |
| | atct |
| 201 | ctgttgcccccgctgcccagactccttaccaccagtgaacaataag |
| | gtga |
| 251 | ccagcaagtcatgcgaatacaatggaaccacttaccaacatggaga |
| | actg |
| 301 | ttcatagctgaagggctctttcagaaccggcaacccaatcagtgca |
| | gtca |
| 351 | gtgtagctgctcggaggggaatgtatactgtggtctcaagacttgc |
| | ccca |
| 401 | aactgacctgtgcattcccagtctctgttccagattcttgctgccg |
| | agta |
| 451 | tgcagaggggatgcagaattatcgtgggaacatgcggatggtgata |
| | tctt |
| 501 | ccggcaacctgccaacagagaagcaagacattcttacctccgttcc |
| | ccct |
| 551 | acgatcctccaccaaacagacaagctggaggtcttccccgctttcc |
| | tggg |
| 601 | agcagaagtcaccggggagctgttatagattcccagcaagcatccg |
| | ggac |
| 651 | catcgtgcagattgtcatcaataacaagcacaaacatggacaagtg |
| | tgtg |
| 701 | tttccaatggaaagacctactctcatggagagtcctggcacccaaa |
| | tcta |
| 751 | cgagcatttggcattgtggaatgtgtactatgcacttgtaatgtca |
| | ccaa |
| 801 | gcaagaatgtaagaaaatccactgccccaatcgatacccctgcaag |
| | tatc |
| 851 | ctcaaaaaatagatggaaagtgctgcaaggtgtgcccagaagaacc |
| | tcca |
| 901 | agccaaaactttgacagcaaaggttccttttgtggagaagaaacca |
| | tgcc |
| 951 | tgtatatgagtctgtgttcatggaggatggagagacaaccagaaaa |
| | gtag |
| 1001 | cactggagaccgagagaccacctcaagtagaggtccacgtttggac |
| | tatt |
| 1051 | caaaagggcattctccagcacttccacattgagaagatttccaaga |
| | ggat |
| 1101 | gtttggggagctccatcatttcaagctagttactcggaccaccttg |
| | aacc |
| 1151 | agtggaagctcttcactgaaggagaagctcagctcagccagatgtg |
| | ctca |
| 1201 | agtcaggtgtgcagaacagagctggaagatttagtccaggttttgt |
| | acct |
| 1251 | ggggagacctgaaaaggaccactgt |

A variety of antigen binding proteins useful for modulating CHRDL-1 activity are provided herein. These agents include, for example, antibodies in the traditional sense. Additionally, for instance, antigen binding proteins may contain one or more antigen binding domains (e.g., single chain antibodies, domain antibodies, hemibodies, immunoadhesions, and polypeptides with an antigen binding region) and specifically bind to CHRDL-1.

In general, the antigen binding proteins that are provided typically comprise one or more CDRs as described herein (e.g., 1, 2, 3, 4, 5, or 6 CDRs). In some embodiments the antigen binding proteins are naturally expressed by clones, while in other embodiments, the antigen binding protein can comprise (a) a polypeptide framework structure and (b) one or more CDRs that are inserted into and/or joined to the polypeptide framework structure. In some of these embodiments a CDR forms a component of a heavy or light chains expressed by the clones described herein; in other embodiments a CDR can be inserted into a framework in which the CDR is not naturally expressed. A polypeptide framework structure can take a variety of different forms. For example, a polypeptide framework structure can be, or comprise, the framework of a naturally occurring antibody, or fragment or variant thereof, or it can be completely synthetic in nature. Examples of various antigen binding protein structures are further described below.

In some embodiments in which the antigen binding protein comprises (a) a polypeptide framework structure and (b) one or more CDRs that are inserted into and/or joined to the polypeptide framework structure, the polypeptide framework structure of an antigen binding protein is an antibody or is derived from an antibody, including, but not limited to, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and portions or fragments of each, respectively. In some instances, the antigen binding protein is an immunological fragment of an antibody (e.g., a Fab, a Fab′, a F(ab′)₂, or a scFv).

In one embodiment, an antigen binding protein specifically binds to human CHRDL-1 (SEQ ID NO: 1). In another embodiment, the antigen binding protein specifically binds to murine CHRDL-1 (SEQ ID NO: 3). In yet another embodiment, the antigen binding protein specifically binds to both.

### Antigen Binding Protein Structure

Some of the antigen binding proteins that specifically bind to CHRDL-1 provided herein have a structure typically associated with naturally occurring antibodies. The structural units of these antibodies typically comprise one or more tetramers, each composed of two identical couplets of polypeptide chains, though some species of mammals also produce antibodies having only a single heavy chain. In a typical antibody, each pair or couplet includes one full-length “light” chain (in certain embodiments, about 25 kDa) and one full-length “heavy” chain (in certain embodiments, about 50-70 kDa). Each individual immunoglobulin chain is composed of several “immunoglobulin domains,” each consisting of roughly 90 to 110 amino acids and expressing a characteristic folding pattern. These domains are the basic units of which antibody polypeptides are composed. The amino-terminal portion of each chain typically includes a variable domain that is responsible for antigen recognition. The carboxy-terminal portion is more conserved evolutionarily than the other end of the chain and is referred to as the “constant region” or “C region”. Human light chains generally are classified as kappa (“κ”) and lambda (“λ”) light chains, and each of these contains one variable domain and one constant domain. Heavy chains are typically classified as mu, delta, gamma, alpha, or epsilon chains, and these define the antibody's isotype as IgM, IgD, IgG, IgA, and IgE, respectively. IgG has several subtypes, including, but not limited to, IgG1, IgG2, IgG3, and IgG4. IgM subtypes include IgM, and IgM2. IgA subtypes include IgA1 and IgA2. In humans, the IgA and IgD isotypes contain four heavy chains and four light chains; the IgG and IgE isotypes contain two heavy chains and two light chains; and the IgM isotype contains five heavy chains and five light chains. The heavy chain C region typically comprises one or more domains that can be responsible for effector function. The number of heavy chain constant region domains will depend on the isotype. IgG heavy chains, for example, each contain three C region domains known as C_{H}1, C_{H}2 and C_{H}3. The antibodies that are provided can have any of these isotypes and subtypes. In certain embodiments, an antigen binding protein that specifically binds to CHRDL-1 is an antibody of the IgG1, IgG2, or IgG4 subtype. They may also be of the IgG3 or IgG5 subtype.

In full-length light and heavy chains, the variable and constant regions are joined by a “J” region of about twelve or more amino acids, with the heavy chain also including a “D” region of about ten more amino acids. See e.g., Fundamental Immunology, 2nd ed., Ch. 7 (Paul, W., ed.) 1989, New York: Raven Press (hereby incorporated by reference in its entirety for all purposes). The variable regions of each light/heavy chain pair typically form the antigen binding site.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain/light chain pair mentioned above typically are aligned by the framework regions to form a structure that binds specifically with a specific epitope on the target protein (e.g., CHRDL-1). From N-terminal to C-terminal, naturally-occurring light and heavy chain variable regions both typically conform with the following order of these elements: FR1, CDR1, FR2, CDR2, FR3, CDR3 and FR4. A numbering system has been devised for assigning numbers to amino acids that occupy positions in each of these domains. This numbering system is defined in Kabat et al., (1991) “Sequences of Proteins of Immunological Interest”, 5^{th} Ed., U.S. Dept. of Health and Human Services, PHS, NIH, NIH Publication no. 91-3242. Although presented using the Kabat nomenclature system, as desired, the CDRs disclosed herein can also be redefined according an alternative nomenclature scheme, such as that of Chothia (see Chothia & Lesk, (1987) J. Mol. Biol. 196:901-917; Chothia et al., (1989) Nature 342:878-883 or Honegger & Pluckthun, (2001) J. Mol. Biol. 309:657-670).

The various heavy chain and light chain variable regions of antigen binding proteins are provided in FIGS. 1A-1B, 2, 7, and 8. Each of these variable regions can be attached to the disclosed heavy and light chain constant regions to form a complete antibody heavy and light chain, respectively. Further, each of the so-generated heavy and light chain sequences can be combined to form a complete antibody structure. It should be understood that the heavy chain and light chain variable regions provided herein can also be attached to other constant domains having different sequences than the exemplary sequences listed above.

CHRDL-1 antibodies and/or binding proteins of the present invention preferably inhibit chordin-like 1 function and/or activity in the cell-based assay described herein and/or cross-block the binding to CHRDL-1 of one of the antibodies described in this application and/or are cross-blocked from binding to chordin-like 1 by one of the antibodies described in this application and/or improve metabolic-related parameters in the in vivo assay described herein. Accordingly such antibodies and/or binding proteins can be identified using the assays described herein.

Specific examples of some of the full length light and heavy chains of the antibodies that are provided and their corresponding amino acid sequences are summarized in FIGS. 1A and 1B. FIG. 1A shows exemplary light chain CDR sequences, and FIG. 1B shows exemplary heavy chain sequences. “LC” refers to light chain and “HC” refers to heavy chain. Antibody “TC1E3.1” is a mouse IgG1 HC and kappa LC antibody. Antibody “TC2E1.1” is a mouse IgG1 HC and kappa LC antibody. Variable domain sequences of exemplary anti-CHRDL-1 antibodies are set forth in FIG. 2.

In some instances, the antibodies comprise two different heavy chains and two different light chains listed in the Figures infra. In other instances, the antibodies contain two identical light chains and two identical heavy chains.

In another aspect of the instant disclosure, “hemibodies” are provided. A hemibody is a monovalent antigen binding protein comprising (i) an intact light chain, and (ii) a heavy chain fused to an Fc region (e.g., an IgG2 Fc region), optionally via a linker, The linker can be a (G₄S)ₓ linker where “x” is a non-zero integer (e.g., (G₄S)₂, (G₄S)₃, (G₄S)₄, (G₄S)₅, (G₄S)₆, (G₄S)₇, (G₄S)₈, (G₄S)₉, (G₄S)₁₀, respectively). Hemibodies can be constructed using the provided heavy and light chain components.

Other antigen binding proteins that are provided are variants of antibodies formed by combination of the CDRs, and/or heavy and light chains shown in FIGS. 1A, 1B, 2, 7, and 8 herein and comprise light and/or heavy chains that each have at least 70%, 75%, 80%, 85%, 90%, 95%, 96%, 97%, 98% or 99% identity to the amino acid sequences of these chains. In some instances, such antibodies include at least one heavy chain and one light chain, whereas in other instances the variant forms contain two identical light chains and two identical heavy chains.

In some instances, the antigen binding proteins can comprise amino acid sequences that have 70%, 75%, 80%, 85%, 90%, 95%, 96%, 97%, 98% or 99% sequence identity with the CDRs described herein. In some instances, the antigen binding proteins can comprise amino acid sequences that have 70%, 75%, 80%, 85%, 90%, 95%, 96%, 97%, 98% or 99% sequence identity with the herein described variable domains. In some instances, the antigen binding proteins comprise amino acid sequences that have 70%, 75%, 80%, 85%, 90%, 95%, 96%, 97%, 98% or 99% sequence identity with the described antibodies.

Still other antigen binding proteins, e.g., antibodies or immunologically functional fragments, include variant forms of a variant heavy chain and a variant light chain as just described.

### Antigen Binding Protein CDRs

In various embodiments, the antigen binding proteins disclosed herein can comprise polypeptides into which one or more CDRs are grafted, inserted and/or joined. An antigen binding protein can have 1, 2, 3, 4, 5, or 6 CDRs. An antigen binding protein thus can have, for example, one heavy chain CDR1 (“CDRH1”), and/or one heavy chain CDR2 (“CDRH2”), and/or one heavy chain CDR3 (“CDRH3”), and/or one light chain CDR1 (“CDRL1”), and/or one light chain CDR2 (“CDRL2”), and/or one light chain CDR3 (“CDRL3”). Some antigen binding proteins include both a CDRH3 and a CDRL3. Specific heavy and light chain CDRs are identified herein.

Complementarity determining regions (CDRs) and framework regions (FR) of a given antibody can be identified using the system described by Kabat et al., (1991) “Sequences of Proteins of Immunological Interest”, 5^{th} Ed., U.S. Dept. of Health and Human Services, PHS, NIH, NIH Publication no. 91-3242. Although presented in the Kabat nomenclature scheme, as desired, the CDRs disclosed herein can also be redefined according an alternative nomenclature scheme, such as that of Chothia (see Chothia & Lesk, (1987) J. Mol. Biol. 196:901-917; Chothia et al., (1989) Nature 342:878-883 or Honegger & Pluckthun, (2001) J. Mol. Biol. 309:657-670). Certain antibodies that are disclosed herein comprise one or more amino acid sequences that are identical or have substantial sequence identity to the amino acid sequences of one or more of the CDRs presented in FIGS. 1A, 1B, 2, 7, and 8 herein.

The structure and properties of CDRs within a naturally occurring antibody has been described, supra. Briefly, in a traditional antibody, the CDRs are embedded within a framework in the heavy and light chain variable region where they constitute the regions responsible for antigen binding and recognition. A variable region comprises at least three heavy or light chain CDRs, see e.g., Kabat et al., (1991) “Sequences of Proteins of Immunological Interest”, 5^{th} Ed., U.S. Dept. of Health and Human Services, PHS, NIH, NIH Publication no. 91-3242; see also Chothia and Lesk, (1987) J. Mol. Biol. 196:901-917; Chothia et al., (1989) Nature 342: 877-883), within a framework region (designated framework regions 1-4, FR1, FR2, FR3, and FR4, by Kabat et al., (1991); see also Chothia and Lesk, (1987) supra). The CDRs provided herein, however, can not only be used to define the antigen binding domain of a traditional antibody structure, but can be embedded in a variety of other polypeptide structures, as described herein.

In one aspect, the antigen binding proteins contain one or more amino acid substitutions, deletions or insertions. In another aspect, the antigen binding protein has at least 80%, 85%, 90%, 95%, 96%, 97%, 98%, or 99% sequence identity to a CDR sequence listed in FIGS. 1A, 1B, 2, 7, and 8 herein. In another aspect, the antigen binding protein has at least 80%, 85%, 90%, 95%, 96%, 97%, 98%, or 99% sequence identity to the variable region sequences listed in FIGS. 27, and 8 herein.

### Consensus Sequences

In yet another aspect, the CDRs disclosed herein include consensus sequences derived from groups of related monoclonal antibodies. As described herein, a “consensus sequence” refers to amino acid sequences having conserved amino acids common among a number of sequences and variable amino acids that vary within a given amino acid sequences.

In one aspect, also provided is an antigen binding protein that specifically binds to a linear or three-dimensional epitope comprising one or more amino acid residues from CHRDL-1.

In another embodiment, the antibody fragment of the isolated antigen-binding proteins provided herein can be a Fab fragment, a Fab′ fragment, an F(ab′)₂ fragment, an Fv fragment, a diabody, or a single chain antibody molecule.

In a further embodiment, an isolated antigen binding protein that specifically binds to CHRDL-1 provided herein is a human antibody and can be of the IgG1-, IgG2- IgG3- or IgG4-type.

Such antigen binding proteins, and indeed any of the antigen binding proteins disclosed herein, can also be PEGylated with one or more PEG molecules, for examples PEG molecules having a molecular weight selected from the group consisting of 5K, 10K, 20K, 40K, 50K, 60K, 80K, 100K, or greater than 100K.

In one embodiment, the isolated antigen binding protein provided herein can reduce blood glucose levels, decrease triglyceride and cholesterol levels or improve other glycemic parameters and cardiovascular risk factors when administered to a patient.

As will be appreciated, for any antigen binding protein comprising more than one CDR provided in FIGS. 1A, 1B, 2, 7, and 8 herein, any combination of CDRs independently selected from the depicted sequences may be useful. Thus, antigen binding proteins with one, two, three, four, five or six of independently selected CDRs can be generated. However, as will be appreciated by those in the art, specific embodiments generally utilize combinations of CDRs that are non-repetitive, e.g., antigen binding proteins are generally not made with two CDRH2 regions, etc.

### Antigen Binding Proteins and Binding Epitopes and Binding Domains

When an antigen binding protein is said to bind an epitope on CHRDL-1, what is meant is that the antigen binding protein specifically binds to a specified portion of CHRDL-1. In some embodiments, e.g., the antigen binding protein can specifically bind to a polypeptide consisting of specified residues of CHRDL-1. In any of these embodiments, such an antigen binding protein does not need to contact every residue of CHRDL-1, nor does every single amino acid substitution or deletion within CHRDL-1 necessarily significantly affect binding affinity.

Epitope specificity and the binding domain(s) of an antigen binding protein can be determined by a variety of methods. Some methods, for example, can use truncated portions of an antigen. Other methods utilize antigen mutated at one or more specific residues, such as by employing an alanine scanning or arginine scanning-type approach or by the generation and study of chimeric proteins in which various domains, regions or amino acids are swapped between two proteins (e.g., mouse and human forms of one or more of the antigens or target proteins), or by protease protection assays.

### Competing Antigen Binding Proteins

In another aspect, antigen binding proteins are provided that compete with one of the exemplified antibodies or functional fragments for binding to CHRDL-1. Such antigen binding proteins can also bind to the same epitope as one of the herein exemplified antigen binding proteins, or an overlapping epitope. Antigen binding proteins and fragments that compete with or bind to the same epitope as the exemplified antigen binding proteins are expected to show similar functional properties. The ability to compete with an antibody can be determined using any suitable assay, such as those described herein.

### Monoclonal Antibodies

The antigen binding proteins that are provided include monoclonal antibodies that bind to CHRDL-1 to various degrees. Monoclonal antibodies can be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in mouse fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XXO Bul; examples of cell lines used in rat fusions include R210.RCY3, Y3-Ag 1.2.3, IR983F and 4B210. Other cell lines useful for cell fusions are U-266, GM1500-GRG2, LICR-LON-HMy2 and UC729-6.

In some instances, a hybridoma cell line is produced by immunizing an animal (e.g., a transgenic animal having human immunoglobulin sequences) with an immunogen comprising (1) full length human CHRDL-1, obtained by transfecting CHO cells with cDNA encoding a human full length CHRDL-1 of SEQ ID NO: 1; harvesting spleen cells from the immunized animal; fusing the harvested spleen cells to a myeloma cell line, thereby generating hybridoma cells; establishing hybridoma cell lines from the hybridoma cells, and identifying a hybridoma cell line that produces an antibody that binds to CHRDL-1. Such hybridoma cell lines, and the monoclonal antibodies produced by them, form aspects of the present disclosure. Monoclonal antibodies secreted by a hybridoma cell line can be purified using any technique known in the art. Hybridomas or mAbs can be further screened to identify mAbs with particular desired properties, such as the ability to inhibit CHRDL-1 activity and/or signaling. Examples of such screens are provided herein.

### Chimeric and Humanized Antibodies

Chimeric and humanized antibodies based upon the sequences herein can readily be generated. One example is a chimeric antibody, which is an antibody composed of protein segments from different antibodies that are covalently joined to produce functional immunoglobulin light or heavy chains or immunologically functional portions thereof. Generally, a portion of the heavy chain and/or light chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. For methods relating to chimeric antibodies, see, for example, U.S. Pat. No. 4,816,567; and Morrison et al., PNAS USA 81:6851-6855 (1985), which are hereby incorporated by reference. CDR grafting is described, for example, in U.S. Pat. No. 6,180,370, U.S. Pat. No. 5,693,762, U.S. Pat. No. 5,693,761, U.S. Pat. No. 5,585,089, and U.S. Pat. No. 5,530,101.

It will be appreciated that humanized antibodies are produced from one or more monoclonal antibodies raised initially in a non-human animal. Certain amino acid residues in these monoclonal antibodies typically, from non-antigen recognizing portions of the antibody, are modified to be homologous to corresponding residues in a human antibody of corresponding isotype. Humanization can be performed, for example, using various methods by substituting at least a portion of a rodent variable region for the corresponding regions of a human antibody (See e.g., U.S. Pat. No. 5,585,089, and No. 5,693,762; Jones et al., (1986) Nature 321:522-525; Riechmann et al., (1988) Nature 332:323-27; Verhoeyen et al., (1988) Science 239:1534-1536).

To create consensus human FRs, FRs from several human heavy chain or light chain amino acid sequences can be aligned to identify a consensus amino acid sequence. In other embodiments, the FRs of a heavy chain or light chain disclosed herein are replaced with the FRs from a different heavy chain or light chain. In one aspect, rare amino acids in the FRs of the heavy and light chains of an antigen binding protein (e.g., an antibody) that specifically binds to CHRDL-1 are not replaced, while the rest of the FR amino acids are replaced. A “rare amino acid” is a specific amino acid that is in a position in which this particular amino acid is not usually found in an FR. Alternatively, the grafted variable regions from the one heavy or light chain can be used with a constant region that is different from the constant region of that particular heavy or light chain as disclosed herein. In other embodiments, the grafted variable regions are part of a single chain Fv antibody. In certain embodiments, constant regions from species other than human can be used along with the human variable region(s) to produce hybrid antibodies.

### Fully Human Antibodies

Fully human antibodies are provided by the instant disclosure. Methods are available for making fully human antibodies specific for a given antigen without exposing human beings to the antigen (“fully human antibodies”). One specific means provided for implementing the production of fully human antibodies is the “humanization” of the mouse humoral immune system. Introduction of human immunoglobulin (Ig) loci into mice in which the endogenous Ig genes have been inactivated is one means of producing fully human monoclonal antibodies (mAbs) in mouse, an animal that can be immunized with any desirable antigen. Using fully human antibodies can minimize the immunogenic and allergic responses that can sometimes be caused by administering mouse or mouse-derived mAbs to humans as therapeutic agents.

Fully human antibodies can be produced by immunizing transgenic animals (typically mice) that are capable of producing a repertoire of human antibodies in the absence of endogenous immunoglobulin production. Antigens for this purpose typically have six or more contiguous amino acids, and optionally are conjugated to a carrier, such as a hapten. See e.g., Jakobovits et al., (1993) Proc. Natl. Acad. Sci. USA 90:2551-2555; Jakobovits et al., (1993) Nature 362:255-258; and Bruggermann et al., (1993) Year in Immunol. 7:33. In one example of such a method, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then cross-bred to obtain an animal having all of the desired immune system modifications. When administered an immunogen, these transgenic animals produce antibodies that are immunospecific for the immunogen but have human rather than murine amino acid sequences, including the variable regions. For further details of such methods, e.g., WO96/33735 and WO94/02602. Additional methods relating to transgenic mice for making human antibodies are described in U.S. Pat. No. 5,545,807; No. 6,713,610; No. 6,673,986; No. 6,162,963; No. 5,545,807; No. 6,300,129; No. 6,255,458; No. 5,877,397; No. 5,874,299 and No. 5,545,806; in PCT publications WO91/10741, WO90/04036, and in EP 546073 and EP 546073.

According to certain embodiments, antibodies of the invention can be prepared through the utilization of a transgenic mouse that has a substantial portion of the human antibody producing genome inserted but that is rendered deficient in the production of endogenous, murine antibodies. Such mice, then, are capable of producing human immunoglobulin molecules and antibodies and are deficient in the production of murine immunoglobulin molecules and antibodies. Technologies utilized for achieving this result are disclosed in the patents, applications and references disclosed in the specification, herein. In certain embodiments, one can employ methods such as those disclosed in PCT Published Application No. WO 98/24893 or in Mendez et al., (1997) Nature Genetics, 15:146-156, which are hereby incorporated by reference for any purpose.

Generally, fully human monoclonal antibodies specific for CHRDL-1 can be produced as follows. Transgenic mice containing human immunoglobulin genes are immunized with the antigen of interest, e.g. those described herein, lymphatic cells (such as B-cells) from the mice that express antibodies are obtained. Such recovered cells are fused with a myeloid-type cell line to prepare immortal hybridoma cell lines, and such hybridoma cell lines are screened and selected to identify hybridoma cell lines that produce antibodies specific to the antigen of interest. In certain embodiments, the production of a hybridoma cell line that produces antibodies specific to CHRDL-1.

In certain embodiments, fully human antibodies can be produced by exposing human splenocytes (B or T cells) to an antigen in vitro, and then reconstituting the exposed cells in an immunocompromised mouse, e.g. SCID or nod/SCID. See e.g., Brams et al., J. Immunol. 160: 2051-2058 (1998); Carballido et al., Nat. Med., 6: 103-106 (2000). In certain such approaches, engraftment of human fetal tissue into SCID mice (SCID-hu) results in long-term hematopoiesis and human T-cell development. See e.g., McCune et al., Science, 241:1532-1639 (1988); Ifversen et al., Sem. Immunol., 8:243-248 (1996). In certain instances, humoral immune response in such chimeric mice is dependent on co-development of human T-cells in the animals. See e.g., Martensson et al., Immunol., 83:1271-179 (1994). In certain approaches, human peripheral blood lymphocytes are transplanted into SCID mice. See e.g., Mosier et al., Nature, 335:256-259 (1988). In certain such embodiments, when such transplanted cells are treated either with a priming agent, such as Staphylococcal Enterotoxin A (SEA), or with anti-human CD40 monoclonal antibodies, higher levels of B cell production is detected. See e.g., Martensson et al., Immunol., 84: 224-230 (1995); Murphy et al., Blood, 86:1946-1953 (1995).

Thus, in certain embodiments, fully human antibodies can be produced by the expression of recombinant DNA in host cells or by expression in hybridoma cells. In other embodiments, antibodies can be produced using the phage display techniques described herein.

The antibodies described herein were prepared through the utilization of the XenoMouse® technology, as described herein. Such mice, then, are capable of producing human immunoglobulin molecules and antibodies and are deficient in the production of murine immunoglobulin molecules and antibodies. Technologies utilized for achieving the same are disclosed in the patents, applications, and references disclosed in the background section herein. In particular, however, a preferred embodiment of transgenic production of mice and antibodies therefrom is disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. See also Mendez et al., Nature Genetics, 15:146-156 (1997), the disclosure of which is hereby incorporated by reference.

Through the use of such technology, fully human monoclonal antibodies to a variety of antigens have been produced. Essentially, XenoMouse® lines of mice are immunized with an antigen of interest (e.g. an antigen provided herein), lymphatic cells (such as B-cells) are recovered from the hyper-immunized mice, and the recovered lymphocytes are fused with a myeloid-type cell line to prepare immortal hybridoma cell lines. These hybridoma cell lines are screened and selected to identify hybridoma cell lines that produced antibodies specific to the antigen of interest. Provided herein are methods for the production of multiple hybridoma cell lines that produce antibodies specific to CHRDL-1. Further, provided herein are characterization of the antibodies produced by such cell lines, including nucleotide and amino acid sequence analyses of the heavy and light chains of such antibodies.

The production of the XenoMouse® strains of mice is further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, Ser. No. 08/031,801, filed Mar. 15, 1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430,938, filed Apr. 27, 1995, Ser. No. 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, Ser. No. 08/759,620, filed Dec. 3, 1996, U.S. Publication 2003/0093820, filed Nov. 30, 2001 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also European Patent No., EP 0 463 151 B1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications, and references are hereby incorporated by reference in their entirety.

Using hybridoma technology, antigen-specific human mAbs with the desired specificity can be produced and selected from the transgenic mice such as those described herein. Such antibodies can be cloned and expressed using a suitable vector and host cell, or the antibodies can be harvested from cultured hybridoma cells.

Fully human antibodies can also be derived from phage-display libraries (as described in Hoogenboom et al., (1991) J. Mol. Biol. 227:381; and Marks et al., (1991) J. Mol. Biol. 222:581). Phage display techniques mimic immune selection through the display of antibody repertoires on the surface of filamentous bacteriophage, and subsequent selection of phage by their binding to an antigen of choice. One such technique is described in PCT Publication No. WO 99/10494 (hereby incorporated by reference), which describes the isolation of high affinity and functional agonistic antibodies for MPL- and msk-receptors using such an approach.

### Bispecific or Bifunctional Antigen Binding Proteins

Also provided are bispecific and bifunctional antibodies that include one or more CDRs or one or more variable regions as described above. A bispecific or bifunctional antibody in some instances can be an artificial hybrid antibody having two different heavy/light chain pairs and two different binding sites. Bispecific antibodies can be produced by a variety of methods including, but not limited to, fusion of hybridomas or linking of Fab′ fragments. See e.g., Songsivilai & Lachmann, (1990) Clin. Exp. Immunol. 79:315-321; Kostelny et al., (1992) J. Immunol. 148:1547-1553.

### Various Other Forms

In various embodiments, the antigen binding proteins disclosed herein can comprise one or more non-naturally occurring/encoded amino acids. For instance, some of the antigen binding proteins have one or more non-naturally occurring/encoded amino acid substitutions in one or more of the heavy or light chains, variable regions or CDRs listed herein. Examples of non-naturally occurring/encoded amino acids (which can be substituted for any naturally-occurring amino acid found in any sequence disclosed herein, as desired) include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention. A non-limiting lists of examples of non-naturally occurring/encoded amino acids that can be inserted into an antigen binding protein sequence or substituted for a wild-type residue in an antigen binding sequence include β-amino acids, homoamino acids, cyclic amino acids and amino acids with derivatized side chains. Examples include (in the L-form or D-form; abbreviated as in parentheses): citrulline (Cit), homocitrulline (hCit), Nα-methylcitrulline (NMeCit), Nα-methylhomocitrulline (Nα-MeHoCit), ornithine (Orn), Nα-Methylornithine (Nα-MeOrn or NMeOrn), sarcosine (Sar), homolysine (hLys or hK), homoarginine (hArg or hR), homoglutamine (hQ), Nα-methylarginine (NMeR), Nα-methylleucine (Nα-MeL or NMeL), N-methylhomolysine (NMeHoK), Nα-methylglutamine (NMeQ), norleucine (Nle), norvaline (Nva), 1,2,3,4-tetrahydroisoquinoline (Tic), Octahydroindole-2-carboxylic acid (Oic), 3-(1-naphthyl)alanine (1-Nal), 3-(2-naphthyl)alanine (2-Nal), 1,2,3,4-tetrahydroisoquinoline (Tic), 2-indanylglycine (IgI), para-iodophenylalanine (pI-Phe), para-aminophenylalanine (4AmP or 4-Amino-Phe), 4-guanidino phenylalanine (Guf), glycyllysine (abbreviated “K(Nε-glycyl)” or “K(glycyl)” or “K(gly)”), nitrophenylalanine (nitrophe), aminophenylalanine (aminophe or Amino-Phe), benzylphenylalanine (benzylphe), γ-carboxyglutamic acid (γ-carboxyglu), hydroxyproline (hydroxypro), p-carboxyl-phenylalanine (Cpa), α-aminoadipic acid (Aad), Nα-methyl valine (NMeVal), N-α-methyl leucine (NMeLeu), Nα-methylnorleucine (NMeNle), cyclopentylglycine (Cpg), cyclohexylglycine (Chg), acetylarginine (acetylarg), α,β-diaminopropionoic acid (Dpr), α,γ-diaminobutyric acid (Dab), diaminopropionic acid (Dap), cyclohexylalanine (Cha), 4-methyl-phenylalanine (MePhe), β,β-diphenyl-alanine (BiPhA), aminobutyric acid (Abu), 4-phenyl-phenylalanine (or biphenylalanine; 4Bip), α-amino-isobutyric acid (Aib), beta-alanine, beta-aminopropionic acid, piperidinic acid, aminocaprioic acid, aminoheptanoic acid, aminopimelic acid, desmosine, diaminopimelic acid, N-ethylglycine, N-ethylaspargine, hydroxylysine, allo-hydroxylysine, isodesmosine, allo-isoleucine, N-methylglycine, N-methylisoleucine, N-methylvaline, 4-hydroxyproline (Hyp), γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-methylarginine, 4-Amino-O-Phthalic Acid (4APA), and other similar amino acids, and derivatized forms of any of those specifically listed.

Additionally, the antigen binding proteins can have one or more conservative amino acid substitutions in one or more of the heavy or light chains, variable regions or CDRs listed in FIGS. 1A, 1B, 2, 7, and 8 herein. Naturally-occurring amino acids can be divided into classes based on common side chain properties:

1) hydrophobic: norleucine, Met, Ala, Val, Leu, Ile;

2) neutral hydrophilic: Cys, Ser, Thr, Asn, Gln;

3) acidic: Asp, Glu;

4) basic: His, Lys, Arg;

5) residues that influence chain orientation: Gly, Pro; and

6) aromatic: Trp, Tyr, Phe.

Conservative amino acid substitutions can involve exchange of a member of one of these classes with another member of the same class. Conservative amino acid substitutions can encompass non-naturally occurring/encoded amino acid residues, which are typically incorporated by chemical peptide synthesis rather than by synthesis in biological systems. These include peptidomimetics and other reversed or inverted forms of amino acid moieties.

Non-conservative substitutions can involve the exchange of a member of one of the above classes for a member from another class. Such substituted residues can be introduced into regions of the antibody that are homologous with human antibodies, or into the non-homologous regions of the molecule.

In making such changes, according to certain embodiments, the hydropathic index of amino acids can be considered. The hydropathic profile of a protein is calculated by assigning each amino acid a numerical value (“hydropathy index”) and then repetitively averaging these values along the peptide chain. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic profile in conferring interactive biological function on a protein is understood in the art (see e.g., Kyte et al., 1982, J. Mol. Biol. 157:105-131). It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included. In some aspects, those which are within ±1 are included, and in other aspects, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigen-binding or immunogenicity, that is, with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in other embodiments, those which are within ±1 are included, and in still other embodiments, those within ±0.5 are included. In some instances, one can also identify epitopes from primary amino acid sequences on the basis of hydrophilicity. These regions are also referred to as “epitopic core regions.”

Exemplary conservative amino acid substitutions are set forth in TABLE 2.

TABLE 2
Conservative Amino Acid Substitutions

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

A skilled artisan will be able to determine suitable variants of polypeptides as set forth herein using well-known techniques coupled with the information provided herein. One skilled in the art can identify suitable areas of the molecule that can be changed without destroying activity by targeting regions not believed to be important for activity. The skilled artisan also will be able to identify residues and portions of the molecules that are conserved among similar polypeptides. In further embodiments, even areas that can be important for biological activity or for structure can be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues important for activity or structure in similar proteins. One skilled in the art can opt for chemically similar amino acid substitutions for such predicted important amino acid residues.

One skilled in the art can also analyze the three-dimensional structure and amino acid sequence in relation to that structure in similar polypeptides. In view of such information, one skilled in the art can predict the alignment of amino acid residues of an antibody with respect to its three dimensional structure. One skilled in the art can choose not to make radical changes to amino acid residues predicted to be on the surface of the protein, since such residues can be involved in important interactions with other molecules. Moreover, one skilled in the art can generate test variants containing a single amino acid substitution at each desired amino acid residue. These variants can then be screened using assays for CHRDL-1 activity (including those described in the Examples provided herein) thus yielding information regarding which amino acids can be changed and which must not be changed. In other words, based on information gathered from such routine experiments, one skilled in the art can readily determine the amino acid positions where further substitutions should be avoided either alone or in combination with other mutations.

A number of scientific publications have been devoted to the prediction of secondary structure. See, Moult, (1996) Curr. Op. in Biotech. 7:422-427; Chou et al., (1974) Biochem. 13:222-245; Chou et al., (1974) Biochemistry 113:211-222; Chou et al., (1978) Adv. Enzymol. Relat. Areas Mol. Biol. 47:45-148; Chou et al., (1979) Ann. Rev. Biochem. 47:251-276; and Chou et al., (1979) Biophys. J. 26:367-384. Moreover, computer programs are currently available to assist with predicting secondary structure. One method of predicting secondary structure is based upon homology modeling. For example, two polypeptides or proteins that have a sequence identity of greater than 30%, or similarity greater than 40% can have similar structural topologies. The growth of the protein structural database (PDB) has provided enhanced predictability of secondary structure, including the potential number of folds within a polypeptide's or protein's structure. See, Holm et al., (1999) Nucl. Acid. Res. 27:244-247. It has been suggested (Brenner et al., (1997) Curr. Op. Struct. Biol. 7:369-376) that there are a limited number of folds in a given polypeptide or protein and that once a critical number of structures have been resolved, structural prediction will become dramatically more accurate.

Additional methods of predicting secondary structure include “threading” (Jones, (1997) Curr. Opin. Struct. Biol. 7:377-387; Sippl et al., (1996) Structure 4:15-19), “profile analysis” (Bowie et al., (1991) Science 253:164-170; Gribskov et al., (1990) Meth. Enzym. 183:146-159; Gribskov et al., (1987) PNAS 84:4355-4358), and “evolutionary linkage” (See, Holm, (1999) supra; and Brenner, (1997) supra).

In some embodiments, amino acid substitutions are made that: (1) reduce susceptibility to proteolysis, (2) reduce susceptibility to oxidation, (3) alter binding affinity for forming protein complexes, (4) alter ligand or antigen binding affinities, and/or (4) confer or modify other physicochemical or functional properties on such polypeptides. For example, single or multiple amino acid substitutions (in some embodiments, conservative amino acid substitutions) can be made in the naturally-occurring sequence. Substitutions can be made in that portion of the antibody that lies outside the domain(s) forming intermolecular contacts. In such embodiments, conservative amino acid substitutions can be used that do not substantially change the structural characteristics of the parent sequence (e.g., one or more replacement amino acids that do not disrupt the secondary structure that characterizes the parent or native antigen binding protein). Examples of art-recognized polypeptide secondary and tertiary structures are described in Creighton, Proteins: Structures and Molecular Properties 2^{nd} edition, 1992, W. H. Freeman & Company; Creighton, Proteins: Structures and Molecular Principles, 1984, W. H. Freeman & Company; Introduction to Protein Structure (Branden and Tooze, eds.), 2^{nd} edition, 1999, Garland Publishing; Petsko & Ringe, Protein Structure and Function, 2004, New Science Press Ltd; and Thornton et al., (1991) Nature 354:105, which are each incorporated herein by reference.

Additional preferred antibody variants include cysteine variants wherein one or more cysteine residues in the parent or native amino acid sequence are deleted from or substituted with another amino acid (e.g., serine). Cysteine variants are useful, inter alia when antibodies must be refolded into a biologically active conformation. Cysteine variants can have fewer cysteine residues than the native antibody, and typically have an even number to minimize interactions resulting from unpaired cysteines.

The heavy and light chains, variable regions domains and CDRs that are disclosed can be used to prepare polypeptides that contain an antigen binding region that can specifically bind to CHRDL-1. For example, one or more of the CDRs listed in FIGS. 1A, 1B, 2, 7, and 8 herein can be incorporated into a molecule (e.g., a polypeptide) covalently or noncovalently to make an immunoadhesion. An immunoadhesion can incorporate the CDR(s) as part of a larger polypeptide chain, can covalently link the CDR(s) to another polypeptide chain, or can incorporate the CDR(s) noncovalently. The CDR(s) enable the immunoadhesion to bind specifically to a particular antigen of interest (e.g., CHRDL-1) or an epitope thereon.

The heavy and light chains, variable regions domains and CDRs that are disclosed can be used to prepare polypeptides that contain an antigen binding region that can specifically bind to CHRDL-1. For example, one or more of the CDRs listed in FIGS. 1A, 1B, 2, 7, and 8 herein can be incorporated into a molecule (e.g., a polypeptide) that is structurally similar to a “half” antibody comprising the heavy chain, the light chain of an antigen binding protein paired with a Fc fragment so that the antigen binding region is monovalent (like a Fab fragment) but with a dimeric Fc moiety.

Mimetics (e.g., “peptide mimetics” or “peptidomimetics”) based upon the variable region domains and CDRs that are described herein are also provided. These analogs can be peptides, non-peptides or combinations of peptide and non-peptide regions. Fauchere, (1986) Adv. Drug Res. 15:29; Veber and Freidinger, (1985) TINS p. 392; and Evans et al., (1987) J. Med. Chem. 30:1229, which are incorporated herein by reference for any purpose. Peptide mimetics that are structurally similar to therapeutically useful peptides can be used to produce a similar therapeutic or prophylactic effect. Such compounds are often developed with the aid of computerized molecular modeling. Generally, peptidomimetics are proteins that are structurally similar to an antibody displaying a desired biological activity, such as here the ability to specifically bind to CHRDL-1, but have one or more peptide linkages optionally replaced by a linkage selected from: —CH₂NH—, —CH₂S—, —CH₂—CH₂—, —CH—CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) can be used in certain embodiments to generate more stable proteins. In addition, constrained peptides comprising a consensus sequence or a substantially identical consensus sequence variation can be generated by methods known in the art (Rizo and Gierasch, (1992) Ann. Rev. Biochem. 61:387), incorporated herein by reference), for example, by adding internal cysteine residues capable of forming intramolecular disulfide bridges which cyclize the peptide.

Derivatives of the antigen binding proteins that specifically bind to CHRDL-1 are also provided. The derivatized antigen binding proteins can comprise any molecule or substance that imparts a desired property to the antibody or fragment, such as increased half-life in a particular use. The derivatized antigen binding protein can comprise, for example, a detectable (or labeling) moiety (e.g., a radioactive, colorimetric, antigenic or enzymatic molecule, a detectable bead (such as a magnetic or electrodense (e.g., gold) bead), or a molecule that binds to another molecule (e.g., biotin or streptavidin), a therapeutic or diagnostic moiety (e.g., a radioactive, cytotoxic, or pharmaceutically active moiety), or a molecule that increases the suitability of the antigen binding protein for a particular use (e.g., administration to a subject, such as a human subject, or other in vivo or in vitro uses). Examples of molecules that can be used to derivatize an antigen binding protein include albumin (e.g., human serum albumin) and polyethylene glycol (PEG). Albumin-linked and PEGylated derivatives of antigen binding proteins can be prepared using techniques well known in the art. Certain antigen binding proteins include a PEGylated single chain polypeptide as described herein. In one embodiment, the antigen binding protein is conjugated or otherwise linked to transthyretin (“TTR”) or a TTR variant. The TTR or TTR variant can be chemically modified with, for example, a chemical selected from the group consisting of dextran, poly(n-vinyl pyrrolidone), polyethylene glycols, propropylene glycol homopolymers, polypropylene oxide/ethylene oxide co-polymers, polyoxyethylated polyols and polyvinyl alcohols.

Other derivatives include covalent or aggregative conjugates of the antigen binding proteins that specifically bind to CHRDL-1 with other proteins or polypeptides, such as by expression of recombinant fusion proteins comprising heterologous polypeptides fused to the N-terminus or C-terminus of an antigen binding protein to CHRDL-1. For example, the conjugated peptide can be a heterologous signal (or leader) polypeptide, e.g., the yeast alpha-factor leader, or a peptide such as an epitope tag. An antigen binding protein-containing fusion protein of the present disclosure can comprise peptides added to facilitate purification or identification of an antigen binding protein that specifically binds to CHRDL-1. An antigen binding protein that specifically binds to CHRDL-1 also can be linked to the FLAG peptide as described in Hopp et al., (1988) Bio/Technology 6:1204; and U.S. Pat. No. 5,011,912. The FLAG peptide is highly antigenic and provides an epitope reversibly bound by a specific monoclonal antibody (mAb), enabling rapid assay and facile purification of expressed recombinant protein. Reagents useful for preparing fusion proteins in which the FLAG peptide is fused to a given polypeptide are commercially available (Sigma, St. Louis, Mo.).

Multimers that comprise one or more antigen binding proteins that specifically bind to CHRDL-1 form another aspect of the present disclosure. Multimers can take the form of covalently-linked or non-covalently-linked dimers, trimers, or higher multimers. Multimers comprising two or more antigen binding proteins that bind to CHRDL-1 are contemplated for use as therapeutics, diagnostics and for other uses as well, with one example of such a multimer being a homodimer. Other exemplary multimers include heterodimers, homotrimers, heterotrimers, homotetramers, heterotetramers, etc.

One embodiment is directed to multimers comprising multiple antigen binding proteins that specifically bind to CHRDL-1 joined via covalent or non-covalent interactions between peptide moieties fused to an antigen binding protein that specifically binds to CHRDL-1. Such peptides can be peptide linkers (spacers), or peptides that have the property of promoting multimerization. Leucine zippers and certain polypeptides derived from antibodies are among the peptides that can promote multimerization of antigen binding proteins attached thereto, as described in more detail herein.

In particular embodiments, the multimers comprise from two to four antigen binding proteins that bind to CHRDL-1. The antigen binding protein moieties of the multimer can be in any of the forms described above, e.g., variants or fragments. Preferably, the multimers comprise antigen binding proteins that have the ability to specifically bind to CHRDL-1.

In one embodiment, an oligomer is prepared using polypeptides derived from immunoglobulins. Preparation of fusion proteins comprising certain heterologous polypeptides fused to various portions of antibody-derived polypeptides (including the Fc domain) has been described, e.g., by Ashkenazi et al., (1991) Proc. Natl. Acad. Sci. USA 88:10535; Byrn et al., (1990) Nature 344:677; and Hollenbaugh et al., (1992) Current Protocols in Immunology, Suppl. 4, pages 10.19.1-10.19.11.

The term “Fc polypeptide” as used herein includes native and mutein forms of polypeptides derived from the Fc region of an antibody. Truncated forms of such polypeptides containing the hinge region that promotes dimerization also are included. Fusion proteins comprising Fc moieties (and oligomers formed therefrom) offer the advantage of facile purification by affinity chromatography over Protein A or Protein G columns.

One suitable Fc polypeptide, described in PCT application WO 93/10151 and U.S. Pat. No. 5,426,048 and No. 5,262,522, is a single chain polypeptide extending from the N-terminal hinge region to the native C-terminus of the Fc region of a human IgG1 antibody. Another useful Fc polypeptide is the Fc mutein described in U.S. Pat. No. 5,457,035, and in Baum et al., (1994) EMBO J. 13:3992-4001. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

In other embodiments, the variable portion of the heavy and/or light chains of an antigen binding protein such as disclosed herein can be substituted for the variable portion of an antibody heavy and/or light chain.

Alternatively, the oligomer is a fusion protein comprising multiple antigen binding proteins that specifically bind to CHRDL-1 with or without peptide linkers (spacer peptides). Among the suitable peptide linkers are those described in U.S. Pat. No. 4,751,180 and No. 4,935,233.

Another method for preparing oligomeric derivatives comprising that antigen binding proteins that specifically bind to CHRDL-1 involves use of a leucine zipper. Leucine zipper domains are peptides that promote oligomerization of the proteins in which they are found. Leucine zippers were originally identified in several DNA-binding proteins (Landschultz et al., (1988) Science 240:1759-64), and have since been found in a variety of different proteins. Among the known leucine zippers are naturally occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al., (1994) FEBS Letters 344:191, hereby incorporated by reference. The use of a modified leucine zipper that allows for stable trimerization of a heterologous protein fused thereto is described in Fanslow et al., (1994) Semin. Immunol. 6:267-278. In one approach, recombinant fusion proteins comprising an antigen binding protein fragment or derivative that specifically binds to CHRDL-1 is fused to a leucine zipper peptide are expressed in suitable host cells, and the soluble oligomeric antigen binding protein fragments or derivatives that form are recovered from the culture supernatant.

In certain embodiments, the antigen binding protein has a K_{D} (equilibrium binding affinity) of less than 1 pM, 10 pM, 100 pM, 1 nM, 2 nM, 5 nM, 10 nM, 25 nM or 50 nM.

In another aspect the instant disclosure provides an antigen binding protein having a half-life of at least one day in vitro or in vivo (e.g., when administered to a human subject). In one embodiment, the antigen binding protein has a half-life of at least three days. In another embodiment, the antibody or portion thereof has a half-life of four days or longer. In another embodiment, the antibody or portion thereof has a half-life of eight days or longer. In another embodiment, the antibody or portion thereof has a half-life of ten days or longer. In another embodiment, the antibody or portion thereof has a half-life of eleven days or longer. In another embodiment, the antibody or portion thereof has a half-life of fifteen days or longer. In another embodiment, the antibody or antigen-binding portion thereof is derivatized or modified such that it has a longer half-life as compared to the underivatized or unmodified antibody. In another embodiment, an antigen binding protein that specifically binds to CHRDL-1 contains point mutations to increase serum half-life, such as described in WO 00/09560, published Feb. 24, 2000, incorporated by reference.

### Glycosylation

An antigen binding protein that specifically binds to CHRDL-1 can have a glycosylation pattern that is different or altered from that found in the native species. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine can also be used.

Addition of glycosylation sites to the antigen binding protein is conveniently accomplished by altering the amino acid sequence such that it contains one or more of the above-described tri-peptide sequences (for N-linked glycosylation sites). The alteration can also be made by the addition of, or substitution by, one or more serine or threonine residues to the starting sequence (for O-linked glycosylation sites). For ease, the antigen binding protein amino acid sequence can be altered through changes at the DNA level, particularly by mutating the DNA encoding the target polypeptide at preselected bases such that codons are generated that will translate into the desired amino acids.

Another means of increasing the number of carbohydrate moieties on the antigen binding protein is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) can be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in WO 87/05330 and in Aplin & Wriston, (1981) CRC Crit. Rev. Biochem. 10:259-306.

Removal of carbohydrate moieties present on the starting antigen binding protein can be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., (1987) Arch. Biochem. Biophys. 259:52-57 and by Edge et al., (1981) Anal. Biochem. 118:131-37. Enzymatic cleavage of carbohydrate moieties on polypeptides can be achieved by the use of a variety of endo- and exo-glycosidases as described by Thotakura et al., (1987) Meth. Enzymol. 138:350-59. Glycosylation at potential glycosylation sites can be prevented by the use of the compound tunicamycin as described by Duskin et al., (1982) J. Biol. Chem. 257:3105-09. Tunicamycin blocks the formation of protein-N-glycoside linkages.

Hence, aspects of the present disclosure include glycosylation variants of antigen binding proteins that specifically bind to CHRDL-1 wherein the number and/or type of glycosylation site(s) has been altered compared to the amino acid sequences of the parent polypeptide. In certain embodiments, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X can be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other embodiments, one or more new N-linked sites are created. Antibodies typically have a N-linked glycosylation site in the Fc region.

### Labels and Effector Groups

In some embodiments, an antigen binding protein that specifically binds to CHRDL-1 comprises one or more labels. The term “labeling group” or “label” means any detectable label. Examples of suitable labeling groups include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent groups (e.g., FITC, rhodamine, lanthanide phosphors), enzymatic groups (e.g., horseradish peroxidase, β-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the labeling group is coupled to the antigen binding protein via spacer arms of various lengths to reduce potential steric hindrance. Various methods for labeling proteins are known in the art and can be used as is seen fit.

The term “effector group” means any group coupled to an antigen binding protein that specifically binds to CHRDL-1 and that acts as a cytotoxic agent. Examples for suitable effector groups are radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I). Other suitable groups include toxins, therapeutic groups, or chemotherapeutic groups. Examples of suitable groups include calicheamicin, auristatins, geldanamycin and cantansine. In some embodiments, the effector group is coupled to the antigen binding protein via spacer arms of various lengths to reduce potential steric hindrance.

In general, labels fall into a variety of classes, depending on the assay in which they are to be detected: a) isotopic labels, which can be radioactive or heavy isotopes; b) magnetic labels (e.g., magnetic particles); c) redox active moieties; d) optical dyes; enzymatic groups (e.g., horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.). In some embodiments, the labeling group is coupled to the antigen binding protein via spacer arms of various lengths to reduce potential steric hindrance. Various methods for labeling proteins are known in the art.

Specific labels include optical dyes, including, but not limited to, chromophores, phosphors and fluorophores, with the latter being specific in many instances. Fluorophores can be either “small molecule” fluores, or proteinaceous fluores.

By “fluorescent label” is meant any molecule that can be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 647, Alexa Fluor 660, Alexa Fluor 680), Cascade Blue, Cascade Yellow and R-phycoerythrin (PE) (Molecular Probes, Eugene, Oreg.), FITC, Rhodamine, and Texas Red (Pierce, Rockford, Ill.), Cy5, Cy5.5, Cy7 (Amersham Life Science, Pittsburgh, Pa.). Suitable optical dyes, including fluorophores, are described in Molecular Probes Handbook by Richard P. Haugland and in subsequent editions, including Molecular Probes Handbook, A Guide to Fluorescent Probes and Labeling Technologies, 11^{th} edition, Johnson and Spence (eds), hereby expressly incorporated by reference.

Suitable proteinaceous fluorescent labels also include, but are not limited to, green fluorescent protein, including a Renilla, Ptilosarcus, or Aequorea species of GFP (Chalfie et al., (1994) Science 263:802-805), eGFP (Clontech Labs., Inc., Genbank Accession Number U55762), blue fluorescent protein (BFP, Quantum Biotechnologies, Inc., Quebec, Canada; Stauber, (1998) Biotechniques 24:462-71; Heim et al., (1996) Curr. Biol. 6:178-82), enhanced yellow fluorescent protein (EYFP, Clontech Labs., Inc.), luciferase (Ichiki et al., (1993) J. Immunol. 150:5408-17), β-galactosidase (Nolan et al., (1988) Proc. Natl. Acad. Sci. U.S.A. 85:2603-07) and Renilla (WO92/15673, WO95/07463, WO98/14605, WO98/26277, WO99/49019, U.S. Pat. Nos. 5,292,658, 5,418,155, 5,683,888, 5,741,668, 5,777,079, 5,804,387, 5,874,304, 5,876,995 and 5,925,558).

### Preparation of Antigen Binding Proteins

Non-human antibodies that are provided can be, for example, derived from any antibody-producing animal, such as a mouse, rat, rabbit, goat, donkey, or non-human primate (such as a monkey, (e.g., cynomolgus or rhesus monkey) or an ape (e.g., chimpanzee)). Non-human antibodies can be used, for instance, in in vitro cell culture and cell-culture based applications, or any other application where an immune response to the antibody does not occur or is insignificant, can be prevented, is not a concern, or is desired.

In certain embodiments, the antibodies can be produced by immunizing with full length human CHRDL-1, as well as by other methods known in the art, e.g., as described in the Examples presented herein. The antibodies can be polyclonal, monoclonal, or can be synthesized in host cells by expressing recombinant DNA.

Fully human antibodies can be prepared as described above by immunizing transgenic animals containing human immunoglobulin loci or by selecting a phage display library that is expressing a repertoire of human antibodies.

The monoclonal antibodies (mAbs) can be produced by a variety of techniques, including conventional monoclonal antibody methodology, e.g., the standard somatic cell hybridization technique of Kohler & Milstein, (1975) Nature 256:495-97. Alternatively, other techniques for producing monoclonal antibodies can be employed, for example, the viral or oncogenic transformation of B-lymphocytes. One suitable animal system for preparing hybridomas is the murine system, which is a very well established procedure. Immunization protocols and techniques for isolation of immunized splenocytes for fusion are known in the art. For such procedures, B cells from immunized mice are fused with a suitable immortalized fusion partner, such as a murine myeloma cell line. If desired, rats or other mammals besides can be immunized instead of mice and B cells from such animals can be fused with the murine myeloma cell line to form hybridomas. Alternatively, a myeloma cell line from a source other than mouse can be used. Fusion procedures for making hybridomas also are well known. SLAM technology can also be employed in the production of antibodies.

Single chain antibodies that are provided can be formed by linking heavy and light chain variable domain (Fv region) fragments via an amino acid bridge (short peptide linker), resulting in a single polypeptide chain. Such single-chain Fvs (scFvs) can be prepared by fusing DNA encoding a peptide linker between DNAs encoding the two variable domain polypeptides (V_{L} and V_{H}). The resulting polypeptides can fold back on themselves to form antigen-binding monomers, or they can form multimers (e.g., dimers, trimers, or tetramers), depending on the length of a flexible linker between the two variable domains (Kortt et al., (1997) Prot. Eng. 10:423; Kortt et al., (2001) Biomol. Eng. 18:95-108). By combining different V_{L} and V_{H}-comprising polypeptides, one can form multimeric scFvs that bind to different epitopes (Kriangkum et al., (2001) Biomol. Eng. 18:31-40). Techniques developed for the production of single chain antibodies include those described in U.S. Pat. No. 4,946,778; Bird et al., (1988) Science 242:423-26; Huston et al., (1988) Proc. Natl. Acad. Sci. U.S.A. 85:5879-83; Ward et al., (1989) Nature 334:544-46, de Graaf et al., (2002) Methods Mol. Biol. 178:379-387. Single chain antibodies derived from antibodies provided herein include, but are not limited to scFvs comprising the variable domain combinations of the heavy and light chain variable regions depicted herein, or combinations of light and heavy chain variable domains which include the CDRs depicted in FIGS. 1A, 1B, 2, 7, and 8 herein.

Antibodies provided herein that are of one subclass can be changed to antibodies from a different subclass using subclass switching methods. Thus, IgG antibodies can be derived from an IgM antibody, for example, and vice versa. Such techniques allow the preparation of new antibodies that possess the antigen binding properties of a given antibody (the parent antibody), but also exhibit biological properties associated with an antibody isotype or subclass different from that of the parent antibody. Recombinant DNA techniques can be employed. Cloned DNA encoding particular antibody polypeptides can be employed in such procedures, e.g., DNA encoding the constant domain of an antibody of the desired isotype. See e.g., Lantto et al., (2002) Methods Mol. Biol. 178:303-16.

Accordingly, the antibodies that are provided include those comprising, for example, the variable domain combinations described, supra., having a desired isotype (for example, IgA, IgG1, IgG2, IgG3, IgG4, IgE, and IgD) as well as Fab or F(ab′)₂ fragments thereof. Moreover, if an IgG4 is desired, it can also be desired to introduce a point mutation (e.g., a mutation from CPSCP to CPPCP in the hinge region as described in Bloom et al., (1997) Protein Science 6:407-15, incorporated by reference herein) to alleviate a tendency to form intra-H chain disulfide bonds that can lead to heterogeneity in the IgG4 antibodies.

Moreover, techniques for deriving antibodies having different properties (i.e., varying affinities for the antigen to which they bind) are also known. One such technique, referred to as chain shuffling, involves displaying immunoglobulin variable domain gene repertoires on the surface of filamentous bacteriophage, often referred to as phage display. Chain shuffling has been used to prepare high affinity antibodies to the hapten 2-phenyloxazol-5-one, as described by Marks et al., (1992) Nature Biotechnology 10:779-83.

Conservative modifications can be made to the heavy and light chain variable regions described in Table 2, or the CDRs described in FIGS. 1A, 1B, 2, 7, and 8 (and corresponding modifications to the encoding nucleic acids) to produce an antigen binding protein having functional and biochemical characteristics. Methods for achieving such modifications are described herein.

Antigen binding proteins that specifically bind to CHRDL-1 can be further modified in various ways. For example, if they are to be used for therapeutic purposes, they can be conjugated with polyethylene glycol (PEGylated) to prolong the serum half-life or to enhance protein delivery. PEG can be attached directly to the antigen binding protein or it can be attached via a linker, such as a glycosidic linkage.

Alternatively, the V region of the subject antibodies or fragments thereof can be fused with the Fc region of a different antibody molecule. The Fc region used for this purpose can be modified so that it does not bind complement, thus reducing the likelihood of inducing cell lysis in the patient when the fusion protein is used as a therapeutic agent. In addition, the subject antibodies or functional fragments thereof can be conjugated with human serum albumin to enhance the serum half-life of the antibody or fragment thereof. Another useful fusion partner for the antigen binding proteins or fragments thereof is transthyretin (TTR). TTR has the capacity to form a tetramer, thus an antibody-TTR fusion protein can form a multivalent antibody which can increase its binding avidity.

Alternatively, substantial modifications in the functional and/or biochemical characteristics of the antigen binding proteins described herein can be achieved by creating substitutions in the amino acid sequence of the heavy and light chains that differ significantly in their effect on maintaining (a) the structure of the molecular backbone in the area of the substitution, for example, as a sheet or helical conformation, (b) the charge or hydrophobicity of the molecule at the target site, or (c) the bulkiness of the side chain. A “conservative amino acid substitution” can involve a substitution of a native amino acid residue with a normative residue that has little or no effect on the polarity or charge of the amino acid residue at that position as described supra. Furthermore, any native residue in the polypeptide can also be substituted with alanine, as has been previously described for alanine scanning mutagenesis.

Amino acid substitutions (whether conservative or non-conservative) of the subject antibodies can be implemented by those skilled in the art by applying routine techniques. Amino acid substitutions can be used to identify important residues of the antibodies provided herein, or to increase or decrease the affinity of these antibodies to CHRDL-1 or for modifying the binding affinity of other antigen-binding proteins described herein.

### Methods of Expressing Antigen Binding Proteins

Expression systems and constructs in the form of plasmids, expression vectors, transcription or expression cassettes that comprise at least one polynucleotide as described above are also provided herein, as well host cells comprising such expression systems or constructs.

The antigen binding proteins provided herein can be prepared by any of a number of conventional techniques. For example, antigen binding proteins that specifically bind to CHRDL-1 can be produced by recombinant expression systems, using any technique known in the art. See e.g., Monoclonal Antibodies, Hybridomas: A New Dimension in Biological Analyses, (Kennet et al., eds.) Plenum Press (1980) and subsequent editions; and Harlow & Lane, (1988) supra.

Antigen binding proteins can be expressed in hybridoma cell lines (e.g., in particular antibodies can be expressed in hybridomas) or in cell lines other than hybridomas. Expression constructs encoding the antibodies can be used to transform a mammalian, insect or microbial host cell. Transformation can be performed using any known method for introducing polynucleotides into a host cell, including, for example packaging the polynucleotide in a virus or bacteriophage and transducing a host cell with the construct by transfection procedures known in the art, as exemplified by U.S. Pat. Nos. 4,399,216; 4,912,040; 4,740,461; and 4,959,455. The optimal transformation procedure used will depend upon which type of host cell is being transformed. Methods for introduction of heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, mixing nucleic acid with positively-charged lipids, and direct microinjection of the DNA into nuclei.

Recombinant expression constructs typically comprise a nucleic acid molecule encoding a polypeptide comprising one or more of the following: one or more CDRs provided herein; a light chain constant region; a light chain variable region; a heavy chain constant region (e.g., C_{H}1, C_{H}2 and/or C_{H}3); and/or another scaffold portion of an antigen binding protein. These nucleic acid sequences are inserted into an appropriate expression vector using standard ligation techniques. The vector is typically selected to be functional in the particular host cell employed (i.e., the vector is compatible with the host cell machinery, permitting amplification and/or expression of the gene can occur). In some embodiments, vectors are used that employ protein-fragment complementation assays using protein reporters, such as dihydrofolate reductase (see, e.g., U.S. Pat. No. 6,270,964, which is hereby incorporated by reference). Suitable expression vectors can be purchased, for example, from Invitrogen Life Technologies or BD Biosciences. Other useful vectors for cloning and expressing the antibodies and fragments include those described in Bianchi and McGrew, (2003) Biotech. Biotechnol. Bioeng. 84:439-44, which is hereby incorporated by reference. Additional suitable expression vectors are discussed, for example, in “Gene Expression Technology,” Methods Enzymol., vol. 185, (Goeddel et al., ed.), (1990), Academic Press.

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element.

Optionally, an expression vector can contain a “tag”-encoding sequence, i.e., an oligonucleotide molecule located at the 5′ or 3′ end of an antigen binding protein coding sequence; the oligonucleotide sequence encodes polyHis (such as hexaHis, HHHHHH), or another “tag” such as FLAG, HA (hemaglutinin influenza virus), or myc, for which commercially available antibodies exist. This tag is typically fused to the polypeptide upon expression of the polypeptide, and can serve as a means for affinity purification or detection of the antigen binding protein from the host cell. Affinity purification can be accomplished, for example, by column chromatography using antibodies against the tag as an affinity matrix. Optionally, the tag can subsequently be removed from the purified antigen binding protein by various means such as using certain peptidases for cleavage.

Flanking sequences can be homologous (i.e., from the same species and/or strain as the host cell), heterologous (i.e., from a species other than the host cell species or strain), hybrid (i.e., a combination of flanking sequences from more than one source), synthetic or native. As such, the source of a flanking sequence can be any prokaryotic or eukaryotic organism, any vertebrate or invertebrate organism, or any plant, provided that the flanking sequence is functional in, and can be activated by, the host cell machinery.

Flanking sequences useful in the vectors can be obtained by any of several methods well known in the art. Typically, flanking sequences useful herein will have been previously identified by mapping and/or by restriction endonuclease digestion and can thus be isolated from the proper tissue source using the appropriate restriction endonucleases. In some cases, the full nucleotide sequence of a flanking sequence can be known. Here, the flanking sequence can be synthesized using the methods described herein for nucleic acid synthesis or cloning.

Whether all or only a portion of the flanking sequence is known, it can be obtained using polymerase chain reaction (PCR) and/or by screening a genomic library with a suitable probe such as an oligonucleotide and/or flanking sequence fragment from the same or another species. Where the flanking sequence is not known, a fragment of DNA containing a flanking sequence can be isolated from a larger piece of DNA that can contain, for example, a coding sequence or even another gene or genes. Isolation can be accomplished by restriction endonuclease digestion to produce the proper DNA fragment followed by isolation using agarose gel purification, column chromatography or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. If the vector of choice does not contain an origin of replication site, one can be chemically synthesized based on a known sequence, and ligated into the vector. For example, the origin of replication from the plasmid pBR322 (GenBank Accession #J01749, New England Biolabs, Beverly, Mass.) is suitable for most gram-negative bacteria, and various viral origins (e.g., SV40, polyoma, adenovirus, vesicular stomatitus virus (VSV), or papillomaviruses such as HPV or BPV) are useful for cloning vectors in mammalian cells. Generally, the origin of replication component is not needed for mammalian expression vectors (for example, the SV40 origin is often used only because it also contains the virus early promoter).

A transcription termination sequence is typically located 3′ to the end of a polypeptide coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using methods for nucleic acid synthesis such as those described herein.

A selectable marker gene encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex or defined media. Specific selectable markers are the kanamycin resistance gene, the ampicillin resistance gene, and the tetracycline resistance gene. Advantageously, a neomycin resistance gene can also be used for selection in both prokaryotic and eukaryotic host cells.

Other selectable genes can be used to amplify the gene that will be expressed. Amplification is the process wherein genes that are required for production of a protein critical for growth or cell survival are reiterated in tandem within the chromosomes of successive generations of recombinant cells. Examples of suitable selectable markers for mammalian cells include dihydrofolate reductase (DHFR) and promoterless thymidine kinase genes. Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to the amplification of both the selectable gene and the DNA that encodes another gene, such as an antigen binding protein that binds to CHRDL-1. As a result, increased quantities of a polypeptide such as an antigen binding protein are synthesized from the amplified DNA.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed.

In some cases, such as where glycosylation is desired in a eukaryotic host cell expression system, one can manipulate the various pre- or pro-sequences to improve glycosylation or yield. For example, one can alter the peptidase cleavage site of a particular signal peptide, or add prosequences, which also can affect glycosylation. The final protein product can have, in the −1 position (relative to the first amino acid of the mature protein), one or more additional amino acids incident to expression, which may not have been totally removed. For example, the final protein product can have one or two amino acid residues found in the peptidase cleavage site, attached to the amino-terminus. Alternatively, use of some enzyme cleavage sites can result in a slightly truncated form of the desired polypeptide, if the enzyme cuts at such area within the mature polypeptide.

Expression and cloning will typically contain a promoter that is recognized by the host organism and operably linked to the molecule encoding an antigen binding protein that specifically binds to CHRDL-1. Promoters are untranscribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, uniformly transcribe a gene to which they are operably linked, that is, with little or no control over gene expression. A large number of promoters, recognized by a variety of potential host cells, are well known. A suitable promoter is operably linked to the DNA encoding heavy chain or light chain comprising an antigen binding protein by removing the promoter from the source DNA by restriction enzyme digestion and inserting the desired promoter sequence into the vector.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus, and Simian Virus 40 (SV40). Other suitable mammalian promoters include heterologous mammalian promoters, for example, heat-shock promoters and the actin promoter.

Additional promoters which can be of interest include, but are not limited to: SV40 early promoter (Benoist & Chambon, (1981) Nature 290:304-310); CMV promoter (Thomsen et al., (1984) Proc. Natl. Acad. U.S.A. 81:659-663); the promoter contained in the 3′ long terminal repeat of Rous sarcoma virus (Yamamoto et al., (1980) Cell 22:787-97); herpes thymidine kinase promoter (Wagner et al., (1981) Proc. Natl. Acad. Sci. U.S.A. 78:1444-45); promoter and regulatory sequences from the metallothionine gene (Prinster et al., (1982) Nature 296:39-42); and prokaryotic promoters such as the beta-lactamase promoter (Villa-Kamaroff et al., (1978) Proc. Natl. Acad. Sci. U.S.A. 75:3727-31); or the tac promoter (DeBoer et al., (1983) Proc. Natl. Acad. Sci. U.S.A. 80:21-25). Also of interest are the following animal transcriptional control regions, which exhibit tissue specificity and have been utilized in transgenic animals: the elastase I gene control region that is active in pancreatic acinar cells (Swift et al., (1984) Cell 38:639-46; Ornitz et al., (1986) Cold Spring Harbor Symp. Quant. Biol. 50:399-409; MacDonald, (1987) Hepatology 7:425-515); the insulin gene control region that is active in pancreatic beta cells (Hanahan, (1985) Nature 315:115-22); the immunoglobulin gene control region that is active in lymphoid cells (Grosschedl et al., (1984) Cell 38:647-58; Adames et al., (1985) Nature 318:533-38; Alexander et al., (1987) Mol. Cell. Biol. 7:1436-44); the mouse mammary tumor virus control region that is active in testicular, breast, lymphoid and mast cells (Leder et al., (1986) Cell 45:485-95); the albumin gene control region that is active in liver (Pinkert et al., (1987) Genes and Devel. 1:268-76); the alpha-feto-protein gene control region that is active in liver (Krumlauf et al., (1985) Mol. Cell. Biol. 5:1639-48; Hammer et al., (1987) Science 253:53-58); the alpha 1-antitrypsin gene control region that is active in liver (Kelsey et al., (1987) Genes and Devel. 1:161-71); the beta-globin gene control region that is active in myeloid cells (Mogram et al., (1985) Nature 315:338-40; Kollias et al., (1986) Cell 46:89-94); the myelin basic protein gene control region that is active in oligodendrocyte cells in the brain (Readhead et al., (1987) Cell 48:703-12); the myosin light chain-2 gene control region that is active in skeletal muscle (Sani, (1985) Nature 314:283-86); and the gonadotropic releasing hormone gene control region that is active in the hypothalamus (Mason et al., (1986) Science 234:1372-78).

An enhancer sequence can be inserted into the vector to increase transcription of DNA encoding light chain or heavy chain comprising an antigen binding protein that specifically binds to CHRDL-1 by higher eukaryotes, e.g., a human antigen binding protein that specifically binds to CHRDL-1. Enhancers are cis-acting elements of DNA, usually about 10-300 by in length, that act on the promoter to increase transcription. Enhancers are relatively orientation and position independent, having been found at positions both 5′ and 3′ to the transcription unit. Several enhancer sequences available from mammalian genes are known (e.g., globin, elastase, albumin, alpha-feto-protein and insulin). Typically, however, an enhancer from a virus is used. The SV40 enhancer, the cytomegalovirus early promoter enhancer, the polyoma enhancer, and adenovirus enhancers known in the art are exemplary enhancing elements for the activation of eukaryotic promoters. While an enhancer can be positioned in the vector either 5′ or 3′ to a coding sequence, it is typically located at a site 5′ from the promoter. A sequence encoding an appropriate native or heterologous signal sequence (leader sequence or signal peptide) can be incorporated into an expression vector, to promote extracellular secretion of the antibody. The choice of signal peptide or leader depends on the type of host cells in which the antibody is to be produced, and a heterologous signal sequence can replace the native signal sequence. Examples of signal peptides that are functional in mammalian host cells include the following: the signal sequence for interleukin-7 (IL-7) described in U.S. Pat. No. 4,965,195; the signal sequence for interleukin-2 receptor described in Cosman et al., (1984) Nature 312:768-71; the interleukin-4 receptor signal peptide described in EP Patent No. 0367 566; the type I interleukin-1 receptor signal peptide described in U.S. Pat. No. 4,968,607; the type II interleukin-1 receptor signal peptide described in EP Patent No. 0 460 846.

Expression vectors can be constructed from a starting vector such as a commercially available vector. Such vectors can but need not contain all of the desired flanking sequences. Where one or more of the flanking sequences are not already present in the vector, they can be individually obtained and ligated into the vector. Methods used for obtaining each of the flanking sequences are well known to one skilled in the art.

After the vector has been constructed and a nucleic acid molecule encoding light chain, a heavy chain, or a light chain and a heavy chain comprising an antigen binding protein that specifically binds to CHRDL-1 has been inserted into the proper site of the vector, the completed vector can be inserted into a suitable host cell for amplification and/or polypeptide expression. The transformation of an expression vector for an antigen binding protein into a selected host cell can be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook et al., (2001), supra.

A host cell, when cultured under appropriate conditions, synthesizes an antigen binding protein that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Mammalian cell lines available as hosts for expression are well known in the art and include, but are not limited to, immortalized cell lines available from the American Type Culture Collection (ATCC), including but not limited to HeLa cells, Human Embryonic Kidney 293 cells (HEK293 cells), Chinese hamster ovary (CHO) cells, HeLa cells, baby hamster kidney (BHK) cells, monkey kidney cells (COS), human hepatocellular carcinoma cells (e.g., Hep G2), and a number of other cell lines. In certain embodiments, cell lines can be selected through determining which cell lines have high expression levels and constitutively produce antigen binding proteins with desirable binding properties (e.g., the ability to bind to CHRDL-1. In another embodiment, a cell line from the B cell lineage that does not make its own antibody but has a capacity to make and secrete a heterologous antibody can be selected.

### Uses of Antigen Binding Proteins for Diagnostic and Therapeutic Purposes

The antigen binding proteins disclosed herein are useful for detecting CHRDL-1 in biological samples and identification of cells or tissues that produce CHRDL-1. For instance, the antigen binding proteins disclosed herein can be used in diagnostic assays, e.g., binding assays to detect and/or quantify CHRDL-1 expressed in a tissue or cell.

Antigen binding proteins that specifically bind to CHRDL-1 can also be used in treatment of diseases related to CHRDL-1 activity in a patient in need thereof, such as diabetes, obesity, dyslipidemia, NASH, cardiovascular disease, and metabolic syndrome.

### Indications

A disease or condition associated with human CHRDL-1 includes any disease or condition whose onset in a patient is influenced by, at least in part, undesired levels of CHRDL-1. Examples of diseases and conditions that can be treated with the antigen binding proteins provided herein include type 2 diabetes, obesity, dyslipidemia, NASH, cardiovascular disease, and metabolic syndrome. The antigen binding proteins described herein can be employed as a prophylactic treatment administered, e.g., daily, weekly, biweekly, monthly, bimonthly, biannually, etc. to prevent or reduce the frequency and/or severity of symptoms, e.g., elevated plasma glucose levels, elevated triglycerides and/or cholesterol levels, thereby providing an improved glycemic and cardiovascular risk factor profile.

### Diagnostic Methods

The antigen binding proteins described herein can be used for diagnostic purposes to detect, diagnose, or monitor diseases and/or conditions associated with CHRDL-1. Also provided are methods for the detection of the presence of CHRDL-1 in a sample using classical immunohistological methods known to those of skill in the art (e.g., Tijssen, (1985) “Practice and Theory of Enzyme Immunoassays” in Laboratory Techniques in Biochemistry and Molecular Biology, 15 (Burdon & van Knippenberg, eds.), Elsevier Biomedical); Zola, (1987) Monoclonal Antibodies: A Manual of Techniques, pp. 147-58 (CRC Press, Inc.); Jalkanen et al., (1985) J. Cell. Biol. 101:976-85; Jalkanen et al., (1987) J. Cell Biol. 105:3087-96). The detection of CHRDL-1 can be performed in vivo or in vitro.

Examples of methods useful in the detection of the presence of CHRDL-1 include immunoassays, such as the enzyme linked immunosorbent assay (ELISA) and the radioimmunoassay (RIA).

For diagnostic applications, the antigen binding protein typically will be labeled with a detectable labeling group. Suitable labeling groups include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent groups (e.g., FITC, rhodamine, lanthanide phosphors), enzymatic groups (e.g., horseradish peroxidase, β-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the labeling group is coupled to the antigen binding protein via spacer arms of various lengths to reduce potential steric hindrance. Various methods for labeling proteins are known in the art and can be used.

In another aspect, an antigen binding protein can be used to identify a cell or cells that express CHRDL-1. In a specific embodiment, the antigen binding protein is labeled with a labeling group and the binding of the labeled antigen binding protein to CHRDL-1 is detected. In a further specific embodiment, the antigen binding protein is isolated and measured using techniques known in the art. See, for example, Harlow & Lane, (1988) supra; Current Protocols In Immunology (John E. Coligan, ed), John Wiley & Sons (1993 ed., and supplements and/or updates).

Another aspect provides for detecting the presence of a test molecule that competes for binding to CHRDL-1 with the antigen binding proteins provided herein. An example of one such assay could involve detecting the amount of free antigen binding protein in a solution containing an amount of CHRDL-1 in the presence or absence of the test molecule. An increase in the amount of free antigen binding protein (i.e., the antigen binding protein not bound to CHRDL-1 would indicate that the test molecule is capable of competing for binding to CHRDL-1 with the antigen binding protein. In one embodiment, the antigen binding protein is labeled with a labeling group. Alternatively, the test molecule is labeled and the amount of free test molecule is monitored in the presence and absence of an antigen binding protein.

### Methods of Treatment: Pharmaceutical Formulations and Routes of Administration

Methods of using the disclosed antigen binding proteins are also provided. In some methods, an antigen binding protein is provided to a patient, which inhibits CHRDL-1 activity.

Pharmaceutical compositions that comprise a therapeutically effective amount of one or a plurality of the antigen binding proteins and a pharmaceutically acceptable diluent, carrier, solubilizer, emulsifier, preservative, and/or adjuvant are also provided. In addition, methods of treating a patient by administering such pharmaceutical composition are included. The term “patient” includes human patients.

Acceptable formulation materials are nontoxic to recipients at the dosages and concentrations employed. In specific embodiments, pharmaceutical compositions comprising a therapeutically effective amount of human antigen binding proteins that specifically bind to CHRDL-1 are provided.

In certain embodiments, acceptable formulation materials preferably are nontoxic to recipients at the dosages and concentrations employed. In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as Pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See e.g., Remington's Pharmaceutical Sciences, 18th Edition, (A. R. Gennaro, ed.), 1990, Mack Publishing Company, and subsequent editions.

In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions can influence the physical state, stability, rate of in vivo release and rate of in vivo clearance of the antigen binding proteins disclosed. In certain embodiments, the primary vehicle or carrier in a pharmaceutical composition can be either aqueous or non-aqueous in nature. For example, a suitable vehicle or carrier can be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and can further include sorbitol or a suitable substitute. In certain embodiments, compositions comprising antigen binding proteins that specifically bind to CHRDL-1 can be prepared for storage by mixing the selected composition having the desired degree of purity with optional formulation agents (see, Remington's Pharmaceutical Sciences, supra for examples of suitable formulation agents) in the form of a lyophilized cake or an aqueous solution. Further, in certain embodiments, antigen binding proteins that bind to CHRDL-1 can be formulated as a lyophilizate using appropriate excipients such as sucrose. The pharmaceutical compositions can also be selected for parenteral delivery. Alternatively, the compositions can be selected for inhalation or for delivery through the digestive tract, such as orally. Preparation of such pharmaceutically acceptable compositions is within the skill of the art.

The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the therapeutic compositions can be provided in the form of a pyrogen-free, parenterally acceptable aqueous solution comprising the desired antigen binding protein in a pharmaceutically acceptable vehicle. A particularly suitable vehicle for parenteral injection is sterile distilled water in which the antigen binding protein is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide controlled or sustained release of the product which can be delivered via depot injection. In certain embodiments, hyaluronic acid can also be used, which can have the effect of promoting sustained duration in the circulation. In certain embodiments, implantable drug delivery devices can be used to introduce the desired antigen binding protein.

Certain pharmaceutical compositions are formulated for inhalation. In some embodiments, antigen binding proteins that bind to CHRDL-1 are formulated as a dry, inhalable powder. In specific embodiments, antigen binding protein inhalation solutions can also be formulated with a propellant for aerosol delivery. In certain embodiments, solutions can be nebulized. Pulmonary administration and formulation methods therefore are further described in International Patent Application No. PCT/US94/001875, which is incorporated by reference and describes pulmonary delivery of chemically modified proteins. Some formulations can be administered orally. Antigen binding proteins that specifically bind to CHRDL-1 that are administered in this fashion can be formulated with or without carriers customarily used in the compounding of solid dosage forms such as tablets and capsules. In certain embodiments, a capsule can be designed to release the active portion of the formulation at the point in the gastrointestinal tract when bioavailability is maximized and pre-systemic degradation is minimized. Additional agents can be included to facilitate absorption of an antigen binding protein. Diluents, flavorings, low melting point waxes, vegetable oils, lubricants, suspending agents, tablet disintegrating agents, and binders can also be employed.

Some pharmaceutical compositions comprise an effective quantity of one or a plurality of human antigen binding proteins that specifically bind to CHRDL-1 in a mixture with non-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving antigen binding proteins that specifically bind to CHRDL-1 in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which is incorporated by reference and describes controlled release of porous polymeric microparticles for delivery of pharmaceutical compositions. Sustained-release preparations can include semipermeable polymer matrices in the form of shaped articles, e.g., films, or microcapsules. Sustained release matrices can include polyesters, hydrogels, polylactides (as disclosed in U.S. Pat. No. 3,773,919 and European Patent Application Publication No. EP 058481, each of which is incorporated by reference), copolymers of L-glutamic acid and gamma ethyl-L-glutamate (Sidman et al., (1983) Biopolymers 2:547-556), poly(2-hydroxyethyl-inethacrylate) (Langer et al., (1981) J. Biomed. Mater. Res. 15:167-277 and Langer, (1982) Chem. Tech. 12:98-105), ethylene vinyl acetate (Langer et al., (1981) supra) or poly-D(−)-3-hydroxybutyric acid (European Patent Application Publication No. EP 133988). Sustained release compositions can also include liposomes that can be prepared by any of several methods known in the art. See e.g., Eppstein et al., (1985) Proc. Natl. Acad. Sci. U.S.A. 82:3688-3692; European Patent Application Publication Nos. EP 036676; EP 088046 and EP 143949, incorporated by reference.

Pharmaceutical compositions used for in vivo administration are typically provided as sterile preparations. Sterilization can be accomplished by filtration through sterile filtration membranes. When the composition is lyophilized, sterilization using this method can be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

In certain embodiments, cells expressing a recombinant antigen binding protein as disclosed herein are encapsulated for delivery (see, Tao et al., Invest. Ophthalmol. Vis. Sci. (2002) 43:3292-3298 and Sieving et al., PNAS USA (2006) 103:3896-3901).

In certain formulations, an antigen binding protein has a concentration of between 10 mg/ml and 150 mg/ml. Some formulations contain a buffer, sucrose and polysorbate. An example of a formulation is one containing 50-100 mg/ml of antigen binding protein, 5-20 mM sodium acetate, 5-10% w/v sucrose, and 0.002-0.008% w/v polysorbate. Certain, formulations, for instance, contain 1-100 mg/ml of an antigen binding protein in 9-11 mM sodium acetate buffer, 8-10% w/v sucrose, and 0.005-0.006% w/v polysorbate. The pH of certain such formulations is in the range of 4.5-6. Other formulations can have a pH of 5.0-5.5.

Once the pharmaceutical composition has been formulated, it can be stored in sterile vials as a solution, suspension, gel, emulsion, solid, crystal, or as a dehydrated or lyophilized powder. Such formulations can be stored either in a ready-to-use form or in a form (e.g., lyophilized) that is reconstituted prior to administration. Kits for producing a single-dose administration unit are also provided. Certain kits contain a first container having a dried protein and a second container having an aqueous formulation. In certain embodiments, kits containing single and multi-chambered pre-filled syringes (e.g., liquid syringes and lyosyringes) are provided. The therapeutically effective amount of an antigen binding protein-containing pharmaceutical composition to be employed will depend, for example, upon the therapeutic context and objectives. One skilled in the art will appreciate that the appropriate dosage levels for treatment will vary depending, in part, upon the molecule delivered, the indication for which the antigen binding protein is being used, the route of administration, and the size (body weight, body surface or organ size) and/or condition (the age and general health) of the patient. In certain embodiments, the clinician can titer the dosage and modify the route of administration to obtain the optimal therapeutic effect.

A typical dosage can range from about 1 mg/kg to up to about 30 mg/kg or more, depending on the factors mentioned above. In specific embodiments, the dosage can range from 10 μg/kg up to about 35 mg/kg, optionally from 0.1 mg/kg up to about 35 mg/kg, alternatively from 0.3 mg/kg up to about 20 mg/kg. In some applications, the dosage is from 0.5 mg/kg to 20 mg/kg and in other applications the dosage is from 21-100 mg/kg. In some instances, an antigen binding protein is dosed at 0.3-20 mg/kg. The dosage schedule in some treatment regimes is at a dose of 0.3 mg/kg qW-20 mg/kg qW.

Dosing frequency will depend upon the pharmacokinetic parameters of the particular antigen binding protein in the formulation used. Typically, a clinician administers the composition until a dosage is reached that achieves the desired effect. The composition can therefore be administered as a single dose, or as two or more doses (which can but need not contain the same amount of the desired molecule) over time, or as a continuous infusion via an implantation device or catheter. Appropriate dosages can be ascertained through use of appropriate dose-response data. In certain embodiments, the antigen binding proteins can be administered to patients throughout an extended time period. Chronic administration of an antigen binding protein minimizes the adverse immune or allergic response commonly associated with antigen binding proteins that are not fully human, for example an antibody raised against a human antigen in a non-human animal, for example, a non-fully human antibody or non-human antibody produced in a non-human species.

The route of administration of the pharmaceutical composition is in accord with known methods, e.g., orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions can be administered by bolus injection or continuously by infusion, or by implantation device.

The composition also can be administered locally via implantation of a membrane, sponge or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration.

It also can be desirable to use antigen binding protein pharmaceutical compositions ex vivo. In such instances, cells, tissues or organs that have been removed from the patient are exposed to antigen binding protein pharmaceutical compositions after which the cells, tissues and/or organs are subsequently implanted back into the patient.

In particular, antigen binding proteins that specifically bind to CHRDL-1 can be delivered by implanting certain cells that have been genetically engineered, using methods such as those described herein and known in the art, to express and secrete the polypeptide. In certain embodiments, such cells can be animal or human cells, and can be autologous, heterologous, or xenogeneic. In certain embodiments, the cells can be immortalized. In other embodiments, in order to decrease the chance of an immunological response, the cells can be encapsulated to avoid infiltration of surrounding tissues. In further embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

### Combination Therapies

In another aspect, the present disclosure provides a method of treating a subject for diabetes with a therapeutic antigen binding protein of the present disclosure, such as the therapeutic antibodies described herein, together with one or more other treatments. In one embodiment, such a combination therapy achieves an additive or synergistic effect. The antigen binding proteins can be administered in combination with one or more of the type 2 diabetes or obesity treatments currently available. These treatments for diabetes include biguanide (metaformin), and sulfonylureas (such as glyburide, glipizide). Additional treatments directed at maintaining glucose homeostasis include PPAR gamma agonists (pioglitazone, rosiglitazone); glinides (meglitinide, repaglinide, and nateglinide); DPP-4 inhibitors (Januvia® and Onglyza®) and alpha glucosidase inhibitors (acarbose, voglibose). Additional combination treatments for diabetes include injectable treatments such as insulin and incretin mimetics (Byetta®, Exenatide®), other GLP-1 (glucagon-like peptide) analogs such as Victoza® (liraglutide), other GLP-1R agonists and Symlin® (pramlintide). Additional combination treatments directed at weight loss include Meridia® and Xenical®.

The following examples, including the experiments conducted and the results achieved, are provided for illustrative purposes only and are not to be construed as limiting.

### Example 1

### Recombinant Expression of CHRDL-1

Recombinant human CHRDL-1 is commercially available from R&D Systems (2013 cat#1808-NR). It is described as Glu22-Cys450 of the amino acid sequence set forth in SEQ ID NO: 1 with a C-terminal 10xHis tag and is expressed in mouse myeloma NSO cells. Alternatively, recombinant CHRDL-1 can be produced in other mammalian cells (e.g., CHO).

Murine CHRDL-1 Expression

Murine chordin-like 1 was stably expressed in CHO-S cells (Invitrogen™) using an appropriate mammalian expression vector (e.g. CMV enhancer driven expression). The mammalian expression vector used was designed to express the amino acid sequence in SEQ ID NO: 3 with a C-terminal DEVD-6xHis tag. Transfections were performed using Lipofectamine LTX (Invitrogen) according to the manufacturer's protocol. Briefly, 4 μg of the mammalian expression plasmid DNA for expression of muChrdl1-DEVD-6xHis was added to 0.5 ml OPTI-MEM (Gibco) and mixed. In a separate tube, 10 μl Lipofectamine LTX was added to 0.5 ml OPTI-MEM. The solutions were incubated for 5 minutes at room temperature. To form the transfection complex, the DNA and Lipofectamine LTX mixtures were combined and incubated at room temperature for an additional 20 minutes.

Log phase CHO-S cells were pelleted by centrifugation (1000 RPM for 5 minutes), washed one time with 1×PBS (Gibco), and resuspended to 1e6 viable cells/mL in OPTI-MEM. 1 mL of the washed cells was added to the well of a 6 well plate. The DNA transfection complex was added to the cells. The plate was incubated at 36° C., 5% CO₂, shaking at 115 RPM for 6 hours. To stop the transfection, 2 ml growth media was added to the well and incubated for 48 to 72 hours.

To begin selection, cells were pellet by centrifugation (1000 RPM for 5 minutes) and the condition media was replaced with 4 mL to 6 mL of growth media supplemented with 10 μg/mL puromycin (Sigma). Selection media was changed in this fashion 2-3 times per week until cell viability and density recovered.

Productions were performed at small and large scale (up to 25 Liters) in shake flasks or the WAVE platforms (GE Healthcare Biosciences). Cells were seeded at 1e6vc/mL in production media and expression was generally carried out at 34° C. for 4-7 days. Cells were cleared by centrifugation and conditioned media was collected after filtration.

Human CHRDL-1 Expression

Human CHRDL-1 was produced in CHO-S cells (Invitrogen™) generally as described above for murine CHRDL-1. The mammalian expression vector used was designed to express Glu22-Cys450 of the amino acid sequence set forth in SEQ ID NO: 1 with a C-terminal DEVD-6xHis tag.

### Example 2

### Purification of Recombinant muCHRDL-1-DEVD-6xHis

Recombinant muCHRDL1-DEVD-6xHis was purified from mammalian host cells as described below. All purification processes were carried out at 4° C.; and the purification scheme used metal affinity chromatography followed by cation exchange chromatography.

Metal Affinity Chromatography

The mammalian host cell conditioned medium (CM) was centrifuged in a Beckman J6-M1 centrifuge at 4000 rpm for 1 hour at 4° C. to remove cell debris. The CM supernatant was then filtered through a sterile 0.2 μm cellulose acetate filter. At this point the sterile filtered CM was stored frozen until the purification commenced. The CM was thawed first at room temperature, followed by warm water and finally at 4° C. Following thawing the CM was filtered through a sterile 0.2 μm cellulose acetate filter and concentrated and buffer exchanged by tangential flow ultrafiltration (TFF) using a 10 kDa molecular weight cut-off membrane. The CM concentrate was diafiltered against 20 mM sodium phosphate, 0.5 M NaCl, 20 mM imidazole, pH 7.4. The UF/DF material was then loaded onto a Ni-NTA Superflow column (Qiagen) equilibrated in 20 mM sodium phosphate, 0.5 M NaCl, 20 mM imidazole, pH 7.4.

After loading, the Ni-NTA column was washed with 20 mM sodium phosphate, 0.5 M NaCl, 20 mM imidazole, pH 7.4 with 3 column volumes or until the absorbance at 280 nm of the flow-through returned to pre-load baseline. The muChrdl1-DEVD-6xHis was then eluted from the column using a linear gradient from 20 mM to 330 mM imidazole in 20 mM sodium phosphate, 0.5 M NaCl, pH 7.4. The absorbance at 280 nm of the eluate was monitored and fractions containing protein were collected. The fractions were then assayed by Coomassie-stained SDS-PAGE and by anti-polyhistidine Western blot to identify fractions containing a polypeptide that migrated at the expected size of murine CHRDL-1 with the C-terminal DEVD-6xHis tag. The appropriate fractions from the column were combined to make the Ni-NTA pool.

Cation Exchange Chromatography

The muCHRDL-1-DEVD-6xHis elution pool from the Ni-NTA column was further purified by cation exchange chromatography using SP High Performance (SPHP) chromatography media (GE Healthcare). The Ni-NTA pool was buffer exchanged into 20 mM NaOAc, pH 5.0 by dialysis using 10,000 MWCO membranes (Pierce Slide-A-Lyzer). The dialyzed Ni-NTA pool was then loaded onto an SPHP column equilibrated in 20 mM NaOAc, 50 mM NaCl, pH 5.0. After loading, the column was washed with 20 mM NaOAc, 50 mM NaCl, pH 5.0 with 3 column volumes or until the absorbance at 280 nm of the flow-through returned to baseline. The muCHRDL-1-DEVD-6xHis was then eluted from the SPHP column using a linear gradient from 50 mM to 500 mM sodium chloride in 20 mM NaOAc, pH 5.0. The absorbance at 280 nm of the eluate was monitored and the eluted muCHRDL-1-DEVD-6xHis was collected in fractions. The fractions were then assayed by Coomassie-stained SDS-PAGE to identify fractions containing a polypeptide that migrated at the expected size of muChrdl1-DEVD-6xHis. The appropriate fractions were combined to make the SPHP pool.

Formulation

Following purification, the SPHP pool was formulated in 10 mM NaOAc, 150 mM NaCl, pH 5.0 by dialysis using 10,000 MWCO membranes (Pierce Slide-A-Lyzer). Following formulation, the muCHRDL-1-DEVD-6xHis was filtered through a sterile 0.2 μm cellulose acetate filter and stored in aliquots at −70° C.

### Example 3

### Cell Based Assay for Identifying Agents Capable of Inhibiting CHRDL-1 Activity

CHRDL-1 has been shown to inhibit bone morphogenetic protein (BMP) signaling activity (Chandra et al., Biochem. Biophys. Res. Commun. 344:786-791 (2006); Larman et al., J. Am. Soc. Nephrol., 20:1020-1031 (2009); Fernandes et al., Cells Tissue Organs_{—}191:443-452 (2010). Various cell lines that are responsive to recombinant BMPs have been described (e.g. MC3T3-E1, C2C12) and this activity can be assessed by measuring endogenous markers (e.g., alkaline phosphatase) or by measuring transfected/transduced reporter gene expression (e.g., BRE-luciferase) representing BMP signaling activity (Korchynskyi et al., J. Biol. Chem. 277:4883-4891 (2002). Appropriate cell lines and assays can thus be used to measure CHRDL-1 antagonistic activity and further to identify CHRDL-1 antibodies that neutralize the antagonistic activity of CHRDL-1.

### Identification of CHRDL-1 Neutralizing Antibodies

MC3T3-E1 osteoblast-lineage cells that had been stably transduced (lentivirus) with a BMP signaling reporter gene containing a BMP Responsive Element (BRE) driving transcription of a luciferase gene were used for screening. The lentiviral construct also contained a neo expression cassette for positive selection purposes (e.g., G418; Geneticin). Human BMP4 (R&D Systems, 2013 cat#314-BP) was used to stimulate BMP signaling in these MC3T3-E1-BRE-Luc cells. Mouse Twisted Gastrulation (TSG) was from R&D Systems (2013 cat#756-TG). Human CHRDL-1 was from R&D Systems (2013 cat#1808-NR). Mouse CHRDL-1 was produced and purified essentially as described herein. CHRDL-1 assays were run in the presence of BMP4 and TSG.

Cell culture was performed at 37° C. and 5% CO₂. MC3T3-E1-BRE-Luc cells were passaged and maintained in Alpha-MEM (cat #12571-048, Gibco-Invitrogen), 10% fetal bovine serum (cat #10099-141, Invitrogen), 500 ug/ml Geneticin (cat #10131-027, Invitrogen), 1× penicillin-streptomycin (Pen Strep, cat #15140, Gibco-Invitrogen), and 1× Glutamax (cat #35050-061, Gibco-Invitrogen). For the BMP signaling assay, MC3T3-E1-BRE-Luc cells were plated at 20,000 per well in 96-well microtiter plates (BIOCOAT Collagen I coated white/opaque plate—Becton Dickinson cat #354519). Human BMP4, TSG, huCHRDL-1, mCHRDL-1 and antibodies against CHRDL-1 were included in the wells as appropriate (See FIG. 6). After overnight cell culture (e.g. 16-24 hours) luciferase levels were determined as relative light unit (RLU) values in each well using Bright-Glo (cat #PAE2620, Promega). For the experiments set forth in FIG. 6, BMP4 was used at 10 ng/ml, TSG was used at 0.4 ug/ml, huCHRDL-1 was used at 2.5 ug/ml, mCHRDL-1 was used at 2.5 ug/ml and the 1E3.1 and 2E1.1 antibodies were used at 20 ug/ml. The antibodies used for the experiments shown in FIG. 6 have molecular weights of about 145 Kd and have 2 CHRDL-1 binding sites per antibody molecule.

In this assay system, BMP4 is used to stimulate BMP signaling. For the results shown in FIG. 6 each treatment group consisted of 6 wells (i.e., N=6). RLUs for each treatment group are shown as means±Standard Error of the Mean (SEM). For statistical analysis a One-Way ANOVA followed by Tukey's Multiple Comparison Test was used to determine differences between treatment groups. Means for each treatment were considered significantly different when the P value was less than 0.05 (P<0.05). In the mouse CHRDL-1 exemplary experiment shown in the FIG. 6 left side panel, the RLU mean was about 564 in the absence of mCHRDL-1. However, inclusion of mCHRDL-1 resulted in a statistically significant reduction in the RLU mean to a value of about 254 (about a 55% reduction) demonstrating that mCHRDL-1 inhibits the BMP signaling. Inclusion of CHRDL-1 antibody 1E3.1 or 2E1.1 along with the mCHRDL-1 resulted in a statistically significant increase in both RLU means (to about 426 and 420 respectively) as compared to the “mouse chordin-like 1 but no antibody” treatment group, because the inhibitory activity of mCHRDL-1 was neutralized by either antibody. The results from this experiment indicate that antibodies 1E3.1 and 2E1.1 are mCHRDL1 neutralizing monoclonal antibodies (Mabs).

In the human CHRDL-1 exemplary experiment shown in the FIG. 6 right side panel, the RLU mean was about 564 in the absence of huCHRDL-1. However, inclusion of huCHRDL-1 resulted in a statistically significant reduction in the RLU mean to a value of about 62 (about an 89% reduction) demonstrating that hCHRDL-1 inhibits BMP signaling. Inclusion of CHRDL-1 antibody 1E3.1 or 2E1.1 along with the huCHRDL-1 resulted in a statistically significant increase in both RLU means (to about 251 and 236 respectively) as compared to the “human chordin-like 1 but no antibody” treatment group, because the inhibitory activity of huCHRDL1 was neutralized by either antibody. The results from this experiment demonstrate that antibodies 1E3.1 and 2E1.1 are huCHRDL-1 neutralizing monoclonal antibodies (Mabs). Another antibody that is able to neutralize huCHRDL-1 activity in this assay is the fully human antibody 16E6.1 as described herein.

### Example 4

### ELISA-Based Cross-Blocking Assay

The terms “cross-block”, “cross-blocked”, and “cross-blocking” are used interchangeably herein to mean the ability of an antibody or other binding agent to interfere with the binding of other antibodies or binding agents to CHRDL-1.

The extent to which an antibody or other binding agent is able to interfere with the binding of another to CHRDL-1, and therefore whether it can be said to cross-block according to the invention, can be determined using competition binding assays. One particularly suitable quantitative cross-blocking assay uses an ELISA-based approach to measure competition between antibodies or other binding agents in terms of their binding to CHRDL-1.

The following generally describes an ELISA assay used for determining whether a CHRDL-1 antibody or other CHRDL-1 binding agent cross-blocks or is capable of cross-blocking according to the invention. For convenience, reference is made to two antibodies (Ab-X and Ab-Y), but it will be appreciated that the assay can be used with any of the CHRDL-1 binding agents described herein.

Generally, a CHRDL-1 antibody is coated onto the wells of an ELISA plate. An excess amount of a second, potentially cross-blocking, CHRDL-1 antibody is pre-incubated in solution with a limited amount of recombinant CHRDL-1 in a separate ELISA plate. This pre-incubated mixture is then added onto the “coated” CHRDL-1 antibody plate. After a suitable incubation time this plate is washed to remove CHRDL-1 that has not been bound by the coated antibody and to also remove the second, solution phase antibody as well as any complexes formed between the second, solution phase antibody and CHRDL-1. The amount of bound recombinant CHRDL-1 is then measured using an appropriate CHRDL-1 detection reagent. An antibody in solution that is able to cross-block the coated antibody will be able to cause a decrease (as defined further below) in the number of CHRDL-1 molecules that the coated antibody can bind relative to the number of CHRDL-1 molecules that the coated antibody can bind in the absence of the second, solution phase, antibody.

This assay is described in more detail further below for Ab-X and Ab-Y. In the instance where Ab-X is chosen to be the immobilized antibody, it is coated onto the wells of the ELISA plate, after which the plates are blocked with a suitable blocking solution to minimize non-specific binding of reagents that are subsequently added.

An excess amount of Ab-Y is then pre-incubated in solution with a limited amount of recombinant CHRDL-1 in a separate ELISA plate such that the moles of Ab-Y CHRDL-1 binding sites per well are at least 10 fold higher than the moles of Ab-X CHRDL-1 binding sites that were used, per well, during the coating of the ELISA plate. Also, the moles of CHRDL-1 pre-incubated with Ab-Y are at least 10-fold lower than the moles of Ab-X CHRDL-1 binding sites that were used for coating each well.

Following a suitable incubation period the ELISA plate is washed and a CHRDL-1 detection reagent is added to measure the amount of recombinant CHRDL-1 specifically bound by the coated CHRDL-1 antibody (in this case Ab-X). The background signal for the assay would be, for example, the signal obtained in wells with the coated antibody (in this case Ab-X), second solution phase antibody (in this case Ab-Y), CHRDL-1 buffer only (i.e. no CHRDL-1) and CHRDL-1 detection reagents. The positive control signal for the assay would be the signal obtained in wells with the coated antibody (in this case Ab-X), second solution phase antibody buffer only (i.e. no second solution phase antibody), CHRDL-1 and CHRDL-1 detection reagents. The ELISA assay needs to be run in such a manner so as to have the positive control signal be at least 5 times the background signal. The ELISA assay needs to be run such that there are at least N=3 wells for each of the following: positive control, cross-blocking and background signal.

To avoid any artifacts (e.g. significantly different affinities between Ab-X and Ab-Y for CHRDL-1) resulting from the choice of which antibody to use as the coating antibody and which to use as the second (competitor) antibody, the cross-blocking assay needs to be run in two formats:
1) Format 1 is where Ab-X is the antibody that is coated onto the ELISA plate and Ab-Y is the competitor antibody that is in solution, and
2) Format 2 is where Ab-Y is the antibody that is coated onto the ELISA plate and Ab-X is the competitor antibody that is in solution.

Ab-X and Ab-Y are defined as cross-blocking if, either in format 1 or in format 2, the solution phase CHRDL-1 antibody is able to cause, after subtraction of the mean background signal value from the experimental values, a statistically significant reduction (P<0.05) of at least 70% or more, specifically of at least 80% or more, of the CHRDL-1 detection signal (i.e. the amount of CHRDL-1 bound by the coated CHRDL-1 antibody in the presence of the solution phase competitor CHRDL-1 antibody) as compared to the positive control CHRDL-1 detection signal (i.e. the amount of CHRDL-1 bound by the coated CHRDL-1 antibody in the absence of the solution phase competitor CHRDL-1 antibody).

An exemplary experiment for testing cross-blocking between antibodies 1E3.1 and 2E1.1; antibodies 1E3.1 and 2G2.2; antibodies 2E1.1 and 2G2.2 was performed as follows.

Antibodies 1E3.1, 2E1.1, and 2G2.2 were added (20 ul per well at 1 ug/ml in PBS) into a 96-well half-area plate (Costar, cat #3694) and placed at 4° C. overnight. This “coated” plate was washed three times with 100 ul per well of washing solution (PBS containing 0.2% Tween 20). 100 ul per well of SuperBlock-T20 blocking solution (Thermo Scientific, Cat. #37536) was added and incubated for one hour at room temperature (RT). The plate was then washed once with 100 ul per well of wash solution (PBS containing 0.2% Tween 20). Into the “coated” plate, 40 ul per well of the pre-incubated “solution CHRDL-1 antibody and recombinant huCHRDL-1” was added and incubated for 1 hour at RT.

The 40 ul came out of a 60 ul pre-incubation mix (pre-incubation was in a 96-well half-area plate for 2 hours at RT) which consisted of 30 ul of 10 ug/ml of either 1E3.1, 2E1.1 or 2G2.2 (antibodies should be diluted at least 10-fold in Superblock-T20 blocking solution from stock solutions) mixed with 30 ul of 25 ng/ml of His-tagged huCHRDL-1 (R&D Systems, 2013 cat#1808-NR), which had been diluted at least 10-fold in Superblock-T20 blocking solution from a stock solution.

After the pre-incubated “solution CHRDL-1 antibody and recombinant huCHRDL-1” had been added to the “coated” plate for the 1 hour RT incubation, the plate was washed three times with wash solution (PBS containing 0.2% Tween 20). 20 ul of biotin labeled mouse anti-His antibody (THE® His Tag Antibody, GenScript, Cat#A00613) diluted 1:2500 in Superblock-T20 was added per well and incubated at RT for 1 hour. The plate was then washed three times with wash solution (PBS containing 0.2% Tween 20). 20 ul of Streptavidin-HRP (BD Pharmingen, cat#554066) diluted 1:2500 in Superblock-T20 was added per well and incubated at RT for 1 hour. The plate was then washed four times with wash solution (PBS containing 0.2% Tween 20). 20 ul of SuperSignal ELISA Femto (Thermo Scientific, cat#37074) working solution was added per well, mixed for about 1 minute and read in a luminometer.

The well coatings of 1E3.1, 2E1.1 and 2G2.2, and the “solution” phase additions of 1E3.1, 2E1.1 and 2G2.2 pre-mixed with huCHRDL-1 were done in an appropriate matrix fashion to determine which antibodies could cross-block each other and/or be cross-blocked by each other (i.e. format 1 and format 2). Additionally, the appropriate “background” and “positive” control wells were included on the plate as already described herein. The mean background signal value was subtracted from the experimental values prior to statistical analysis and determining whether any particular combination of antibodies were cross-blocking (i.e. at least 70% decrease in detection signal).

1E3.1 and 2E1.1 were found to be cross-blocking antibodies (85% decrease in detection signal when 1E3.1 was the “coated” antibody and 2E1.1 was the “solution” antibody; 93% decrease in detection signal when 2E1.1 was the “coated” antibody and 1E3.1 was the “solution” antibody). 2G2.2 was distinct from 1E3.1 and 2E1.1 because 2G2.2 was not cross-blocked by either 1E3.1 or 2E1.1, nor was 2G2.2 able to cross-block either of 1E3.1 or 2E1.1.

Antibodies 1H6.2, TC3.2.1, 3B9.1, 3C11.2, 1A11.2, 1G12.1, 3B2.1, 3G4.1 and 3H6.2 were found to be cross-blocked by, and/or to cross-block the 2G2.2 antibody. Antibodies 1H6.2, TC3.2.1, 3B9.1, 3C11.2, 1A11.2, 1G12.1, 3B2.1, 3G4.1 and 3H6.2 were found to not be cross-blocked by, nor able to cross-block the 1E3.1 antibody.

It will be appreciated by those in the art that in addition to detecting a His-tagged CHRDL-1 as described above, other tags and tag binding protein combinations that are known in the art could be used in this ELISA-based cross-blocking assay (e.g., HA tag with anti-HA antibodies; FLAG tag with anti-FLAG antibodies; biotin tag with streptavidin).

### Example 5

### Metabolic Effect of High Fat Diet (HFD) on CHRDL-1 Knockout Mice

Targeted CHRDL-1 knockout mouse embryonic stem cells are available commercially from the KnockOut Mouse Project (e.g., cell lines Chrdl1^{tm1a(KOMP)Wtsi}, Chrdl1^{tm2a(KOMP)Wtsi}, and Chrdl1^{tm2e(KOMP)Wtsi}).

The CHRDL-1 knockout mice used herein were produced as previously described (see e.g., Example 4 of U.S. Pat. No. 6,503,712).

Eight wild-type and eight CHRDL-1 knockout male mice were housed at 28° C. at ˜6 weeks of age and challenged with a high-fat diet (60% kcal from fat, Research Diets D12492) at age ˜8 weeks. Weekly body weights were taken (FIG. 3) and after eight weeks on HFD mice were subjected to glucose tolerance tests (GTTs) as follows. All mice were fasted for four hours from 8:00 AM to 12:00 PM with free access to water. Whole blood was collected by tail nick at time 0 and subsequently 1 mg/kg of D-glucose was introduced by intraperitoneal injection and blood collected at time 15, 30, 60, 90, and 120 minutes. Blood glucose was measured by hand-held glucometer (AlphaTRAK™, Abbott Labs) (See FIG. 3). Mice were euthanized after nine weeks of HFD and blood collected by cardiac puncture into serum separator tubes (BD Microtainer 365956) for clinical chemistry analysis including HDL-C and LDL-C (FIG. 5). Adipose tissues were then excised and immediately fixed in 10% formalin. Following paraffin embedding, Sum sections were cut and immunohistochemistry performed using anti-UCP1 antibodies (Abcam 10983) at 0.65 ug/ml (See FIG. 4).

The results from this experiment demonstrate that the lack of CHRDL-1 activity effectively reduces weight gain and maintains glucose tolerance in animals challenged with a high-fat, diabetogenic diet. Further, the absence of CHRDL-1 promotes the formation and maintenance of brown adipose tissue following a high-fat, diabetogenic diet.

### Example 6

### Metabolic Effect of High Fat Diet (HFD) on Mice Treated Concurrently With CHRDL-1 Monoclonal Antibodies

C57B16 male mice (Taconic) were housed at 22° C., and at ˜6 weeks of age challenged with a high-fat diet (HFD) of 60% kcal from fat (Research Diets D12492). Three days after the start of HFD, groups of mice (N=10) were injected intraperitoneally with either 5 mg/kg (2G2.2) or 10 mg/kg (1E3.1) CHRDL-1 monoclonal antibodies or vehicle once per week for 10 weeks (i.e., 10 injection days). After the 4^{th} injection day and before the 5^{th} injection day the mice were moved from the 22° C. room to a 30° C. room (thermoneutrality). Weekly body weights were taken during their time at 22° C. and 30° C. (FIG. 9) and, after ten weeks on HFD, mice were subjected to glucose tolerance tests (GTTs) as follows. All mice were fasted for five hours from 8:00 AM to 1:00 PM with free access to water. Whole blood was collected by tail nick at time 0 and subsequently 1 mg/kg of D-glucose was introduced by intraperitoneal injection and blood collected at time 15, 30, 60, 90, and 120 minutes. Blood glucose was measured by hand-held glucometer (AlphaTRAK, Abbott Labs) (See FIG. 9). Mice were euthanized after ten weeks of HFD and blood collected by cardiac puncture into serum separator tubes (BD Microtainer 365956) for clinical chemistry analysis including total cholesterol and triglycerides, HDL-C, and LDL-C (FIG. 11). 10 μl of serum was used for serum metabolic hormone analysis (FIG. 10) using a multi-plex array system (Milliplex MAP Mouse Metabolic Immunoassay, Millipore MMHMAG-44K-14). Administration of antibody 2G2.2 resulted in a statistically significant reduction in weight gain on HFD as compared to the vehicle group (FIG. 9). Additionally, there was a statistically significant improvement in glucose tolerance in the 2G2.2 treated mice as compared to the vehicle group (See FIG. 9).

### Example 7

### Purification of Recombinant huCHRDL-1-DEVD-6xHis

Recombinant huCHRDL-1-DEVD-6xHis was purified from mammalian host cells as described below. As with the purification of muCHRDL-1-DEVD-6XHis, the processes were carried out at 4° C. The purification scheme also used metal affinity chromatography followed by cation exchange chromatography, but for huCHRDL-1-DEVD-6xHis a size exclusion chromatography was employed as a third step.

Metal Affinity Chromatography

The mammalian host cell conditioned medium (CM) was clarified by centrifugation and filtration using the method employed for muCHRDL-1-DEVD-6XHis. The media was loaded directly onto a Ni-Sepharose excel column (GE Healthcare) equilibrated in 20 mM sodium phosphate, 0.5 M NaCl, 20 mM imidazole, pH 7.4.

After loading, the Ni-Sepharose column was washed with 20 mM sodium phosphate, 0.5 M NaCl, 20 mM imidazole, pH 7.4 with 3 column volumes or until the absorbance at 280 nm of the flow-through returned to pre-load baseline. The huChrdl-1-DEVD-6xHis was then eluted from the column using a linear gradient from 20 mM to 330 mM imidazole in 20 mM sodium phosphate, 0.5 M NaCl, pH 7.4. The absorbance at 280 nm of the eluate was monitored and fractions containing protein were collected. The fractions were then assayed by Coomassie-stained SDS-PAGE to identify fractions containing a polypeptide that migrated at the expected size of human CHRDL-1 with the C-terminal DEVD-6xHis tag. The appropriate fractions from the column were combined to make the Ni Sepharose pool.

Cation Exchange Chromatography

The Ni-Sepharose pool was buffer exchanged into 20 mM NaOAc, 100 mM NaCl, pH 5.0 by dialysis using 10,000 MWCO membranes (Pierce Slide-A-Lyzer). The dialyzed Ni-Sepharose pool was then loaded onto a CM Sepharose Fast Flow column (GE Healthcare) equilibrated in 20 mM NaOAc, 100 mM NaCl, pH 5.0. After loading, the column was washed with 20 mM NaOAc, 100 mM NaCl, pH 5.0 with 3 column volumes or until the absorbance at 280 nm of the flow-through returned to baseline. The huCHRDL-1-DEVD-6xHis was then eluted from the CM Sepharose column using a linear gradient from 100 mM to 500 mM sodium chloride in 20 mM NaOAc, pH 5.0. The absorbance at 280 nm of the eluate was monitored and the eluted huCHRDL-1-DEVD-6xHis was collected in fractions. The fractions were then assayed by Coomassie-stained SDS-PAGE to identify fractions containing a polypeptide that migrated at the expected size of huChrdl-1-DEVD-6xHis. The appropriate fractions were combined to make the CM Sepharose pool.

Size Exclusion Chromatography

The huCHRDL-1-DEVD-6xHis was further purified by size exclusion chromatography employing Superdex 75 resin (GE Healthcare). The CM Sepharose pool was concentrated to 3.5 mg/ml using VIVASPIN 20 5,000 MWCO PES (Vivaproducts) in a Beckman J6-M1 centrifuge at 2500 rpm. The running buffer was 20 mM NaOAc, 250 mM NaCl, pH 5.0. The load volume was <5% of column volume. Fractions containing absorbance at 280 nm were then assayed by Coomassie-stained SDS-PAGE to identify fractions containing a polypeptide that migrated at the expected size of huChrdl-1-DEVD-6xHis. The appropriate fractions were combined to make the Superdex 75 pool.

Formulation

Following purification, the Superdex 75 pool was formulated in 10 mM NaOAc, 150 mM NaCl, pH 5.0 by dialysis using 10,000 MWCO membranes (Pierce Slide-A-Lyzer). Following formulation, the huCHRDL-1-DEVD-6xHis was filtered through a sterile 0.2 μm cellulose acetate filter and stored in aliquots at −70° C.

### Example 8

### KinExA®-Based Determination of Affinity (K_{D}) of Anti-CHRDL-1 Antibodies to huCHRDL-1

Binding of the various anti-CHRDL-1 antibodies to huCHRDL-1-DEVD-6xHis was performed using KinExA technology. The huCHRDL-1 protein was coated onto Reacti-Gel™ 6X (Pierce Biotechnology, Inc., Rockford, Ill.) agarose beads at a concentration of 50 ug protein/mL of beads in 50 mM Na₂CO₃ pH 9.6 for approximately 18 hours at 4° C. The coated beads were then blocked with 1 mg/mL BSA (Sigma-Aldrich, St. Louis, Mo.) in 1 M Tris-HCl, pH 7.5 for 2 hours at 4° C. Antibodies at fixed concentrations were mixed with varying concentrations of huCHRDL-1 in PBS containing 0.1 mg/mL BSA and 0.005% polysorbate 20 (P-20, BIAcore, Inc., Piscataway, N.J.) for at least 12 hours at room temperature. Antibody concentrations were 10 pM, 30 pM, 100 pM and/or 300 pM as appropriate. Following incubation, the sample mixtures were then passed over the huCHRDL-1-coated beads. Antibody bound to the beads was quantified using fluorescent DyLight 649-conjugated goat anti-murine IgG (H+L) antibody (Jackson ImmunoResearch, West Grove, Pa.) at 1 ug/mL in Super Block (Pierce Biotechnology, Inc., Rockford, Ill.) for the mouse antibodies, or using AlexaFluor™ 647-conjugated goat anti-human IgG (H+L) at 1 ug/mL for the humanized antibody. Because only free antibody is able to bind to the huCHRDL-1-coated beads, the binding signal obtained at a given huCHRDL-1 concentration is proportional to the amount of free antibody in solution at equilibrium. The dissociation equilibrium constant (K_{D}) can be determined from nonlinear regression analysis of the competition curves using a multiple curve one-site homogeneous binding model provided in the KinExA Pro software (Sapidyne Instruments, Inc., Boise, Id.).

The humanized antibody having light chain huz-LC2G2.2_LC (SEQ ID NO: 65) and heavy chain huz-bmHC2G2.2_eflsIgG1 (SEQ ID NO: 72) is referred to herein as huz-LC2_G2.2_LC/huz-bmHC2G2.2_eflsIgG1 or huz-2G2.2-A.

Two binding curves were generated for the mouse anti-CHRDL-1 antibodies 2G2.2 and 3C11.2. Only one curve was generated for the mouse anti-CHRDL-1 antibody 1E3.1, as the binding affinity was much weaker compared to the other murine antibodies, and was at the upper range of accuracy for the KinExA system. Three binding curves were generated for huz-2G2.2-A.

Results of the KinExA assays for the selected antibodies are summarized in the table below.

| | | | 95% confidence |
|---|---|---|---|
| Antibody | Antigen | K_{D} (pM) | interval |
| Murine 2G2.2 | Human CHRDL-1 | 16 | 12 pM-22 pM |
| huz-2G2.2-A | Human CHRDL-1 | 142 | 90 pM-210 pM |
| Murine 3C11.2 | Human CHRDL-1 | 2 | <3.2 pM |
| Murine 1E3.1 | Human CHRDL-1 | 640 | 320 pM-1.2 nM |

### Example 9

### Molecular Effect of CHRDL-1 on Adipose Tissue

CHRDL-1 wild-type (WT, N=6) and knock-out (KO, N=6) male mice were housed at 28° C. at ˜6 weeks of age. Six weeks later white adipose tissue (WAT) and brown adipose tissue (BAT) were collected. Ribonucleic acids (RNA) were isolated and analyzed using the GeneChip Mouse Genome 430 2.0 Array (Affymetrix) as per the manufacturer's protocol.

### Example 10

### Metabolic Effect of CHRDL-1 Monoclonal Antibodies on Obese Mice

C57B16 male mice (Charles River) were housed at 28° C., and at ˜6 weeks of age challenged with a high-fat diet (HFD) of 60% kcal from fat (Research Diets D12492). Six weeks after the start of HFD, groups of mice were injected intraperitoneally with either vehicle (N=10) or 10 mg/kg CHRDL-1 monoclonal antibodies (2G2.2, N=8 or 2E1.1, N=10) once per week for 6 weeks (i.e., 6 injection days). Weekly body weights were taken (FIG. 15) and, after twelve weeks on HFD, mice were subjected to glucose tolerance tests (GTTs) as follows. All mice were fasted for five hours from 8:00 AM to 1:00 PM with free access to water. Whole blood was collected by tail nick at time 0 and subsequently 1 mg/kg of D-glucose was introduced by intraperitoneal injection and blood collected at time 15, 30, 60, 90, and 120 minutes. Blood glucose was measured by hand-held glucometer (AlphaTRAK™, Abbott Labs) (See FIG. 15). Administration of antibody 2G2.2 resulted in a statistically significant reduction in weight gain on HFD as compared to the vehicle group (FIG. 15). Additionally, there was a statistically significant improvement in glucose tolerance in the 2G2.2 treated mice as compared to the vehicle group (FIG. 15).

### Example 11

### Metabolic Effect of Humanized CHRDL-1 Monoclonal Antibodies on Obese Mice

C57B16 male mice (Charles River) were housed at 28° C., and at ˜6 weeks of age challenged with a high-fat diet (HFD) of 60% kcal from fat (Research Diets D12492). Six weeks after the start of HFD, groups of mice were injected intraperitoneally with either vehicle (N=11), 10 mg/kg CHRDL-1 mouse monoclonal antibodies (2G2.2, N=12 or 1H6.2, N=11), or 10 mg/kg CHRDL-1 humanized monoclonal antibody Huz-2G2.2-A (N=12) once per week for 7 weeks (i.e., 7 injection days). Weekly body weights were taken (FIG. 16) and, after thirteen weeks on HFD whole blood was collected. Administration of antibodies 2G2.2 and Huz-2G2.2-A resulted in statistically significant reductions in weight gain on HFD as compared to the vehicle group (FIG. 16).

### Example 12

### Effect of Monoclonal Antibody 2G2.2 on Brown Adipose Tissue in High Fat Diet Fed Mice

C57B16 male mice (Charles River) were housed at 28° C., and at ˜6 weeks of age challenged with a high-fat diet (HFD) of 60% kcal from fat (Research Diets D12492). Six weeks after the start of HFD, groups of mice were injected intraperitoneally with either vehicle (N=11), or 10 mg/kg CHRDL-1 mouse monoclonal 2G2.2 antibodies once per week for 7 weeks (i.e., 7 injection days). Mice were euthanized after thirteen weeks of HFD and adipose tissues were then excised and immediately fixed in 10% formalin. Following paraffin embedding, Sum sections were cut and stained with hematoxylin and eosin (FIG. 27). The high-fat diet challenge in the vehicle treated mice resulted in the conversion of brown adipose tissue (BAT) to white adipose tissue (WAT). However, the 2G2.2 antibody treatment preserved brown adipose tissue (i.e., prevented conversion of BAT to WAT) in animals challenged with a HFD.

Each reference cited herein is incorporated by reference in its entirety for all that it teaches and for all purposes.

The present disclosure is not to be limited in scope by the specific embodiments described herein, which are intended as illustrations of individual aspects of the disclosure, and functionally equivalent methods and components form aspects of the disclosure. Indeed, various modifications of the disclosure, in addition to those shown and described herein will become apparent to those skilled in the art from the foregoing description and accompanying drawings. Such modifications are intended to fall within the scope of the appended claims.

What is claimed:

1. A method of treating diabetes in a patient comprising administering to said patient an effective amount of an antigen binding protein capable of inhibiting an activity of CHRDL-1.

2. A method of treating a diabetes-related condition or disorder in a patient comprising administering to said patient an effective amount of an antigen binding protein capable of inhibiting an activity of CHRDL-1.

3. The method according to claim 2, wherein said diabetes-related condition or disorder is at least one of diabetic retinopathy or diabetic nephropathy.

4. A method of treating obesity in a patient comprising administering to said patient an effective amount of an antigen binding protein capable of inhibiting an activity of CHRDL-1.

5. A method of modulating blood glucose in a patient comprising administering to said patient an effective amount of an antigen binding protein capable of inhibiting an activity of CHRDL-1.

6. A method of inducing and/or preserving brown fat formation and/or activity in a patient comprising administering to said patient an effective amount of an antigen binding protein capable of inhibiting an activity of CHRDL-1.

7. A method of treating insulin resistance in a patient comprising administering to said patient an effective amount of an antigen binding protein capable of inhibiting an activity of CHRDL-1.

8. A method of treating inflammatory disease in a patient comprising administering to said patient an effective amount of an antigen binding protein capable of inhibiting an activity of CHRDL-1.

9. A method of treating dyslipidemia in a patient comprising administering to said patient an effective amount of an antigen binding protein capable of inhibiting an activity of CHRDL-1.

10. A method of treating a disease or disorder characterized by undesired levels of triglycerides in a patient comprising administering to said patient an effective amount of an antigen binding protein capable of inhibiting an activity of CHRDL-1.

11. The method according to claims 1-10, wherein said antigen binding protein is an antibody.

12. The method according to claims 1-10, wherein said antigen binding protein is a humanized antibody.

13. The method according to claims 1-10, wherein said antigen binding protein is an antibody comprising SEQ ID NO: 8, SEQ ID NO: 9, and SEQ ID NO: 10.

14. The method according to claims 1-10, wherein said antigen binding protein is an antibody comprising SEQ ID NO: 20, SEQ ID NO: 21, and SEQ ID NO: 22.

15. The method according to claims 1-10, wherein said antigen binding protein is an antibody comprising SEQ ID NO: 26, SEQ ID NO: 27 and SEQ ID NO: 28.

16. The method according to claims 1-10, wherein said antigen binding protein is an antibody comprising SEQ ID NO: 37, SEQ ID NO: 38 and SEQ ID NO: 39.

17. The method according to claims 1-10, wherein said antigen binding protein is an antibody comprising SEQ ID NO: 40, SEQ ID NO: 41 and SEQ ID NO: 42.

18. An isolated antigen binding protein comprising SEQ ID NO: 8, SEQ ID NO: 9, and SEQ ID NO: 10.

19. An isolated antigen binding protein comprising SEQ ID NO: 20, SEQ ID NO: 21, and SEQ ID NO: 22.

20. An isolated antigen binding protein comprising SEQ ID NO: 26, SEQ ID NO: 27 and SEQ ID NO: 28.

21. An isolated antigen binding protein comprising SEQ ID NO: 37, SEQ ID NO: 38 and SEQ ID NO: 39.

22. An isolated antigen binding protein comprising SEQ ID NO: 40, SEQ ID NO: 41 and SEQ ID NO: 42.

23. An antibody comprising SEQ ID NO: 49.

24. An antibody comprising SEQ ID NO: 50.

25. An antibody comprising SEQ ID NO: 55.

26. An antibody comprising SEQ ID NO: 56.

27. An antibody comprising SEQ ID NO: 57.

28. An antibody comprising SEQ ID NO: 58.

29. An antibody comprising SEQ ID NO: 59.

30. An antibody comprising SEQ ID NO: 60.

31. An antibody comprising SEQ ID NO: 61.

32. An antibody comprising SEQ ID NO: 62.

33. An antibody comprising SEQ ID NO: 65.

34. An antibody comprising SEQ ID NO: 66.

35. An antibody comprising SEQ ID NO: 69.

36. An antibody comprising SEQ ID NO: 70.

37. An antibody comprising SEQ ID NO: 71.

38. An antibody comprising SEQ ID NO: 72.

39. An antibody that is cross-blocked by, or is capable of cross-blocking, an antigen binding protein of claims 18-20.

40. An antibody that is cross-blocked by, or is capable of cross-blocking, an antigen binding protein of claim 21 or 22.

41. An antibody that is cross-blocked by, or is capable of cross-blocking an antibody of any of claims 23-32.

42. An antibody that is cross-blocked by, or is capable of cross-blocking an antibody of any of claims 33-38.

43. A pharmaceutical composition comprising the antibody of any of claims 23-38.

44. The antigen binding protein of claim 1, wherein the antigen binding protein is a human antibody, a humanized antibody, chimeric antibody, a monoclonal antibody, a polyclonal antibody, a recombinant antibody, an antigen-binding antibody fragment, a single chain antibody, a diabody, a triabody, a tetrabody, a Fab fragment, an F(fab′)₂ fragment, a domain antibody, an IgD antibody, an IgE antibody, an IgM antibody, an IgG1 antibody, an IgG2 antibody, an IgG3 antibody, an IgG4 antibody, or an IgG4 antibody having at least one mutation in the hinge region.

45. A nucleic acid comprising any of SEQ ID NOS: 5, 6, 7, 11, 12, 13, 17, 18, 19, 23, 24, 25, 30, 32, 34, 36, 44, 46, 73, 74, 75, 76, 77, and 78.

46. An expression vector comprising at least one nucleic acid of claim 45.

47. An isolated cell comprising the expression vector of claim 46.

48. A method of producing an antigen binding protein comprising incubating the host cell of claim 47 under conditions that allow it to express the antigen binding protein.

49. A method of preventing or treating a condition in a subject in need of such treatment comprising administering a therapeutically effective amount of the composition of claim 48 to the subject, wherein the condition is treatable by lowering one or more of blood glucose, insulin, or serum lipid levels.

50. The method of claim 49, wherein the condition is type 2 diabetes, obesity, dyslipidemia, NASH, cardiovascular disease or metabolic syndrome.

51. An isolated antigen binding protein having at least 85% sequence identity with any of SEQ ID NO: 8, 9, 10, 20, 21, 22, 26, 37, 38, 39, 40, 41, and 42.

52. An isolated antigen binding protein having at least 90% sequence identity with any of SEQ ID NO: 8, 9, 10, 20, 21, 22, 26, 37, 38, 39, 40, 41, and 42.

53. An isolated antigen binding protein having at least 95% sequence identity with any of SEQ ID NO: 8, 9, 10, 20, 21, 22, 26, 37, 38, 39, 40, 41, and 42.

54. An isolated antigen binding protein comprising a variable region light chain having at least 85% sequence identity with any of light chain SEQ ID NOs: 29, 43, 47, 48, 63, and 64.

55. An isolated antigen binding protein comprising a variable region light chain having at least 90% sequence identity with any of light chain SEQ ID NOs: 29, 43, 47, 48, 63, and 64.

56. An isolated antigen binding protein comprising a variable region light chain having at least 95% sequence identity with any of light chain SEQ ID NOs: 29, 43, 47, 48, 63, and 64.

57. An isolated antigen binding protein comprising a variable region heavy chain having at least 85% sequence identity with any of light chain SEQ ID NOs: 31, 35, 45, 51, 52, 53, 54, 67, and 68.

58. An isolated antigen binding protein comprising a variable region heavy chain having at least 90% sequence identity with any of light chain SEQ ID NOs: 31, 35, 45, 51, 52, 53, 54, 67, and 68.

59. An isolated antigen binding protein comprising a variable region heavy chain having at least 95% sequence identity with any of light chain SEQ ID NOs: 31, 35, 45, 51, 52, 53, 54, 67, and 68.

60. An isolated antigen binding protein comprising a variable region light chain having at least 85% sequence identity with any of light chain SEQ ID NOs: 85, 93, 101, 109, 117, 125, 133, 141, and 149.

61. An isolated antigen binding protein comprising a variable region light chain having at least 90% sequence identity with any of light chain SEQ ID NOs: 85, 93, 101, 109, 117, 125, 133, 141, and 149.

62. An isolated antigen binding protein comprising a variable region light chain having at least 95% sequence identity with any of light chain SEQ ID NOs: 85, 93, 101, 109, 117, 125, 133, 141, and 149.

63. An isolated antigen binding protein comprising a variable region heavy chain having at least 85% sequence identity with any of light chain SEQ ID NOs: 86, 94, 102, 110, 118, 126, 134, 142, and 150.

64. An isolated antigen binding protein comprising a variable region heavy chain having at least 90% sequence identity with any of light chain SEQ ID NOs: 86, 94, 102, 110, 118, 126, 134, 142, and 150.

65. An isolated antigen binding protein comprising a variable region heavy chain having at least 95% sequence identity with any of light chain SEQ ID NOs: 86, 94, 102, 110, 118, 126, 134, 142, and 150.

66. An isolated antigen binding protein comprising at least one of:
(a) SEQ ID NO: 79, SEQ ID NO: 80 and SEQ ID NO: 81;
(b) SEQ ID NO: 82, SEQ ID NO: 83 and SEQ ID NO: 84;
(c) SEQ ID NO: 87, SEQ ID NO: 88 and SEQ ID NO: 89;
(d) SEQ ID NO: 90, SEQ ID NO: 91 and SEQ ID NO: 92;
(e) SEQ ID NO: 95, SEQ ID NO: 96 and SEQ ID NO: 97;
(f) SEQ ID NO: 98, SEQ ID NO: 99 and SEQ ID NO: 100;
(g) SEQ ID NO: 103, SEQ ID NO: 104 and SEQ ID NO: 105;
(h) SEQ ID NO: 106, SEQ ID NO: 107 and SEQ ID NO: 108;
(i) SEQ ID NO: 111, SEQ ID NO: 112, and SEQ ID NO: 113;
(j) SEQ ID NO: 114, SEQ ID NO: 115, and SEQ ID NO: 116;
(k) SEQ ID NO: 119, SEQ ID NO: 120 and SEQ ID NO: 121;
(l) SEQ ID NO: 122, SEQ ID NO: 123, and SEQ ID NO: 124;
(m) SEQ ID NO: 127, SEQ ID NO: 128 and SEQ ID NO: 129;
(n) SEQ ID NO: 130, SEQ ID NO: 131 and SEQ ID NO: 132;
(o) SEQ ID NO: 135, SEQ ID NO: 136 and SEQ ID NO: 137;
(p) SEQ ID NO: 138, SEQ ID NO: 139 and SEQ ID NO: 140;
(q) SEQ ID NO: 143, SEQ ID NO: 144 and SEQ ID NO: 145; and
(r) SEQ ID NO: 146, SEQ ID NO: 147 and SEQ ID NO: 148.

67. A method of converting white adipose tissue to brown adipose tissue comprising administering to a patient an effective amount of a selective binding agent to CHRDL-1.

68. A method of promoting brown adipose tissue production comprising administering to a patient an effective amount of a selective binding agent to CHRDL-1.

69. A method of preventing conversion of brown adipose tissue to white adipose tissue comprising administering to a patient an effective amount of a selective binding agent to CHRDL-1.
